(19) United States
(12) Patent Application Publication
(10) Pub. No.: US 2023/0410985 A1
(43) Pub. Date: Dec. 21, 2023

(54) SYSTEMS AND METHODS FOR ASSESSING DISEASE BURDEN AND PROGRESSION

(71) Applicants: EXINI Diagnostics AB, Lund (SE); Progenics Pharmaceuticals, Inc., N. Billerica, MA (US)

(72) Inventors: Johan Martin Brynolfsson, Helsingborg (SE); Hannicka Maria Eleonora Sahlstedt, Malmö (SE); Jens Filip Andreas Richter, Staffanstorp (SE); Karl Vilhelm Sjöstrand, Atlantic Highlands, NJ (US); Aseem Undvall Anand, Queens, NY (US)

(21) Appl. No.: 18/207,246

(22) Filed: Jun. 8, 2023

Related U.S. Application Data
(60) Provisional application No. 63/461,486, filed on Apr. 24, 2023.
(60) Provisional application No. 63/458,031, filed on Apr. 7, 2023.
(60) Provisional application No. 63/350,211, filed on Jun. 8, 2022.

(51) Int. Cl. G16H 30/40; G16H 50/30; G06T 7/11

(52) U.S. Cl. CPC G16H 30/40; G16H 50/30; G06T 7/11; G06T 2207/10028; G06T 2207/30096

(57) ABSTRACT

Presented herein are systems and methods that provide semi-automated and/or automated analysis of medical image data to determine and/or convey values of metrics that provide a picture of a patient's risk and/or disease. Technologies described herein include systems and methods for analyzing medical image data to evaluate quantitative metrics that provide snapshots of patient disease burden at particular times and/or for analyzing images taken over time to produce a longitudinal dataset that provides a picture of how a patient's risk and/or disease evolves over time during surveillance and/or in response to treatment. Metrics computed via image analysis tools described herein may themselves be used as quantitative measures of disease burden and/or may be linked to clinical endpoints that seek to measure and/or stratify patient outcomes. Accordingly, image analysis technologies of the present disclosure may be used to inform clinical decision making, evaluate of treatment efficacy, and predict patient response(s).

### CROSS-REFERENCE TO RELATED APPLICATIONS

This application claims priority to and benefit of U.S. Provisional Application No. 63/350,211, filed Jun. 8, 2022, U.S. Provisional Application No. 63/458,031, filed on Apr. 7, 2023, and U.S. Provisional Application No. 63/461,486, filed on Apr. 24, 2023, the contents of each of which is hereby incorporated by reference in its entirety.

### FIELD

This invention relates generally to systems and methods for creation, analysis, and/or presentation of medical image data. More particularly, in certain embodiments, the invention relates to systems and methods for automated analysis of medical images to identify and/or characterize cancerous lesions and/or prognosis or risk for a subject.

### BACKGROUND

Nuclear medicine imaging involves the use of radiolabeled compounds, referred to as radiopharmaceuticals. Radiopharmaceuticals are administered to patients and accumulate in various regions in the body in manner that depends on, and is therefore indicative of, biophysical and/or biochemical properties of tissue therein, such as those influenced by presence and/or state of disease, such as cancer. For example, certain radiopharmaceuticals, following administration to a patient, accumulate in regions of abnormal osteogenesis associated with malignant bone lesions, which are indicative of metastases. Other radiopharmaceuticals may bind to specific receptors, enzymes, and proteins in the body that are altered during evolution of disease. After administration to a patient, these molecules circulate in the blood until they find their intended target. The bound radiopharmaceutical remains at the site of disease, while the rest of the agent clears from the body.

Nuclear medicine imaging techniques capture images by detecting radiation emitted from the radioactive portion of the radiopharmaceutical. The accumulated radiopharmaceutical serves as a beacon so that an image may be obtained depicting the disease location and concentration using commonly available nuclear medicine modalities. Examples of nuclear medicine imaging modalities include bone scan imaging (also referred to as scintigraphy), single-photon emission computerized tomography (SPECT), and positron emission tomography (PET). Bone scan, SPECT, and PET imaging systems are found in most hospitals throughout the world. Choice of a particular imaging modality depends on and/or dictates the particular radiopharmaceutical used. For example, technetium 99m (^{99m}Tc) labeled compounds are compatible with bone scan imaging and SPECT imaging, while PET imaging often uses fluorinated compounds labeled with 18F. The compound ^{99m}Tc methylenediphosphonate (^{99m}Tc MDP) is a popular radiopharmaceutical used for bone scan imaging in order to detect metastatic cancer. Radiolabeled prostate-specific membrane antigen (PSMA) targeting compounds such as ^{99m}Tc labeled 1404 and PyL™ (also referred to as [18F]DCFPyL) can be used with SPECT and PET imaging, respectively, and offer the potential for highly specific prostate cancer detection.

Accordingly, nuclear medicine imaging is a valuable technique for providing physicians with information that can be used to determine the presence and the extent of disease in a patient. The physician can use this information to provide a recommended course of treatment to the patient and to track the progression of disease.

For example, an oncologist may use nuclear medicine images from a study of a patient as input in her assessment of whether the patient has a particular disease, e.g., prostate cancer, what stage of the disease is evident, what the recommended course of treatment (if any) would be, whether surgical intervention is indicated, and likely prognosis. The oncologist may use a radiologist report in this assessment. A radiologist report is a technical evaluation of the nuclear medicine images prepared by a radiologist for a physician who requested the imaging study and includes, for example, the type of study performed, the clinical history, a comparison between images, the technique used to perform the study, the radiologist's observations and findings, as well as overall impressions and recommendations the radiologist may have based on the imaging study results. A signed radiologist report is sent to the physician ordering the study for the physician's review, followed by a discussion between the physician and patient about the results and recommendations for treatment.

Thus, the process involves having a radiologist perform an imaging study on the patient, analyzing the images obtained, creating a radiologist report, forwarding the report to the requesting physician, having the physician formulate an assessment and treatment recommendation, and having the physician communicate the results, recommendations, and risks to the patient. The process may also involve repeating the imaging study due to inconclusive results, or ordering further tests based on initial results. If an imaging study shows that the patient has a particular disease or condition (e.g., cancer), the physician discusses various treatment options, including surgery, as well as risks of doing nothing or adopting a watchful waiting or active surveillance approach, rather than having surgery.

Accordingly, the process of reviewing and analyzing multiple patient images, over time, plays a critical role in the diagnosis and treatment of cancer. There is a significant need for improved tools that facilitate and improve accuracy of image review and analysis for cancer diagnosis and treatment. Improving the toolkit utilized by physicians, radiologists, and other healthcare professionals in this manner provides for significant improvements in standard of care and patient experience.

### SUMMARY

Presented herein are systems and methods that provide semi-automated and/or automated analysis of medical image data to determine and/or convey values of metrics that provide a picture of a patient's risk and/or disease. Technologies described herein include systems and methods for analyzing medical image data to evaluate quantitative metrics that provide snapshots of patient disease burden at particular times and/or for analyzing images taken over time to produce a longitudinal dataset that provides a picture of how a patient's risk and/or disease evolves over time during surveillance and/or in response to treatment. Metrics computed via image analysis tools described herein may themselves be used as quantitative measures of disease burden and/or may be linked to clinical endpoints that seek to measure and/or stratify patient outcomes. Accordingly, image analysis technologies of the present disclosure may be used to inform clinical decision making, evaluate of treatment efficacy, and predict patient response(s).

In certain embodiments, values of patient indices that quantify disease burden are computed by analyzing a 3D nuclear medicine image of a subject in order to identify and quantify sub-regions, referred to as hotspots, that indicate presence of underlying cancerous lesions. A variety of quantitative metrics can be computed for individual hotspots to reflect a severity and/or size of the underlying lesions that they represent. These individual hotspot quantification metrics can then be aggregated to compute values of various patient indices that provide a measure of disease burden and/or risk for a subject overall, and/or within particular tissue regions or lesion sub-classes.

In one aspect, the invention is directed to a method for automatically processing 3D images of a subject to determine values of one or more patient index(indices) that measure (e.g., overall) disease burden and/or risk for a subject, the method comprising: (a) receiving, by a processor of a computing device, a 3D functional image of the subject obtained using a functional imaging modality; (b) segmenting, by the processor, a plurality of 3D hotspot volumes within the 3D functional image, each 3D hotspot volume corresponding to a local region of elevated intensity with respect to its surrounding and representing a potential cancerous lesion within the subject, thereby obtaining a set of 3D hotspot volumes; (c) computing, by the processor, for each particular one of one or more individual hotspot quantification metrics, a value of the particular individual hotspot quantification metric for each individual 3D hotspot volume of the set; and (d) determining, by the processor, the values of the one or more patient index(indices), wherein each of at least a portion of the patient indices is associated with one or more specific individual hotspot quantification metrics and is a function of at least a portion (e.g., substantially all; e.g., a particular subset) of the values of the one or more specific individual hotspot quantification metric(s) computed for the set of 3D hotspot volumes.

In certain embodiments, at least one particular patient index of the one or more patient index values is associated with a single specific individual hotspot quantification metric and is computed as a function (e.g., a mean, a median, a mode, a sum, etc.) of substantially all (e.g., all; e.g., excluding only statistical outliers) values of the specific individual hotspot quantification metric computed for the set of 3D hotspot volumes.

In certain embodiments, the single specific individual hotspot quantification metric is an individual hotspot intensity metric that quantifies intensity within a 3D hotspot volume (e.g., computed, for an individual 3D hotspot volume, as a function of intensities of voxels of the 3D hotspot volume).

In certain embodiments, the individual hotspot intensity metric is a mean hotspot intensity (e.g., computed, for an individual 3D hotspot volume, as a mean of intensities of voxels within the 3D hotspot volume).

In certain embodiments, the particular patient index is computed as a sum of substantially all the values of the individual hotspot intensity metric computed for the set of 3D hotspot volumes.

In certain embodiments, the single specific individual hotspot quantification metric is a lesion volume (e.g., computed for a particular 3D hotspot volume as a sum of volumes of each individual voxel within the particular 3D hotspot volume).

In certain embodiments, (values of) the particular patient index is computed as a sum of substantially all the lesion volume values computed for the set of 3D hotspot volumes (e.g., such that the particular patient index value provides a measure of total lesion volume within the subject).

In certain embodiments, a particular one of the one or more overall patient index(indices) is associated with two or more specific individual hotspot quantification metrics and is computed as a function (e.g., a weighted sum, a weighted mean, etc.) of substantially all values of the two or more specific individual hotspot quantification metric computed for the set of 3D hotspot volumes.

In certain embodiments, the two or more specific individual hotspot quantification metrics comprise (i) an individual hotspot intensity metric and (ii) a lesion volume.

In certain embodiments, the individual hotspot intensity metric is an individual lesion index that maps a value of hotspot intensity to a value on a standardized scale.

In certain embodiments, (values of) the particular patient index is computed as a sum of intensity-weighted lesion (e.g., hotspot) volumes by: for each individual 3D hotspot volume of substantially all the 3D hotspot volumes, weighting a value of the lesion volume by a value of the individual hotspot intensity metric (e.g., computing the product of the lesion volume value and the value of the individual hotspot intensity metric), thereby computing a plurality of intensity-weighted lesion volumes; and computing, as the value of the particular patient index, a sum of substantially all the intensity-weighted lesion volumes.

In certain embodiments, the one or more individual hotspot quantification metrics comprise one or more individual hotspot intensity measures that quantify intensity within a 3D hotspot volume (e.g., computed, for an individual 3D hotspot volume, as a function of intensities of voxels of the 3D hotspot volume).

In certain embodiments, the one or more individual hotspot quantification metric comprise one or more members selected from the group consisting of: a mean hotspot intensity (e.g., computed, for a particular 3D hotspot volume, as a mean of intensities of voxels within the particular 3D hotspot volume); a maximum hotspot intensity (e.g., computed, for a particular 3D hotspot volume, as a maximum of intensities of voxels within the particular 3D hotspot volume); and a median hotspot intensity (e.g., computed, for a particular 3D hotspot volume, as a median of intensities of voxels within the 3D hotspot volume).

In certain embodiments, the one or more individual hotspot intensity metric(s) comprise a peak intensity of a 3D hotspot volume [e.g., wherein, for a particular 3D hotspot volume, a value of the peak intensity is computed by: (i) identifying a maximum intensity voxel within the particular 3D hotspot volume; (ii) identifying voxels within a sub-region about (e.g., comprising voxels within a particular threshold distance of) the maximum intensity voxel and within the particular 3D hotspot; and (iii) computing, as the corresponding peak intensity, a mean of intensities of the voxels within the sub-region].

In certain embodiments, the one or more individual hotspot intensity metrics comprise an individual lesion index that maps a value of hotspot intensity to a value on a standardized scale.

In certain embodiments, the method comprises: identifying, by the processor, within the 3D functional image, one or more 3D reference volume(s), each corresponding to a particular reference tissue region; determining, by the processor, one or more reference intensity values, each associated with a particular 3D reference volume of the one or more 3D reference volume(s) and corresponding to a measure of intensity within the particular 3D reference volume; and at step (c), for each 3D hotspot volume within the set, determining, by the processor, a corresponding value of a particular individual hotspot intensity metric (e.g., a mean hotspot intensity, a median hotspot intensity, a maximum hotspot intensity, etc.); and determining, by the processor, a corresponding value of the individual lesion index based on the corresponding value of the particular individual hotspot intensity metric and the one or more reference intensity values.

In certain embodiments, the method comprises: mapping each of the one or more reference intensity values to a corresponding reference index value on a scale; and for each 3D hotspot volume, determining the corresponding value of the individual lesion index using the reference intensity values and corresponding reference index values to interpolate a corresponding individual lesion index value on the scale based on the corresponding value of the particular individual hotspot intensity metric.

In certain embodiments, the reference tissue regions comprise one or more members selected from the group consisting of: a liver, an aorta, and a parotid gland.

In certain embodiments, a first reference intensity value (i) is a blood reference intensity value associated with a reference volume corresponding to an aorta portion, and (ii) maps to a first reference index value; a second reference intensity value (i) is a liver reference intensity value associated with a reference volume corresponding to a liver, and (ii) maps to a second reference index value; and the second reference intensity value is greater than the first reference intensity value and the second reference index value is greater than the first reference index value.

In certain embodiments, the reference intensity values comprises a maximum reference intensity value that maps to a maximum reference index value, and wherein 3D hotspot volumes for which corresponding values of the particular individual hotspot intensity metric are greater than the maximum reference intensity value are assigned individual lesion index values equal to the maximum reference index value.

In certain embodiments, the method comprises: identifying, within the set of 3D hotspot volumes, one or more subsets, each associated with a particular tissue region and/or lesion classification; and computing, for each particular subset of the one or more subsets, a corresponding value of one or more particular patient index(indices) using values of the individual hotspot quantification metrics computed for 3D hotspot volumes within the particular subset.

In certain embodiments, the one or more subsets is associated with a particular one of one or more tissue region(s) and the method comprises identifying, for each particular tissue region, a subset of the 3D hotspot volumes located within a volume of interest corresponding to the particular tissue region.

In certain embodiments, the one or more tissue region(s) comprise one or more members selected from the group consisting of: a skeletal region comprising one or more bones of the subject, a lymph region, and a prostate region.

In certain embodiments, each of the one or more subsets is associated with a particular one of one or more lesion sub-types [e.g., according to a lesion classification scheme (e.g., a miTNM classification)] and the method comprises determining, for each 3D hotspot volume, a corresponding lesion sub-type and assigning the 3D hotspot volumes to the one or more subsets according to their corresponding lesion sub-types.

In certain embodiments, the method comprises using at least a portion of the values of the one or more patient index(indices) as inputs to a prognostic model (e.g., a statistical model, such as a regression; e.g., a classification model, whereby a patient is assigned to a particular class based on a comparison of the one or more patient index values with one or more thresholds; e.g., a machine learning model, where the values of the one or more patient indices are received as input) that generates, as output, an expectation value and/or range (e.g., a class) indicative of a likely value of a particular patient outcome (e.g., a time, e.g., in number of months, representing an expected survival, time to progression, time to radiographic progression, etc.).

In certain embodiments, the method comprises using at least a portion of the values of the one or more patient index(indices) as inputs to a predictive model (e.g., a statistical model, such as a regression; e.g., a classification model, whereby a patient is assigned to a particular class based on a comparison of the one or more patient index values with one or more thresholds; e.g., a machine learning model, where the values of the one or more patient indices are received as input) that generates, as output, an eligibility score for each of one or more treatment options (e.g., Abiraterone, Enzalutamide, Apalutamide, Darolutamide, Sipuleucel-T, Ra223, Docetaxel, Carbazitaxel, Pembrolizumab, Olaparib, Rucaparib, ¹⁷⁷Lu-PSMA-617, etc.) and/or classes of therapeutics [e.g., androgen biosynthesis inhibitors (e.g., Abiraterone), androgen receptor inhibitors (e.g., Enzalutamide, Apalutamide, Darolutamide), a cellular immunotherapy (e.g., Sipuleucel-T), internal radiotherapy treatment (Ra223), antineoplastics (e.g., Docetaxel, Carbazitaxel), immune checkpoint inhibitor (Pembrolizumab), PARP inhibitors (e.g., Olaparib, Rucaparib), PSMA binding agent], wherein the eligibility score for a particular treatment option and/or therapeutic class indicates a prediction of whether the patient will benefit from the particular treatment and/or therapeutic class.

In certain embodiments, the method comprises generating (e.g., automatically) a report [e.g., an electronic document, e.g., within a graphical user interface (e.g., for validation/sign-off by a user)] comprising at least a portion of the values of the one or more patient index(indices).

In certain embodiments, the method comprises using one or more machine learning modules [e.g., one or more neural networks (e.g., one or more convolutional neural networks)] to perform one or more functions selected from the group consisting of: detecting a plurality of hotspots, wherein each of at least a portion of the plurality of 3D hotspot volumes corresponds to a particular detected hotspot and is produced by segmenting the particular detected hotspot; segmenting at least a portion of the plurality of 3D hotspot volumes; and classifying at least a portion of the 3D hotspot volumes (e.g., determining a likelihood that each 3D hotspot volume represents an underlying cancerous lesion).

In certain embodiments, the 3D functional image comprises a PET or SPECT image obtained following administration of an agent to the subject. In certain embodiments, the agent comprises a PSMA binding agent. In certain embodiments, the agent comprises ¹⁸F. In certain embodiments, the agent comprises [18F]DCFPyL. In certain embodiments, the agent comprises PSMA-11. In certain embodiments, the agent comprises one or more members selected from the group consisting of ^{99m}Tc, ⁶⁸Ga, ¹⁷⁷Lu, ²²⁵Ac, ¹¹¹In, ¹²³I, ¹²⁴I, and ¹³¹I.

In another aspect, the invention is directed to a method for automated analysis of a time series of medical images [e.g., three-dimensional images, e.g., nuclear medicine images (e.g., bone scan (scintigraphy), PET, and/or SPECT), e.g., anatomical images (e.g., CT, X-ray, MRI), e.g., combined nuclear medicine and anatomical images (e.g., overlaid)] of a subject, the method comprising: (a) receiving and/or accessing, by a processor of a computing device, the time series of medical images of the subject; and (b) identifying, by the processor, a plurality of hotspots within each of the medical images and determining, by the processor, one, two, or all three of (i), (ii), and (iii) as follows: (i) a change in the number of identified lesions (ii) a change in an overall volume of identified lesions (e.g., a change in the sum of the volumes of each identified lesion), and (iii) a change in PSMA (e.g., lesion index) weighted total volume (e.g., a sum of the products of lesion index and lesion volume for all lesions in a region of interest) [e.g., wherein the change identified in step (b) is used to identify (1) a disease status [e.g., progression, regression, or no change], (2) make a treatment management decision [e.g. active surveillance, prostatectomy, anti-androgen therapy, prednisone, radiation, radio-therapy, radio-PSMA therapy, or chemotherapy], or (3) treatment efficacy (e.g. wherein the subject has begun treatment or has continued treatment with a medicament or other therapy following an initial set of images in the time series of medical images)] [e.g., wherein step (b) comprises using a machine learning module/model].

In another aspect, the invention is directed to a method for analyzing a plurality of medical images of a subject (e.g., to evaluate disease state and/or progression within the subject), the method comprising: (a) receiving and/or accessing, by a processor of a computing device, the plurality of medical images of the subject and obtaining, by the processor, a plurality of 3D hotspot maps, each corresponding to a particular medical image (of the plurality) and identifying one or more hotspots (e.g., representing potential underlying physical lesions within the subject) within the particular medical image; (b) for each particular one (medical image) of the plurality of medical images, determining, by the processor, using a machine learning module [e.g., a deep learning network (e.g., a Convolutional Neural Network (CNN))], a corresponding 3D anatomical segmentation map that identifies a set of organ regions [e.g., representing soft tissue and/or bone structures within the subject (e.g., one or more of a cervical spine; thoracic spine; lumbar spine; left and right hip bones, sacrum and coccyx; left side ribs and left scapula; right side ribs and right scapula; left femur; right femur; skull, brain and mandible)] within the particular medical image, thereby generating a plurality of 3D anatomical segmentation maps; (c) determining, by the processor, using (i) the plurality of 3D hotspot maps and (ii) the plurality of 3D anatomical segmentation maps, an identification of one or more lesion correspondences, each (lesion correspondence) identifying two or more corresponding hotspots within different medical images and determined (e.g., by the processor) to represent a same underlying physical lesion within the subject; and (d) determining, by the processor, based on the plurality of 3D hotspot maps and the identification of the one or more lesion correspondences, values of one or more metrics {e.g., one or more hotspot quantification metrics and/or changes therein [e.g., that quantify a change in properties, such as volume, radiopharmaceutical uptake, shape, etc. of individual hotspots and/or the underlying physical lesions that they represent (e.g., over time/between multiple medical images)]; e.g., patient indices (e.g., that that measure overall disease burden and/or state and/or risk for a subject) and/or changes thereof; e.g., values classifying a patient (e.g., as belonging to and/or having a particular disease state, progression, etc. category) e.g., prognostic metrics [e.g., indicative of and/or which quantify a likelihood of one or more clinical outcomes (e.g., a disease state, progression, likely survival, treatment efficacy, and the like) (e.g., overall survival); e.g., predictive metrics (e.g., indicative of a predicted response to therapy and/or other clinical outcome)}.

In certain embodiments, the plurality of medical images comprise one or more anatomical images (e.g., CT, X-Ray, MRI, Ultrasound, etc.).

In certain embodiments, the plurality of medical images comprise one or more nuclear medicine images [e.g., bone scan (scintigraphy) (e.g., obtained following administration to the subject of a radiopharmaceutical, such as 99mTc-MDP), PET (e.g., obtained following administration to the subject of a radiopharmaceutical, such as [18F]DCFPyL, [68Ga]PSMA-11, [18F] PSMA-1007, rhPSMA-7.3 (18F), [18F]-JK-PSMA-7, etc.), or SPECT (e.g., obtained following administration to the subject of a radiopharmaceutical, such as a 99mTc-labeled PSMA binding agent)].

In certain embodiments, the plurality of medical images comprise one or more composite images, each comprising an anatomical and a nuclear medicine pair (e.g., overlaid/co-registered with each other; e.g., having been acquired for the subject at a substantially same time) (e.g., one or more PET/CT images).

In certain embodiments, the plurality of medical images are or comprises a time series of medical images, each medical image of the time series associated with and having been acquired at a different particular time.

In certain embodiments, the time series of medical images comprises a first medical image acquired before administering (e.g., one or more cycles of) a particular therapeutic agent [e.g., a PSMA binding agent (e.g., PSMA-617; e.g., PSMA I&T); e.g., a radiopharmaceutical; e.g., a radionuclide-labeled PSMA binding agent (e.g., 177Lu-PSMA-617; e.g., 177Lu-PSMA I&T)] to the subject and a second medical image acquired after administering (e.g., the one or more cycles of) the particular therapeutic agent to the subject.

In certain embodiments, the method comprises classifying the subject as a responder and/or a non-responder to the particular therapeutic agent based on the values of one or more metrics determined at step (d).

In certain embodiments, step (a) comprises generating each hotspot map by (e.g., automatically) segmenting at least a portion of the corresponding medical image (e.g., a sub-image thereof, such as a nuclear medicine image) (e.g., using a second, hotspot segmentation, machine learning module [e.g., wherein the hotspot segmentation machine module comprises a deep learning network (e.g., a Convolutional Neural Network (CNN))]).

In certain embodiments, each hotspot map comprises, for each of at least a portion of the hotspots identified therein, one or more labels identifying one or more assigned anatomical regions and/or lesion sub-types (e.g., a miTNM classification label).

In certain embodiments, the plurality of hotspot maps comprises (i) a first hotspot map corresponding to a first medical image (e.g., and identifying a first set of one or more hotspots therein) and (ii) a second hotspot map corresponding to a second medical image (e.g., and identifying a second set of one or more hotspots therein); the plurality of 3D anatomical segmentation maps comprises (i) a first 3D anatomical segmentation map identifying the set of organ regions within the first medical image and (ii) a second 3D anatomical segmentation map identifying the set of organ regions within the second medical image; and step (c) comprises registering (i) the first hotspot map with (ii) the second hotspot map using the first 3D anatomical segmentation map and the second 3D anatomical segmentation map (e.g., to determine one or more registration fields (e.g., a full 3D registration field; e.g., a pointwise registration) using the set of organ regions and/or one or more subsets thereof as landmarks within the first and second 3D anatomical segmentation maps and using the one or more determined registration fields to co-register the first and second hotspot maps).

In certain embodiments, step (c) comprises: determining, for a group of two or more hotspots, each a member of a different hotspot map and identified within a different medical image, values of one or more lesion correspondence metrics (e.g., a volume overlap; e.g., a center of mass distance; e.g., a lesion type match); and determining the two or more hotspots of the group to represent a same particular underlying physical lesion based on the values of the one or more lesion correspondence metrics, thereby including the two or more hotspots of the group in one of the one or more lesion correspondences.

In certain embodiments, step (d) comprises determining one, two, or all three of (i), (ii), and (iii) as follows: (i) a change in the number of identified lesions (ii) a change in an overall volume of identified lesions (e.g., a change in the sum of the volumes of each identified lesion), and (iii) a change in PSMA (e.g., lesion index) weighted total volume (e.g., a sum of the products of lesion index and lesion volume for all lesions in a region of interest) [e.g., wherein the change identified in step (b) is used to identify (1) a disease status [e.g., progression, regression, or no change], (2) make a treatment management decision [e.g. active surveillance, prostatectomy, anti-androgen therapy, prednisone, radiation, radio-therapy, radio-PSMA therapy, or chemotherapy], or (3) treatment efficacy (e.g. wherein the subject has begun treatment or has continued treatment with a medicament or other therapy following an initial set of images in the time series of medical images)].

In certain embodiments, the method comprises determining (e.g., based on values of the one or more metrics; e.g., at step (d)) values of one or more prognostic metrics indicative of disease state/progression and/or treatment [e.g., determining an expected overall survival (OS) for the subject (e.g., a predicted number of months)].

In certain embodiments, the method comprises using values of the one or more metrics (e.g., a change in tumor volume, SUV mean, SUV max, PSMA score, number of new lesions, number of disappeared lesions, total number of tracked lesions) as inputs to a prognostic model (e.g., a statistical model, such as a regression; e.g., a classification model, whereby a patient is assigned to a particular class based on a comparison of the one or more patient index values with one or more thresholds; e.g., a machine learning model, where the values of the one or more patient indices are received as input) that generates, as output, an expectation value and/or range (e.g., a class) indicative of a likely value of a particular patient outcome (e.g., a time, e.g., in number of months, representing an expected survival, time to progression, time to radiographic progression, etc.).

In certain embodiments, the method comprises using values of the one or more metrics (e.g., a change in tumor volume, SUV mean, SUV max, PSMA score, number of new lesions, number of disappeared lesions, total number of tracked lesions) as inputs to a response model (e.g., a statistical model, such as a regression; e.g., a classification model, whereby a patient is assigned to a particular class based on a comparison of the one or more patient index values with one or more thresholds; e.g., a machine learning model, where the values of the one or more patient indices are received as input) that generates, as output, a classification (e.g., a binary classification) indicative of a patient response to treatment.

In certain embodiments, the method comprises using values of the one or more metrics (e.g., a change in tumor volume, SUV mean, SUV max, PSMA score, number of new lesions, number of disappeared lesions, total number of tracked lesions) as inputs to a predictive model (e.g., a statistical model, such as a regression; e.g., a classification model, whereby a patient is assigned to a particular class based on a comparison of the one or more patient index values with one or more thresholds; e.g., a machine learning model, where the values of the one or more patient indices are received as input) that generates, as output, an eligibility score for each of one or more treatment options (e.g., Abiraterone, Enzalutamide, Apalutamide, Darolutamide, Sipuleucel-T, Ra223, Docetaxel, Carbazitaxel, Pembrolizumab, Olaparib, Rucaparib, 177Lu-PSMA-617, etc.) and/or classes of therapeutics [e.g., androgen biosynthesis inhibitors (e.g., Abiraterone), androgen receptor inhibitors (e.g., Enzalutamide, Apalutamide, Darolutamide), a cellular immunotherapy (e.g., Sipuleucel-T), internal radiotherapy treatment (Ra223), antineoplastics (e.g., Docetaxel, Carbazitaxel), immune checkpoint inhibitor (Pembrolizumab), PARP inhibitors (e.g., Olaparib, Rucaparib), PSMA binding agent], wherein the eligibility score for a particular treatment option and/or therapeutic class indicates a prediction of whether the patient will benefit from the particular treatment and/or therapeutic class.

In another aspect, the invention is directed to a method for analyzing a plurality of medical images of a subject, the method comprising: (a) obtaining (e.g., receiving and/or accessing, and/or generating), by a processor of a computing device, a first 3D hotspot map for the subject; (b) obtaining (e.g., receiving and/or accessing, and/or generating), by the processor, a first 3D anatomical segmentation map associated with the first 3D hotspot map; (c) obtaining (e.g., receiving and/or accessing, and/or generating), by the processor, a second 3D hotspot map for the subject; (d) obtaining (e.g., receiving and/or accessing, and/or generating), by the processor, a second 3D anatomical segmentation map associated with the second 3D hotspot map; (e) determining, by the processor, a registration field (e.g., a full 3D registration field; e.g., a pointwise registration) using/based on the first 3D anatomical segmentation map and the second 3D anatomical segmentation map; (f) registering, by the processor, the first 3D hotspot map and the second 3D hotspot map, using the determined registration field, thereby generating a co-registered pair of 3D hotspot maps; (g) determining, by the processor, an identification one or more lesion correspondences using the co-registered pair of 3D hotspot maps; and (h) storing and/or providing, by the processor, the identification of the one or more lesion correspondences for display and/or further processing.

In another aspect, the invention is directed to a method for analyzing a plurality of medical images of a subject (e.g., to evaluate disease state and/or progression within the subject), the method comprising: (a) receiving and/or accessing, by a processor of a computing device, the plurality of medical images of the subject; (b) for each particular one (medical image) of the plurality of medical images, determining, by the processor, using a machine learning module [e.g., a deep learning network (e.g., a Convolutional Neural Network (CNN))], a corresponding 3D anatomical segmentation map that identifies a set of organ regions [e.g., representing soft tissue and/or bone structures within the subject (e.g., one or more of a cervical spine; thoracic spine; lumbar spine; left and right hip bones, sacrum and coccyx; left side ribs and left scapula; right side ribs and right scapula; left femur; right femur; skull, brain and mandible)] within the particular medical image, thereby generating a plurality of 3D anatomical segmentation maps; (c) determining, by the processor, using the plurality of 3D anatomical segmentation maps, one or more registration fields (e.g., a full 3D registration field; e.g., a pointwise registration) and applying the one or more registration fields to register the plurality of medical images, thereby creating a plurality of registered medical images; (d) determining, by the processor, for each particular one of the plurality of registered medical images, a corresponding registered 3D hotspot map identifying one or more hotspots (e.g., representing potential underlying physical lesions within the subject) within the particular registered medical image, thereby creating a plurality of registered 3D hotspot maps; (e) determining, by the processor, using the plurality of 3D registered hotspot maps, an identification of one or more lesion correspondences, each (lesion correspondence) identifying two or more corresponding hotspots within different medical images and determined (e.g., by the processor) to represent a same underlying physical lesion within the subject; and (f) determining, by the processor, based on the plurality of 3D hotspot maps and the identification of the one or more lesion correspondences, values of one or more metrics {e.g., one or more hotspot quantification metrics and/or changes therein [e.g., that quantify a change in properties, such as volume, radiopharmaceutical uptake, shape, etc. of individual hotspots and/or the underlying physical lesions that they represent (e.g., over time/between multiple medical images)]; e.g., patient indices (e.g., that that measure overall disease burden and/or state and/or risk for a subject) and/or changes thereof; e.g., values classifying a patient (e.g., as belonging to and/or having a particular disease state, progression, etc. category) e.g., prognostic metrics [e.g., indicative of and/or which quantify one or more clinical outcomes (e.g., a disease state, progression, likely survival, treatment efficacy, and the like) (e.g., overall survival); e.g., predictive metrics (e.g., indicative of a predicted response to therapy and/or other clinical outcome)}.

In another aspect, the invention is directed to a method for analyzing a plurality of medical images of a subject, the method comprising: (a) obtaining (e.g., receiving and/or accessing, and/or generating), by a processor of a computing device, a first 3D anatomical image (e.g., a CT, X-Ray, MRI, etc.) and a first 3D functional image [e.g., a nuclear medicine image (e.g., PET, SPECT, etc.)] of the subject; (b) obtaining (e.g., receiving and/or accessing, and/or generating), by the processor, a second 3D anatomical image and a second 3D functional image of the subject; (c) obtaining (e.g., receiving and/or accessing, and/or generating), by the processor, a first 3D anatomical segmentation map based on (e.g., using) the first 3D anatomical image; (d) obtaining (e.g., receiving and/or accessing, and/or generating), by the processor, a second 3D anatomical segmentation map based on (e.g., using) the second 3D anatomical image; (e) determining, by the processor, a registration field (e.g., a full 3D registration field; e.g., a pointwise registration) using/based on the first 3D anatomical segmentation map and the second 3D anatomical segmentation map; (f) registering, by the processor, the second 3D functional image to (align it with) first 3D functional image using the registration field, thereby generating a registered version of the second 3D functional image; (g) obtaining, by the processor a first 3D hotspot map associated with the first functional image; (h) determining, by the processor, a second 3D hotspot map using the registered version of the second 3D functional image, the second 3D hotspot map thereby being registered with the first 3D hotspot map; (i) determining, by the processor, an identification one or more lesion correspondences using the first 3D hotspot map and the second 3D hotspot map registered thereto; and (j) storing and/or providing, by the processor, the identification of the one or more lesion correspondences for display and/or further processing.

In another aspect, the invention is directed to a method for evaluating efficacy of an intervention, the method comprising: (a) for each particular subject of a test population (e.g., comprising a plurality of subjects, e.g., enrolled in a clinical trial) presenting and/or at risk for a particular disease (e.g., prostate cancer (e.g., metastatic castration resistant prostate cancer)), performing the method of any one of the preceding claims for a plurality of medical images of the particular patient, wherein the plurality of medical images for the particular patient comprises a time series of medical images obtained over a time period spanning (e.g., before, during, and/or after) an intervention under test and the one or more risk indices comprise one or more endpoints indicative of a patient response to the intervention under test, thereby determining a plurality of values of each of the one or more endpoints across the test population; and (b) determining an efficacy of the intervention under test based the values of the one or more endpoints across the test population.

In another aspect, the invention is directed to a method for treating a subject having and/or at risk for a particular disease (e.g., prostate cancer (e.g., metastatic castration resistant prostate cancer)), the method comprising: administering a first cycle of a therapeutic agent to the subject; and administering a second cycle of the therapeutic agent to the subject based on the subject having been imaged (e.g., before and/or during and/or after the first cycle of the therapeutic agent) and identified as a responder to the therapeutic agent using methods described in any one of the aspects and embodiments described herein, for example at paragraphs above (e.g., paragraphs [0003]-[0052]) (e.g., the subject having been identified/classified as a responder based on the values of the one or more risk indices determined using the methods described in any one of the aspects and embodiments described herein, for example at paragraphs above (e.g., paragraphs [0003]-[0052])).

In another aspect, the invention is directed to a method for treating a subject having and/or at risk for a particular disease (e.g., prostate cancer (e.g., metastatic castration resistant prostate cancer)), the method comprising: administering a cycle of a first therapeutic agent to the subject; and administering a cycle of a second therapeutic agent to the subject based on the subject having been imaged (e.g., before and/or during and/or after the cycle of the first therapeutic agent) and identified as a non-responder to the first therapeutic agent using methods described in any one of the aspects and embodiments described herein, for example at paragraphs above (e.g., paragraphs [0003]-[0052]) (e.g., the subject having been identified/classified as a non-responder based on the values of the one or more risk indices determined using methods described in any one of the aspects and embodiments described herein, for example at paragraphs above (e.g., paragraphs [0003]-[0052])) (e.g., thereby moving the subject to a potentially more effective therapy).

In another aspect, the invention is directed to a method for treating a subject having and/or at risk for a particular disease (e.g., prostate cancer (e.g., metastatic castration resistant prostate cancer)), the method comprising: administering a cycle of a therapeutic agent to the subject; and discontinuing administration of the therapeutic agent to the subject based on the subject having been imaged (e.g., before and/or during and/or after the cycle of the first therapeutic agent) and identified as a non-responder to the therapeutic agent using methods described in any one of the aspects and embodiments described herein, for example at paragraphs above (e.g., paragraphs [0003]-[0052]) (e.g., the subject having been identified/classified as a non-responder based on the values of the one or more risk indices determined using methods described in any one of the aspects and embodiments described herein, for example at paragraphs above (e.g., paragraphs [0003]-[0052])) (e.g., thereby moving the subject to a potentially more effective therapy).

In another aspect, the invention is directed to a method of automated or semi-automated whole-body evaluation of a subject with metastatic prostate cancer [e.g., metastatic castration-resistant prostate cancer (mCRPC) or metastatic hormone-sensitive prostate cancer (mHSPC)] to assess disease progression and/or treatment efficacy, the method comprising: (a) receiving, by a processor of a computing device, a first prostate-specific membrane antigen (PSMA) targeting positron emission tomography (PET) image (the first PSMA-PET image) of the subject and a first 3D anatomical image [e.g., a computed tomography (CT) image; e.g., a magnetic resonance image (MRI)] of the subject, wherein the first 3D anatomical image of the subject is obtained simultaneously with or immediately subsequent to or immediately prior to (e.g., on the same date as) the first PSMA PET image such that the first 3D anatomical image and the first PSMA PET image correspond to a first date, and wherein the images depict a large enough area of the subject's body to cover regions of the body to which the metastatic prostate cancer has spread (e.g., full torso images or whole-body images that cover multiple organs) {e.g., wherein the PSMA-PET images are obtained using PYLARIFY®, F-18 piflufolastat PSMA (i.e., 2-(3-{1-carboxy-5-[(6-[18F]fluoro-pyridine-3-carbonyl) amino]-pentyl}ureido)-pentanedioic acid, aka [18F]F-DCFPyL), or Ga-68 PSMA-11, or other radiolabeled prostate-specific membrane antigen inhibitor imaging agent}; (b) receiving, by the processor, a second PSMA-PET image of the subject and a second 3D anatomical image of the subject, both obtained on a second date subsequent to the first date; (c) automatically determining, by the processor, a registration field (e.g., a full 3D registration field; e.g., a pointwise registration) using landmarks automatically identified within the first and second 3D anatomical images (e.g., identified regions representing one or more of a cervical spine; thoracic spine; lumbar spine; left and right hip bones, sacrum and coccyx; left side ribs and left scapula; right side ribs and right scapula; left femur; right femur; skull, brain and mandible), and using, by the processor, the determined registration field to align the first and second PSMA-PET images [e.g., either before or after segmentation of the CT and/or PSMA-PET images to identify boundaries of organs and/or bones, and either before or after automatic hotspot (e.g., lesion) detection from the PSMA-PET images]; and (d) using the thusly aligned first and second PSMA-PET images to automatically detect (e.g., staging and/or quantifying), by the processor, a change in (e.g., a progression of or remission of) the disease from the first date to the second date [e.g., automatically identifying, and/or identifying as such (e.g., tagging, labelling), one or both of (i) and (ii) as follows: (i) a change in the number of lesions {e.g., one or more new lesions (e.g., organ-specific lesions), or an elimination of one or more lesions (e.g., organ-specific)}, and (ii) a change in tumor size {e.g., an increase of tumor size (PSMA-VOL increase/decrease), e.g., total tumor size, or a decrease of tumor size (PSMA-VOL decrease)}{e.g., a change in volume of each of one or more specific lesions, or a change in overall volume of a specific type of lesions (e.g., organ-specific tumor), or a change in total volume of identified lesions}].

In certain embodiments, the method comprises one or more members selected from the group consisting of lesion location assignment, tumor staging, nodal staging, distant metastasis staging, assessment of intraprostatic lesions, and determination of PSMA-expression score.

In certain embodiments, the subject has administered to them a therapy {e.g., a hormone therapy, chemotherapy, and/or radiotherapy, e.g., androgen ablation therapy, e.g., a 177Lu-containing compound, e.g., a 177Lu-PSMA radioligand therapy, e.g., 177Lu-PSMA-617, e.g., lutetium Lu 177 vipivotide tetraxetan (Pluvicto), e.g., cabazitaxel} for treatment of the metastatic prostate cancer at one or more times from the first date to the second date (after the first images are obtained and before the second images are obtained), such that the method is used to assess treatment efficacy.

In certain embodiments, the method further comprising obtaining a one or more further PSMA PET images and 3D anatomical images of the subject subsequent to the second date, aligning the further PSMA PET image(s) using corresponding 3D anatomical image(s), and using the aligned further PSMA PET image(s) to assess the disease progression and/or treatment efficacy.

In certain embodiments, the method further comprising determining and rendering, by the processor, a predicted PSMA-PET image depicting a predicted progression (or remission) of disease to a future date (e.g., a future date that is later than the second date or any other subsequent date on which PSMA-PET images have been obtained) based at least in part on the detected change in the disease from the first date to the second date.

In another aspect, the invention is directed to a method of quantifying and reporting disease (e.g., tumor) burden for a patient having and/or at risk for cancer, the method comprising: (a) obtaining, by a processor of a computing device, a medical image of the patient; (b) detecting, by the processor, a one or more (e.g., a plurality of) hotspots within the medical image, each hotspot corresponding to (e.g., being or comprising) a particular 3D volume [e.g., a 3D hotspot volume; e.g., wherein voxels of the 3D hotspot volume have an elevated intensity (e.g., and/or are otherwise indicative or increased radiopharmaceutical uptake) relative to their surroundings] within the medical image and representing a potential underlying physical lesion within the subject; (c) for each particular lesion class of a plurality of lesion classes representing specific tissue regions and/or lesion sub-types: identifying, by the processor, a corresponding subset of the one or more hotspot(s) as belonging to the particular lesion class (e.g., based on a determination made, by the processor, that the hotspot represents an underlying physical lesion located within a particular tissue region and/or belonging to a particular lesion sub-type that the particular lesion class represents); and determining, by the processor, values of one or more patient index(indices) that quantify disease (e.g., tumor) burden within and/or associated with the particular lesion class based on the corresponding subset of hotspots; and (d) causing, by the processor, display of a graphical representation of the patient index values computed for each of the plurality of lesion classes (e.g., a summary table listing each lesion class and, for each lesion class, the computed patient index values), thereby providing a user with a graphical report summarizing tumor burden, within specific tissue regions and/or, associated with specific lesion sub-types.

In certain embodiments, the plurality of lesion classes comprise one or more of the following: (i) a local tumor class (e.g., a “T” or “miT” class) that identifies, and for which the corresponding subset of hotspots represents, potential lesions and/or portions thereof that are located within one or more local tumor-associated tissue regions associated with and/or adjacent to a localized (e.g., primary) tumor site within the patient [e.g., wherein the cancer is prostate cancer and the one or more local tumor-associated tissue regions comprise a prostate and, optionally, one or more adjacent structures (e.g., seminal vesicle(s), an external sphincter, a rectum, a bladder, levator muscles, and/or a pelvic wall); e.g., wherein the cancer is breast cancer and the one or more local tumor-associated tissue region(s) comprise a breast; e.g., wherein the cancer is colorectal cancer and the one or more local tumor-associated tissue region(s) comprise a colon; e.g., wherein the cancer is lung cancer and one or more local tumor-associated tissue region(s) comprise a lung]; (ii) a regional node class (e.g., a “N” or “miN” class) that identifies, and for which the corresponding subset of hotspots represents, potential lesions located within regional lymph nodes that are adjacent and/or in proximity to an original (e.g., primary) tumor site [e.g., wherein the cancer is prostate cancer and the regional lymph node class identifies hotspots representing lesions located within one or more pelvic lymph nodes (e.g., an internal iliac, an external iliac, an obturator, a presacral node, or other pelvic lymph node)]; and (iii) one or more (e.g., distant) metastatic tumor classes (e.g., one or more “M” or “miM” class(es)) that identify, and for which the corresponding subset of hotspots represents, potential metastases (e.g., lesions having spread outside the original (e.g., primary) tumor site) and/or sub-types thereof [e.g., wherein the cancer is prostate cancer and the one or more metastatic tumor classes identify hotspots that represent potential metastatic lesions located outside a pelvic region (e.g., as defined by the pelvic brim, e.g., according to an American Joint Committee on Cancer staging manual) of the patient].

In certain embodiments, the one or more metastatic tumor classes comprise one or more of the following: a distant lymph node metastases class (e.g., a “Ma” or “miMa” class) that identifies, and for which the corresponding subset of hotspots represents, potential lesions having metastasized to distant lymph nodes [e.g., wherein the cancer is prostate cancer and the distant lymph node region class identifies hotspots that represent lesions located within extrapelvic (e.g., outside a pelvic region) lymph nodes (e.g., a common iliac, retroperitoneal lymph nodes, supradiaphragmatic lymph nodes, inguinal and other extraplevic lymph nodes)]; a distant bone metastases class (e.g., a “Mb” or “miMb” class) that identifies, and for which the corresponding subset of hotspots represents, potential lesions located within one or more bones (e.g., distant bones) of the patient; and a visceral (also referred to as distant soft-tissue) metastases class (e.g., a “Mc” or “miMc” class) that identifies, and for which the corresponding subset of hotspots represents, potential lesions located within one or more organs or other non-lymph soft tissue regions outside the local tumor-associated tissue regions (e.g., wherein the cancer is prostate cancer and the visceral metastases class identifies hotspots representing potential lesions located in extrapelvic organs, such as a brain, lungs, a liver, a spleen, and kidneys, of the patient).

In certain embodiments, step (c) comprises determining, for each particular lesion class, a value of one or more of the following patient index(indices): a lesion count that quantifies a number of (e.g., distinct) lesions represented by the subset of hotspots corresponding to the particular lesion class (e.g., computed as a number of hotspots within the corresponding subset); a maximum uptake value that quantifies a maximum uptake within the corresponding set of hotspots (e.g., computed as a maximum individual voxel intensity over all voxels within hotspot volumes of the corresponding subset; e.g., according to equation (13a)); a mean uptake value that quantifies an overall mean uptake within the corresponding subset of hotspots (e.g., computed as an overall mean intensity over all voxels within the (total combined) hotspot volume of the corresponding subset; e.g., according to equation (13b)); a total volume lesion volume that quantifies a total volume of lesions belonging to the particular lesion class (e.g., computed as sum of all individual lesion (e.g., hotspot) volumes of the corresponding subset; e.g., according to equation (13c)); and an intensity weighted tumor volume (ILTV) score (e.g., an aPSMA score) computed as a weighted sum of all individual lesion volumes weighted (e.g., multiplied by) a measure of their intensity [e.g., wherein the measure of their intensity is a lesion index that quantifies hotspot intensity on a standardized scale based on a comparison with one or more reference intensities indicative of physiological (e.g., normal, non-cancer associated) radiopharmaceutical uptake within one or more corresponding reference tissue regions, such as an aorta portion and a liver] [e.g., computed according to equation (13d)].

In certain embodiments, the method comprises determining, for each of the lesion classes, an alpha-numeric code classifying an overall burden within the particular lesion class (e.g., an miTNM staging code that indicates (i) the particular lesion class along with (ii) one or more numbers and/or numbers that indicate a particular number, size, spatial extent, spatial pattern, and/or sub-location of hotspots of the corresponding subset and, in turn, the underlying physical lesions they represent) and, optionally, at step (e), causing generation and/or display of a representation of the alpha-numeric code for each particular lesion class.

In certain embodiments, the method further comprises determining, based on the plurality of lesion classes and their corresponding subsets of hotspots, an overall disease stage for the patient (e.g., an alphanumeric code) indicative of an overall disease status and/or burden for the patient and causing, by the processor, rendering a graphical representation of the overall disease stage (e.g., an alphanumeric code) for inclusion within the report.

In certain embodiments, the method further comprises: determining, by the processor, one or more reference intensity values, each indicative of physiological (e.g., normal, non-cancer-related) uptake of radiopharmaceutical within a particular reference tissue region (e.g., an aorta portion; e.g., a liver) within the patient and computed based on intensities of image voxels within a corresponding reference volume identified within the medical image; and at step (d), causing, by the processor, rendering of a representation (e.g., a table) of the one or more reference intensity values for inclusion within the report.

In another aspect, the invention is directed to a method of characterizing and reporting individual lesions detected based on an imaging assessment of a patient having and/or at risk for cancer, the method comprising: (a) obtaining, by a processor of a computing device, a medical image of the patient; (b) detecting, by the processor, a set of one or more (e.g., a plurality of) hotspots within the medical image, each hotspot of the set corresponding to (e.g., being or comprising) a particular 3D volume [e.g., a 3D hotspot volume; e.g., wherein voxels of the 3D hotspot volume have an elevated intensity (e.g., and/or are otherwise indicative or increased radiopharmaceutical uptake) relative to their surroundings] within the medical image and representing a potential underlying physical lesion within the subject; (c) assigning, by the processor, one or more lesion class labels to each of the one or more hotspots of the set, each lesion class label classes representing a specific tissue regions and/or lesion sub-types and identifying the hotspot as representing a potential lesion located within the specific tissue region and/or belonging to the lesion sub-type; (d) computing, by the processor, for each particular one of one or more individual hotspot quantification metrics, a value of the particular individual hotspot quantification metrics for each individual hotspot of the set; and (e) causing, by the processor, display of a graphical representation comprising, for each particular hotspot of at least a portion of the hotspots of the set, an identification of the particular hotspot (e.g., a row in a table, and, optionally, an alphanumeric identification, such as a number, identifying the particular hotspot) along with the one or more lesion class labels assigned to the particular hotspot and the values of the one or more individual hotspot quantification metrics computed for the particular hotspot [e.g., a summary table (e.g., a scrollable summary table) listing each individual hotspot as a row and the assigned lesion classes and hotspot quantification metrics column-wise].

In certain embodiments, the lesion class labels comprise labels representing one or more of the following: (i) a local tumor class (e.g., a “T” or “miT” class) that identifies, and for which the corresponding subset of hotspots represents, potential lesions and/or portions thereof that are located within one or more local tumor-associated tissue regions associated with and/or adjacent to a localized (e.g., primary) tumor site within the patient [e.g., wherein the cancer is prostate cancer and the one or more local tumor-associated tissue regions comprise a prostate and, optionally, one or more adjacent structures (e.g., seminal vesicle(s), an external sphincter, a rectum, a bladder, levator muscles, and/or a pelvic wall); e.g., wherein the cancer is breast cancer and the one or more local tumor-associated tissue region(s) comprise a breast; e.g., wherein the cancer is colorectal cancer and the one or more local tumor-associated tissue region(s) comprise a colon; e.g., wherein the cancer is lung cancer and one or more local tumor-associated tissue region(s) comprise a lung]; (ii) a regional node class (e.g., a “N” or “miN” class) that identifies, and for which the corresponding subset of hotspots represents, potential lesions located within regional lymph nodes that are adjacent and/or in proximity to an original (e.g., primary) tumor site [e.g., wherein the cancer is prostate cancer and the regional lymph node class identifies hotspots representing lesions located within one or more pelvic lymph nodes (e.g., an internal iliac, an external iliac, an obturator, a presacral node, or other pelvic lymph node)]; and (iii) one or more (e.g., distant) metastatic tumor classes (e.g., one or more “M” or “miM” class(es)) that identify, and for which the corresponding subset of hotspots represents, potential metastases (e.g., lesions having spread outside the original (e.g., primary) tumor site) and/or sub-types thereof [e.g., wherein the cancer is prostate cancer and the one or more metastatic tumor classes identify hotspots that represent potential metastatic lesions located outside a pelvic region (e.g., as defined by the pelvic brim, e.g., according to an American Joint Committee on Cancer staging manual) of the patient].

In certain embodiments, the one or more metastatic tumor classes comprise one or more of the following: a distant lymph node metastases class (e.g., a “Ma” or “miMa” class) that identifies, and for which the corresponding subset of hotspots represents, potential lesions having metastasized to distant lymph nodes [e.g., wherein the cancer is prostate cancer and the distant lymph node region class identifies hotspots that represent lesions located within extrapelvic (e.g., outside a pelvic region) lymph nodes (e.g., a common iliac, retroperitoneal lymph nodes, supradiaphragmatic lymph nodes, inguinal and other extraplevic lymph nodes)]; a distant bone metastases class (e.g., a “Mb” or “miMb” class) that identifies, and for which the corresponding subset of hotspots represents, potential lesions located within one or more bones (e.g., distant bones) of the patient; and a visceral (also referred to as distant soft-tissue) metastases class (e.g., a “Mc” or “miMc” class) that identifies, and for which the corresponding subset of hotspots represents, potential lesions located within one or more organs or other non-lymph soft tissue regions outside the local tumor-associated tissue regions (e.g., wherein the cancer is prostate cancer and the visceral metastases class identifies hotspots representing potential lesions located in extrapelvic organs, such as a brain, lungs, a liver, a spleen, and kidneys, of the patient).

In certain embodiments, the lesion class labels comprise one or more tissue labels that identifies a particular organ or bone in which the (lesion represented by the hotspot) is determined to be located (e.g., based on a comparison of the hotspot with an anatomical segmentation map) (e.g., one or more of the organs or bone regions listed in Table 1)

In certain embodiments, the one or more individual hotspot quantification metrics include one or more of the following: a maximum intensity (e.g., SUV-max) (e.g., determined according to any one of equations (1a), (1b), or (1c)), a peak intensity (e.g., SUV-peak) (e.g., determined according to any one of equations (3a), (3b), or (3c)), a mean intensity (e.g., SUV-mean) (e.g., determined according to any one of equations (2a), (2b), (2c)), a lesion volume (e.g., determined according to any one of equations (5a) or (5b) and a lesion index (e.g., that measures intensity of the hotspot on a standardized scale) (e.g., determined according to equation (4)).

In another aspect, the invention is directed to a method of quantifying and reporting disease (e.g., tumor) progression and/or risk over time for a patient having and/or at risk for cancer, the method comprising: (a) obtaining, by a processor of a computing device, a plurality of medical images of the patient, each medical image representing a scan of the patient obtained at a particular time (e.g., a longitudinal dataset); (b) for each particular one of the plurality of medical images, detecting, by the processor, a corresponding set of one or more (e.g., a plurality of) hotspots within the particular medical image, each hotspot corresponding to (e.g., being or comprising) a particular 3D volume [e.g., a 3D hotspot volume; e.g., wherein voxels of the 3D hotspot volume have an elevated intensity (e.g., and/or are otherwise indicative or increased radiopharmaceutical uptake) relative to their surroundings] within the medical image and representing a potential underlying physical lesion within the subject; (c) for each particular one of one or more (e.g., overall) patient index(indices) that measure (e.g., quantify) overall disease (e.g., tumor) burden within a patient at a particular time, determining, by the processor, a value of the particular (e.g., overall) patient index for each particular medical image of the plurality of medical images based on the corresponding set of hotspots detected for particular medical image, thereby determining, for each particular one of the one or more patient index(indices) a set of values tracking a change in disease burden as measured by to the particular patient index value over time; and (d) causing, by the processor, display of a graphical representation of the set of values for at least a portion (e.g., a particular one, a particular subset) of the one or more of the patient index values, thereby conveying a measure of disease progression over time for the patient.

In certain embodiments, the one or more patient index(indices) comprise: a lesion count that quantifies a number of (e.g., distinct) lesions represented by the set of hotspots corresponding to and detected within a particular medical image (e.g., at a particular point in time) (e.g., computed as a number of hotspots within the corresponding set of hotspots); a maximum uptake value that quantifies a maximum uptake within the corresponding set of hotspots for a particular medical image (e.g., computed as a maximum individual voxel intensity over all voxels within hotspot volumes of the corresponding set of hotspots for the particular medical image; e.g., according to equations (7a) or (7b)); a mean uptake value that quantifies an overall mean uptake within the corresponding set of hotspots (e.g., computed as an overall mean intensity over all voxels within the (total combined) hotspot volume of the corresponding set; e.g., according to equations (10a) or (10b)); a total volume lesion volume that quantifies a total volume of lesions detected within the subject at a particular point in time (e.g., computed as sum of all individual hotspot volumes of the corresponding set of hotspots detected within a particular medical image); and an intensity weighted tumor volume (ILTV) score (e.g., an aPSMA score) computed as a weighted sum of all individual lesion volumes, each individual lesion volume weighted (e.g., multiplied by) a measure of its intensity [e.g., wherein the measure of hotspot intensity is a lesion index that quantifies hotspot intensity on a standardized scale based on a comparison with one or more reference intensities indicative of physiological (e.g., normal, non-cancer associated) radiopharmaceutical uptake within one or more corresponding reference tissue regions, such as an aorta portion and a liver] [e.g., computed according to equation (12)].

In certain embodiments, the method further comprises determining, for each particular medical image of the plurality of medical images, an overall disease stage (e.g., an alphanumeric code) based on the corresponding set of hotspots and indicative of an overall disease status and/or burden for the patient at a particular point in time and causing, by the processor, rendering a graphical representation of the overall disease stages (e.g., alphanumeric codes) at each of point in time.

In certain embodiments, the method further comprises: determining, by the processor, for each of the plurality of medical images, a set of one or more reference intensity values, each indicative of physiological (e.g., normal, non-cancer-related) uptake of radiopharmaceutical within a particular reference tissue region (e.g., an aorta portion; e.g., a liver) within the patient and computed based on intensities of image voxels within a corresponding reference volume identified within the medical image; and causing, by the processor, rendering of a representation (e.g., a table; e.g., a trace in a graph) of the one or more reference intensity values.

In another aspect, the invention is directed to a method for automatically processing 3D images of a subject to determine values of one or more patient index(indices) that measure (e.g., overall) disease burden and/or risk for a subject, the method comprising: (a) receiving, by a processor of a computing device, a 3D functional image of the subject obtained using a functional imaging modality; (b) segmenting, by the processor, a plurality of 3D hotspot volumes within the 3D functional image, each 3D hotspot volume corresponding to a local region of elevated intensity with respect to its surrounding and representing a potential cancerous lesion within the subject, thereby obtaining a set of 3D hotspot volumes; (c) computing, by the processor, for each particular one of one or more individual hotspot quantification metrics, a value of the particular individual hotspot quantification metric for each individual 3D hotspot volume of the set, wherein for a particular individual 3D hotspot volume, each hotspot quantification metric quantifies a property (e.g., intensity, volume, etc.) of the particular 3D hotspot volume and is (e.g., computed as) a particular function of intensities and/or a number of individual voxels within the particular 3D hotspot volume; and (d) determining, by the processor, the values of the one or more patient index(indices), wherein each of at least a portion of the patient indices is associated with one or more specific individual hotspot quantification metrics and is computed using the (e.g., same) particular function with intensities and/or number of voxels within a combined hotspot volume comprising (e.g., formed as a union of) at least a portion (e.g., substantially all; e.g., a particular subset) of the set of 3D hotspot volumes

In certain embodiments, a particular patient index is an overall mean voxel intensity and is computed as an overall mean of voxel intensities located within the combined hotspot volume.

In another aspect, the invention is directed to a method for automatically determining a prognosis for a subject with prostate cancer from one or more medical images of the subject [e.g., one or more PSMA PET images (PET images obtaining upon administering to the subject a PSMA targeting compound) and/or one or more anatomical (e.g., CT) images], the method comprising: (a) receiving and/or accessing, by a processor of a computing device, the one or more images of the subject; (b) automatically determining, by the processor, a quantitative assessment of one or more prostate cancer lesions (e.g., metastatic prostate cancer lesions) from the one or more images [e.g., wherein the quantitative assessment comprises one or more members selected from the group consisting of (i) a molecular imaging TNM (miTNM) lesion type classification for local (T), pelvic-nodal (N), and/or extra-pelvic (M) disease (e.g., miT, miN, miMa (lymph), miMb (bone), miMc (other)); (ii) an indication of lesion location (e.g., prostate, iliac, pelvic bone, rib cage, etc.), (iii) a standard physiological uptake value (SUV) (e.g., SUVmax, SUVpeak, SUVmean), (iv) a total lesion volume, (v) a change in lesion volume (e.g., individual lesion and/or total lesions), and (vi) a computed PSMA (aPSMA) score] (e.g., using one or more of the methods described herein); and (c) automatically determining, from the quantitative assessment in (b), a prognosis for the subject, wherein the prognosis comprises one or more of the following for the subject: (I) an expected survival (e.g., months), (II) an expected time to disease progression, (III) an expected time to radiographic progression, (IV) a risk of concurrent (synchronous) metastases, and (V) a risk of future (metachronous) metastases.

In certain embodiments, the quantitative assessment of the one or more prostate cancer lesions determined in step (b) comprises one or more of the following: (A) a total tumor volume, (B) a change in tumor volume, (C) a total SUV, and (D) a PSMA score, and wherein the prognosis for the subject determined in step (c) comprises one or more of the following: (E) an expected survival (e.g., months), (F) a time to progression, and (G) a time to radiographic progression.

In certain embodiments, the quantitative assessment of the one or more prostate cancer lesions determined in step (b) comprises one or more characteristics of PSMA expression in prostate, and wherein the prognosis for the subject determined in step (c) comprises a risk of concurrent (synchronous) metastases, and/or a risk of future (metachronous) metastases.

In another aspect, the invention is directed to a method for automatically determining a response to a treatment for a subject with prostate cancer from a plurality of medical images of the subject [e.g., one or more PSMA PET images (PET images obtaining upon administering to the subject a PSMA targeting compound) and/or one or more anatomical (e.g., CT) images], the method comprising: (a) receiving and/or accessing, by a processor of a computing device, the plurality of images of the subject, wherein at least a first image of the plurality was obtained prior to an administration of the treatment and at least a second image of the plurality was obtained following administration of the treatment (e.g., after a period of time); (b) automatically determining, by the processor, a quantitative assessment of one or more prostate cancer lesions (e.g., metastatic prostate cancer lesions) from the images [e.g., wherein the quantitative assessment comprises one or more members selected from the group consisting of (i) a molecular imaging TNM (miTNM) lesion type classification for local (T), pelvic-nodal (N), and/or extra-pelvic (M) disease (e.g., miT, miN, miMa (lymph), miMb (bone), miMc (other)); (ii) an indication of lesion location (e.g., prostate, iliac, pelvic bone, rib cage, etc.), (iii) a standard physiological uptake value (SUV) (e.g., SUVmax, SUVpeak, SUVmean), (iv) a total lesion volume, (v) a change in lesion volume (e.g., individual lesion and/or total lesions), and (vi) a computed PSMA (aPSMA) score] (e.g., using one or more of the methods described herein) (e.g., wherein the quantitative assessment comprises Response Evaluation Criteria in PSMA-imaging (RECIP) criteria and/or PSMA PET Progression (PPP) criteria); and (c) automatically determining, from the quantitative assessment in (b), whether the subject is responding to the treatment (e.g., a yes/no) and/or a degree to which the subject is responding to the treatment (e.g., a numerical value or classification).

In another aspect, the invention is directed to a method for automatically identifying whether a subject with prostate cancer (e.g., metastatic prostate cancer) is likely to benefit from a particular treatment for prostate cancer using a plurality of medical images of the subject [e.g., one or more PSMA PET images (PET images obtaining upon administering to the subject a PSMA targeting compound) and/or one or more anatomical (e.g., CT) images], the method comprising: (a) receiving and/or accessing, by a processor of a computing device, the plurality of images of the subject; (b) automatically determining, by the processor, a quantitative assessment of one or more prostate cancer lesions (e.g., metastatic prostate cancer lesions) from the images [e.g., wherein the quantitative assessment comprises one or more members selected from the group consisting of (i) a molecular imaging TNM (miTNM) lesion type classification for local (T), pelvic-nodal (N), and/or extra-pelvic (M) disease (e.g., miT, miN, miMa (lymph), miMb (bone), miMc (other)); (ii) an indication of lesion location (e.g., prostate, iliac, pelvic bone, rib cage, etc.), (iii) a standard physiological uptake value (SUV) (e.g., SUVmax, SUVpeak, SUVmean), (iv) a total lesion volume, (v) a change in lesion volume (e.g., individual lesion and/or total lesions), and (vi) a computed PSMA (aPSMA) score] (e.g., using one or more of the methods described herein) (e.g., wherein the quantitative assessment comprises Response Evaluation Criteria in PSMA-imaging (RECIP) criteria and/or PSMA PET Progression (PPP) criteria); and (c) automatically determining, from the quantitative assessment in (b), whether the subject is likely to benefit from the particular treatment for prostate cancer [e.g., determining for the subject an eligibility score for one or more particular treatments and/or a class of treatments, e.g., a particular radioligand therapy, e.g., lutetium vipivotide tetraxetan (Pluvicto®)].

In another aspect, the invention is directed to a system for automatically processing 3D images of a subject to determine values of one or more patient index(indices) that measure (e.g., overall) disease burden and/or risk for a subject, the system comprising: a processor of a computing device; and memory having instructions stored thereon, wherein the instructions, when executed by the processor, cause the processor to: (a) receive a 3D functional image of the subject obtained using a functional imaging modality; (b) segment a plurality of 3D hotspot volumes within the 3D functional image, each 3D hotspot volume corresponding to a local region of elevated intensity with respect to its surrounding and representing a potential cancerous lesion within the subject, thereby obtaining a set of 3D hotspot volumes; (c) compute, for each particular one of one or more individual hotspot quantification metrics, a value of the particular individual hotspot quantification metric for each individual 3D hotspot volume of the set; and (d) determine the values of the one or more patient index(indices), wherein each of at least a portion of the patient indices is associated with one or more specific individual hotspot quantification metrics and is a function of at least a portion (e.g., substantially all; e.g., a particular subset) of the values of the one or more specific individual hotspot quantification metric(s) computed for the set of 3D hotspot volumes.

In certain embodiments, the system has one or more features and/or the instructions cause the processor to perform one or more steps articulated herein (e.g., in paragraphs above, for example at paragraphs [0004]-[0031]).

In another aspect, the invention is directed to a system for automated analysis of a time series of medical images [e.g., three-dimensional images, e.g., nuclear medicine images (e.g., bone scan (scintigraphy), PET, and/or SPECT), e.g., anatomical images (e.g., CT, X-ray, MRI), e.g., combined nuclear medicine and anatomical images (e.g., overlaid)] of a subject, the system comprising: a processor of a computing device; and memory having instructions stored thereon, wherein the instructions, when executed by the processor, cause the processor to: (a) receive and/or access the time series of medical images of the subject; and (b) identify a plurality of hotspots within each of the medical images and determining, by the processor, one, two, or all three of (i), (ii), and (iii) as follows: (i) a change in the number of identified lesions (ii) a change in an overall volume of identified lesions (e.g., a change in the sum of the volumes of each identified lesion), and (iii) a change in PSMA (e.g., lesion index) weighted total volume (e.g., a sum of the products of lesion index and lesion volume for all lesions in a region of interest) [e.g., wherein the change identified in step (b) is used to identify (1) a disease status [e.g., progression, regression, or no change], (2) make a treatment management decision [e.g. active surveillance, prostatectomy, anti-androgen therapy, prednisone, radiation, radio-therapy, radio-PSMA therapy, or chemotherapy], or (3) treatment efficacy (e.g. wherein the subject has begun treatment or has continued treatment with a medicament or other therapy following an initial set of images in the time series of medical images)] [e.g., wherein step (b) comprises using a machine learning module/model].

In another aspect, the invention is directed to a system for analyzing a plurality of medical images of a subject (e.g., to evaluate disease state and/or progression within the subject), the system comprising: a processor of a computing device; and memory having instructions stored thereon, wherein the instructions, when executed by the processor, cause the processor to: (a) receive and/or access the plurality of medical images of the subject and obtaining, by the processor, a plurality of 3D hotspot maps, each corresponding to a particular medical image (of the plurality) and identifying one or more hotspots (e.g., representing potential underlying physical lesions within the subject) within the particular medical image; (b) for each particular one (medical image) of the plurality of medical images, determine, using a machine learning module [e.g., a deep learning network (e.g., a Convolutional Neural Network (CNN))], a corresponding 3D anatomical segmentation map that identifies a set of organ regions [e.g., representing soft tissue and/or bone structures within the subject (e.g., one or more of a cervical spine; thoracic spine; lumbar spine; left and right hip bones, sacrum and coccyx; left side ribs and left scapula; right side ribs and right scapula; left femur; right femur; skull, brain and mandible)] within the particular medical image, thereby generating a plurality of 3D anatomical segmentation maps; (c) determine, using (i) the plurality of 3D hotspot maps and (ii) the plurality of 3D anatomical segmentation maps, an identification of one or more lesion correspondences, each (lesion correspondence) identifying two or more corresponding hotspots within different medical images and determined (e.g., by the processor) to represent a same underlying physical lesion within the subject; and (d) determine, based on the plurality of 3D hotspot maps and the identification of the one or more lesion correspondences, values of one or more metrics {e.g., one or more hotspot quantification metrics and/or changes therein [e.g., that quantify a change in properties, such as volume, radiopharmaceutical uptake, shape, etc. of individual hotspots and/or the underlying physical lesions that they represent (e.g., over time/between multiple medical images)]; e.g., patient indices (e.g., that that measure overall disease burden and/or state and/or risk for a subject) and/or changes thereof; e.g., values classifying a patient (e.g., as belonging to and/or having a particular disease state, progression, etc. category) e.g., prognostic metrics [e.g., indicative of and/or which quantify a likelihood of one or more clinical outcomes (e.g., a disease state, progression, likely survival, treatment efficacy, and the like) (e.g., overall survival); e.g., predictive metrics (e.g., indicative of a predicted response to therapy and/or other clinical outcome)}.

In certain embodiments, the system has one or more features and/or the instructions cause the processor to perform one or more steps articulated herein (e.g., in paragraphs above, for example at paragraphs [0034]-[0048]).

In another aspect, the invention is directed to a system for analyzing a plurality of medical images of a subject, the system comprising: a processor of a computing device; and memory having instructions stored thereon, wherein the instructions, when executed by the processor, cause the processor to: (a) obtain (e.g., receive and/or access, and/or generate) a first 3D hotspot map for the subject; (b) obtain (e.g., receive and/or access, and/or generate) a first 3D anatomical segmentation map associated with the first 3D hotspot map; (c) obtain (e.g., receive and/or access, and/or generate) a second 3D hotspot map for the subject; (d) obtain (e.g., receive and/or access, and/or generate) a second 3D anatomical segmentation map associated with the second 3D hotspot map; (e) determine a registration field (e.g., a 3D registration field and/or a pointwise registration) using/based on the first 3D anatomical segmentation map and the second 3D anatomical segmentation map; (f) register the first 3D hotspot map and the second 3D hotspot map, using the registration field, thereby generating a co-registered pair of 3D hotspot maps; g) determine an identification one or more lesion correspondences using the co-registered pair of 3D hotspot maps; and (h) store and/or provide the identification of the one or more lesion correspondences for display and/or further processing.

In another aspect, the invention is directed to a system for analyzing a plurality of medical images of a subject (e.g., to evaluate disease state and/or progression within the subject), the system comprising: a processor of a computing device; and memory having instructions stored thereon, wherein the instructions, when executed by the processor, cause the processor to: (a) receive and/or access the plurality of medical images of the subject; (b) for each particular one (medical image) of the plurality of medical images, determine, using a machine learning module [e.g., a deep learning network (e.g., a Convolutional Neural Network (CNN))], a corresponding 3D anatomical segmentation map that identifies a set of organ regions [e.g., representing soft tissue and/or bone structures within the subject (e.g., one or more of a cervical spine; thoracic spine; lumbar spine; left and right hip bones, sacrum and coccyx; left side ribs and left scapula; right side ribs and right scapula; left femur; right femur; skull, brain and mandible)] within the particular medical image, thereby generating a plurality of 3D anatomical segmentation maps; (c) determine, using the plurality of 3D anatomical segmentation maps, one or more registration fields (e.g., a full 3D registration field; e.g., a pointwise registration) and applying the one or more registration fields to register the plurality of medical images, thereby creating a plurality of registered medical images; (d) determine, for each particular one of the plurality of registered medical images, a corresponding registered 3D hotspot map identifying one or more hotspots (e.g., representing potential underlying physical lesions within the subject) within the particular registered medical image, thereby creating a plurality of registered 3D hotspot maps; (e) determine, using the plurality of 3D registered hotspot maps, an identification of one or more lesion correspondences, each (lesion correspondence) identifying two or more corresponding hotspots within different medical images and determined (e.g., by the processor) to represent a same underlying physical lesion within the subject; and (e) determine, based on the plurality of 3D hotspot maps and the identification of the one or more lesion correspondences, values of one or more metrics {e.g., one or more hotspot quantification metrics and/or changes therein [e.g., that quantify a change in properties, such as volume, radiopharmaceutical uptake, shape, etc. of individual hotspots and/or the underlying physical lesions that they represent (e.g., over time/between multiple medical images)]; e.g., patient indices (e.g., that that measure overall disease burden and/or state and/or risk for a subject) and/or changes thereof; e.g., values classifying a patient (e.g., as belonging to and/or having a particular disease state, progression, etc. category) e.g., prognostic metrics [e.g., indicative of and/or which quantify one or more clinical outcomes (e.g., a disease state, progression, likely survival, treatment efficacy, and the like) (e.g., overall survival); e.g., predictive metrics (e.g., indicative of a predicted response to therapy and/or other clinical outcome)}.

In another aspect, the invention is directed to a system for analyzing a plurality of medical images of a subject, the system comprising: a processor of a computing device; and memory having instructions stored thereon, wherein the instructions, when executed by the processor, cause the processor to: (a) obtain (e.g., receive and/or access, and/or generate) a first 3D anatomical image (e.g., a CT, X-Ray, MRI, etc.) and a first 3D functional image [e.g., a nuclear medicine image (e.g., PET, SPECT, etc.)] of the subject; (b) obtain (e.g., receive and/or access, and/or generate) a second 3D anatomical image and a second 3D functional image of the subject; (c) obtain (e.g., receive and/or access, and/or generate) a first 3D anatomical segmentation map based on (e.g., using) the first 3D anatomical image; (d) obtain (e.g., receive and/or access, and/or generate) a second 3D anatomical segmentation map based on (e.g., using) the second 3D anatomical image; (e) determine a registration field (e.g., a 3D registration field and/or a pointwise registration) using/based on the first 3D anatomical segmentation map and the second 3D anatomical segmentation map; (f) register the second 3D functional image to (align it with) first 3D functional image using the registration field, thereby generating a registered version of the second 3D functional image; (g) obtain a first 3D hotspot map associated with the first functional image; (h) determine a second 3D hotspot map using the registered version of the second 3D functional image, the second 3D hotspot map thereby being registered with the first 3D hotspot map; (i) determine an identification one or more lesion correspondences using the first 3D hotspot map and the second 3D hotspot map registered thereto; and (j) store and/or provide the identification of the one or more lesion correspondences for display and/or further processing.

In another aspect, the invention is directed to a system of automated or semi-automated whole-body evaluation of a subject with metastatic prostate cancer [e.g., metastatic castration-resistant prostate cancer (mCRPC) or metastatic hormone-sensitive prostate cancer (mHSPC)] to assess disease progression and/or treatment efficacy, the system comprising: a processor of a computing device; and memory having instructions stored thereon, wherein the instructions, when executed by the processor, cause the processor to: (a) receive a first prostate-specific membrane antigen (PSMA) targeting positron emission tomography (PET) image (the first PSMA-PET image) of the subject and a first 3D anatomical image [e.g., a computed tomography (CT) image; e.g., a magnetic resonance image (MRI)] of the subject, wherein the first 3D anatomical image of the subject is obtained simultaneously with or immediately subsequent to or immediately prior to (e.g., on the same date as) the first PSMA PET image such that the first 3D anatomical image and the first PSMA PET image correspond to a first date, and wherein the images depict a large enough area of the subject's body to cover regions of the body to which the metastatic prostate cancer has spread (e.g., full torso images or whole-body images that cover multiple organs) {e.g., wherein the PSMA-PET images are obtained using PYLARIFY®, F-18 piflufolastat PSMA (i.e., 2-(3-{1-carboxy-5-[(6-[18F]fluoro-pyridine-3-carbonyl) amino]-pentyl}ureido)-pentanedioic acid, aka [18F]F-DCFPyL), or Ga-68 PSMA-11, or other radiolabeled prostate-specific membrane antigen inhibitor imaging agent}; (b) receive, a second PSMA-PET image of the subject and a second 3D anatomical image of the subject, both obtained on a second date subsequent to the first date; (c) automatically determine a registration field (e.g., a full 3D registration field; e.g., a pointwise registration) using landmarks automatically identified within the first and second 3D anatomical images (e.g., identified regions representing one or more of a cervical spine; thoracic spine; lumbar spine; left and right hip bones, sacrum and coccyx; left side ribs and left scapula; right side ribs and right scapula; left femur; right femur; skull, brain and mandible), and using, by the processor, the determined registration field to align the first and second PSMA-PET images [e.g., either before or after segmentation of the CT and/or PSMA-PET images to identify boundaries of organs and/or bones, and either before or after automatic hotspot (e.g., lesion) detection from the PSMA-PET images]; and (d) use the thusly aligned first and second PSMA-PET images to automatically detect (e.g., staging and/or quantifying) a change in (e.g., a progression of or remission of) the disease from the first date to the second date [e.g., automatically identifying, and/or identifying as such (e.g., tagging, labelling), one or both of (i) and (ii) as follows: (i) a change in the number of lesions {e.g., one or more new lesions (e.g., organ-specific lesions), or an elimination of one or more lesions (e.g., organ-specific)}, and (ii) a change in tumor size {e.g., an increase of tumor size (PSMA-VOL increase/decrease), e.g., total tumor size, or a decrease of tumor size (PSMA-VOL decrease)} {e.g., a change in volume of each of one or more specific lesions, or a change in overall volume of a specific type of lesions (e.g., organ-specific tumor), or a change in total volume of identified lesions}.

In certain embodiments, the system has one or more features and/or the instructions cause the processor to perform one or more steps articulated herein (e.g., in paragraphs above, for example at paragraphs [0057]-[0060]).

In another aspect, the invention is directed to a system of quantifying and reporting disease (e.g., tumor) burden for a patient having and/or at risk for cancer, the method comprising: a processor of a computing device; and memory having instructions stored thereon, wherein the instructions, when executed by the processor, cause the processor to: (a) obtain a medical image of the patient; (b) detect one or more (e.g., a plurality of) hotspots within the medical image, each hotspot corresponding to (e.g., being or comprising) a particular 3D volume [e.g., a 3D hotspot volume; e.g., wherein voxels of the 3D hotspot volume have an elevated intensity (e.g., and/or are otherwise indicative or increased radiopharmaceutical uptake) relative to their surroundings] within the medical image and representing a potential underlying physical lesion within the subject; (c) for each particular lesion class of a plurality of lesion classes representing specific tissue regions and/or lesion sub-types: identify a corresponding subset of the one or more hotspot(s) as belonging to the particular lesion class (e.g., based on a determination made, by the processor, that the hotspot represents an underlying physical lesion located within a particular tissue region and/or belonging to a particular lesion sub-type that the particular lesion class represents); and determine values of one or more patient index(indices) that quantify disease (e.g., tumor) burden within and/or associated with the particular lesion class based on the corresponding subset of hotspots; and (d) cause display of a graphical representation of the patient index values computed for each of the plurality of lesion classes (e.g., a summary table listing each lesion class and, for each lesion class, the computed patient index values), thereby providing a user with a graphical report summarizing tumor burden, within specific tissue regions and/or, associated with specific lesion sub-types.

In certain embodiments, the system has one or more features and/or the instructions cause the processor to perform one or more steps articulated herein (e.g., in paragraphs above, for example at paragraphs [0062]-[0067]).

In certain embodiments, the invention is directed to a system of characterizing and reporting individual lesions detected based on an imaging assessment of a patient having and/or at risk for cancer, the system comprising: a processor of a computing device; and memory having instructions stored thereon, wherein the instructions, when executed by the processor, cause the processor to: (a) obtain a medical image of the patient; (b) detect a set of one or more (e.g., a plurality of) hotspots within the medical image, each hotspot of the set corresponding to (e.g., being or comprising) a particular 3D volume [e.g., a 3D hotspot volume; e.g., wherein voxels of the 3D hotspot volume have an elevated intensity (e.g., and/or are otherwise indicative or increased radiopharmaceutical uptake) relative to their surroundings] within the medical image and representing a potential underlying physical lesion within the subject; (c) assign one or more lesion class labels to each of the one or more hotspots of the set, each lesion class label classes representing a specific tissue regions and/or lesion sub-types and identifying the hotspot as representing a potential lesion located within the specific tissue region and/or belonging to the lesion sub-type; (d) compute for each particular one of one or more individual hotspot quantification metrics, a value of the particular individual hotspot quantification metrics for each individual hotspot of the set; and (e) cause display of a graphical representation comprising, for each particular hotspot of at least a portion of the hotspots of the set, an identification of the particular hotspot (e.g., a row in a table, and, optionally, an alphanumeric identification, such as a number, identifying the particular hotspot) along with the one or more lesion class labels assigned to the particular hotspot and the values of the one or more individual hotspot quantification metrics computed for the particular hotspot [e.g., a summary table (e.g., a scrollable summary table) listing each individual hotspot as a row and the assigned lesion classes and hotspot quantification metrics column-wise].

In certain embodiments, the system has one or more features and/or the instructions cause the processor to perform one or more steps articulated herein (e.g., in paragraphs above, for example at paragraphs [0069]-[0072]).

In another aspect, the invention is directed to a system for quantifying and reporting disease (e.g., tumor) progression and/or risk over time for a patient having and/or at risk for cancer, the system comprising: a processor of a computing device; and memory having instructions stored thereon, wherein the instructions, when executed by the processor, cause the processor to: (a) obtain a plurality of medical images of the patient, each medical image representing a scan of the patient obtained at a particular time (e.g., a longitudinal dataset); (b) for each particular one of the plurality of medical images, detect a corresponding set of one or more (e.g., a plurality of) hotspots within the particular medical image, each hotspot corresponding to (e.g., being or comprising) a particular 3D volume [e.g., a 3D hotspot volume; e.g., wherein voxels of the 3D hotspot volume have an elevated intensity (e.g., and/or are otherwise indicative or increased radiopharmaceutical uptake) relative to their surroundings] within the medical image and representing a potential underlying physical lesion within the subject; (c) for each particular one of one or more (e.g., overall) patient index(indices) that measure (e.g., quantify) overall disease (e.g., tumor) burden within a patient at a particular time, determine a value of the particular (e.g., overall) patient index for each particular medical image of the plurality of medical images based on the corresponding set of hotspots detected for particular medical image, thereby determining, for each particular one of the one or more patient index(indices) a set of values tracking a change in disease burden as measured by to the particular patient index value over time; and (d) cause display of a graphical representation of the set of values for at least a portion (e.g., a particular one, a particular subset) of the one or more of the patient index values, thereby conveying a measure of disease progression over time for the patient.

In certain embodiments, the system has one or more features and/or the instructions cause the processor to perform one or more steps articulated herein (e.g., in paragraphs above, for example at paragraphs [0074]-[0076]).

In another aspect, the invention is directed to a system for automatically processing 3D images of a subject to determine values of one or more patient index(indices) that measure (e.g., overall) disease burden and/or risk for a subject, the system comprising: a processor of a computing device; and memory having instructions stored thereon, wherein the instructions, when executed by the processor, cause the processor to: (a) receive a 3D functional image of the subject obtained using a functional imaging modality; (b) segment a plurality of 3D hotspot volumes within the 3D functional image, each 3D hotspot volume corresponding to a local region of elevated intensity with respect to its surrounding and representing a potential cancerous lesion within the subject, thereby obtaining a set of 3D hotspot volumes; (c) compute for each particular one of one or more individual hotspot quantification metrics, a value of the particular individual hotspot quantification metric for each individual 3D hotspot volume of the set, wherein for a particular individual 3D hotspot volume, each hotspot quantification metric quantifies a property (e.g., intensity, volume, etc.) of the particular 3D hotspot volume and is (e.g., computed as) a particular function of intensities and/or a number of individual voxels within the particular 3D hotspot volume; and (d) determine the values of the one or more patient index(indices), wherein each of at least a portion of the patient indices is associated with one or more specific individual hotspot quantification metrics and is computed using the (e.g., same) particular function with intensities and/or number of voxels within a combined hotspot volume comprising (e.g., formed as a union of) at least a portion (e.g., substantially all; e.g., a particular subset) of the set of 3D hotspot volumes.

In certain embodiments, a particular patient index is an overall mean voxel intensity and is computed as an overall mean of voxel intensities located within the combined hotspot volume

In another aspect, the invention is directed to a system for automatically determining a prognosis for a subject with prostate cancer from one or more medical images of the subject [e.g., one or more PSMA PET images (PET images obtaining upon administering to the subject a PSMA targeting compound) and/or one or more anatomical (e.g., CT) images], the system comprising: a processor of a computing device; and memory having instructions stored thereon, wherein the instructions, when executed by the processor, cause the processor to: (a) receive and/or access the one or more images of the subject; (b) automatically determine a quantitative assessment of one or more prostate cancer lesions (e.g., metastatic prostate cancer lesions) from the one or more images [e.g., wherein the quantitative assessment comprises one or more members selected from the group consisting of (i) a molecular imaging TNM (miTNM) lesion type classification for local (T), pelvic-nodal (N), and/or extra-pelvic (M) disease (e.g., miT, miN, miMa (lymph), miMb (bone), miMc (other)); (ii) an indication of lesion location (e.g., prostate, iliac, pelvic bone, rib cage, etc.), (iii) a standard uptake value (SUV) (e.g., SUVmax, SUVpeak, SUVmean), (iv) a total lesion volume, (v) a change in lesion volume (e.g., individual lesion and/or total lesions), and (vi) a computed PSMA (aPSMA) score] (e.g., using one or more of the methods described herein); and (c) automatically determine from the quantitative assessment in (b), a prognosis for the subject, wherein the prognosis comprises one or more of the following for the subject: (I) an expected survival (e.g., months), (II) an expected time to disease progression, (III) an expected time to radiographic progression, (IV) a risk of concurrent (synchronous) metastases, and (V) a risk of future (metachronous) metastases.

In certain embodiments, the quantitative assessment of the one or more prostate cancer lesions determined in step (b) comprises one or more of the following: (A) a total tumor volume, (B) a change in tumor volume, (C) a total SUV, and (D) a PSMA score, and wherein the prognosis for the subject determined in step (c) comprises one or more of the following: (E) an expected survival (e.g., months), (F) a time to progression, and (G) a time to radiographic progression.

In certain embodiments, the quantitative assessment of the one or more prostate cancer lesions determined in step (b) comprises one or more characteristics of PSMA expression in prostate, and wherein the prognosis for the subject determined in step (c) comprises a risk of concurrent (synchronous) metastases, and/or a risk of future (metachronous) metastases.

In another aspect, the invention is directed to a system for automatically determining a response to a treatment for a subject with prostate cancer from a plurality of medical images of the subject [e.g., one or more PSMA PET images (PET images obtaining upon administering to the subject a PSMA targeting compound) and/or one or more anatomical (e.g., CT) images], the system comprising: a processor of a computing device; and memory having instructions stored thereon, wherein the instructions, when executed by the processor, cause the processor to: (a) receive and/or access, by a processor of a computing device, the plurality of images of the subject, wherein at least a first image of the plurality was obtained prior to an administration of the treatment and at least a second image of the plurality was obtained following administration of the treatment (e.g., after a period of time); (b) automatically determine a quantitative assessment of one or more prostate cancer lesions (e.g., metastatic prostate cancer lesions) from the images [e.g., wherein the quantitative assessment comprises one or more members selected from the group consisting of (i) a molecular imaging TNM (miTNM) lesion type classification for local (T), pelvic-nodal (N), and/or extra-pelvic (M) disease (e.g., miT, miN, miMa (lymph), miMb (bone), miMc (other)); (ii) an indication of lesion location (e.g., prostate, iliac, pelvic bone, rib cage, etc.), (iii) a standard physiological uptake value (SUV) (e.g., SUVmax, SUVpeak, SUVmean), (iv) a total lesion volume, (v) a change in lesion volume (e.g., individual lesion and/or total lesions), and (vi) a computed PSMA (aPSMA) score] (e.g., using one or more of the methods described herein) (e.g., wherein the quantitative assessment comprises Response Evaluation Criteria in PSMA-imaging (RECIP) criteria and/or PSMA PET Progression (PPP) criteria); and (c) automatically determine from the quantitative assessment in (b), whether the subject is responding to the treatment (e.g., a yes/no) and/or a degree to which the subject is responding to the treatment (e.g., a numerical value or classification).

In another aspect, the invention is directed to a system for automatically identifying whether a subject with prostate cancer (e.g., metastatic prostate cancer) is likely to benefit from a particular treatment for prostate cancer using a plurality of medical images of the subject [e.g., one or more PSMA PET images (PET images obtaining upon administering to the subject a PSMA targeting compound) and/or one or more anatomical (e.g., CT) images], the system comprising: a processor of a computing device; and memory having instructions stored thereon, wherein the instructions, when executed by the processor, cause the processor to: (a) receive and/or access the plurality of images of the subject; (b) automatically determine a quantitative assessment of one or more prostate cancer lesions (e.g., metastatic prostate cancer lesions) from the images [e.g., wherein the quantitative assessment comprises one or more members selected from the group consisting of (i) a molecular imaging TNM (miTNM) lesion type classification for local (T), pelvic-nodal (N), and/or extra-pelvic (M) disease (e.g., miT, miN, miMa (lymph), miMb (bone), miMc (other)); (ii) an indication of lesion location (e.g., prostate, iliac, pelvic bone, rib cage, etc.), (iii) a standard physiological uptake value (SUV) (e.g., SUVmax, SUVpeak, SUVmean), (iv) a total lesion volume, (v) a change in lesion volume (e.g., individual lesion and/or total lesions), and (vi) a computed PSMA (aPSMA) score] (e.g., using one or more of the methods described herein) (e.g., wherein the quantitative assessment comprises Response Evaluation Criteria in PSMA-imaging (RECIP) criteria and/or PSMA PET Progression (PPP) criteria); and (c) automatically determine, from the quantitative assessment in (b), whether the subject is likely to benefit from the particular treatment for prostate cancer [e.g., determining for the subject an eligibility score for one or more particular treatments and/or a class of treatments, e.g., a particular radioligand therapy, e.g., lutetium vipivotide tetraxetan (Pluvicto®)].

In another aspect, the invention is directed to a therapeutic agent for use in treatment (e.g., via multiple cycles of the therapeutic agent) of a subject having and/or at risk for a particular disease (e.g., prostate cancer (e.g., metastatic castration resistant prostate cancer)), the subject having been (i) administered a first cycle of the therapeutic agent and imaged (e.g., before and/or during and/or after the first cycle of the therapeutic agent) (ii) identified as a responder to the therapeutic agent using methods described herein, for example in paragraphs above, such as paragraphs [0003]-[0052]) (e.g., the subject having been identified/classified as a responder based on the values of the one or more risk indices determined using methods described herein, for example in paragraphs above, such as paragraphs [0003]-[0052])).

In another aspect, the invention is directed to a second (e.g., second-line) therapeutic agent for use in treatment of a subject having and/or at risk for a particular disease (e.g., prostate cancer (e.g., metastatic castration resistant prostate cancer)), the subject having been (i) administered a cycle of an initial, first, therapeutic agent and imaged (e.g., before and/or during and/or after the cycle of the first therapeutic agent) and (ii) identified as a non-responder to the first therapeutic agent using methods described herein, for example in paragraphs above, such as paragraphs [0003]-[0052]) (e.g., the subject having been identified/classified as a non-responder based on the values of the one or more risk indices determined using methods described herein, for example in paragraphs above, such as paragraphs [0003]-[0052]) (e.g., thereby moving the subject to a potentially more effective therapy).

Features of embodiments described with respect to one aspect of the invention may be applied with respect to another aspect of the invention.

### BRIEF DESCRIPTION OF THE DRAWING

The foregoing and other objects, aspects, features, and advantages of the present disclosure will become more apparent and better understood by referring to the following description taken in conjunction with the accompanying drawings, in which:

FIG. 1A is a set of corresponding slices of a CT image, a PET image, and a PET/CT fusion, obtained from a 3D PET/CT scan, according to an illustrative embodiment.

FIG. 1B is a set of two slices of a PET/CT composite image in which a PET image is overlaid on a CT scan, according to an illustrative embodiment.

FIG. 2 is a diagram illustrating an example process for segmenting an anatomical image and identifying anatomical boundaries in a co-aligned functional image, according to an illustrative embodiment.

FIG. 3 is a diagram illustrating an example process for segmenting and classifying hotspots, according to an illustrative embodiment.

FIG. 4A is a graphical user interface (GUI) showing a computer-generated report for a patient via image analysis and decision support tools of the present disclosure, according to an illustrative embodiment.

FIG. 4B is another screenshot of a computer-generated report that presents longitudinal data tracking disease burden and evolution over time, according to an illustrative embodiment.

FIG. 4C is a schematic showing an approach for computing lesion index values, according to an illustrative embodiment.

FIG. 5 is a block diagram showing an example process for tracking lesions and determining changes in hotspot quantification and/or patient index values.

FIG. 6A is a schematic illustrating evolution of hotspots identified at an initial, baseline scan and, subsequently, at a second, follow up, scan, according to an illustrative embodiment.

FIG. 6B is a schematic illustrating evolution of hotspots identified at an initial, baseline scan and, subsequently, at a second, follow up, scan, according to an illustrative embodiment.

FIG. 6C is a schematic illustrating evolution of hotspots identified at an initial, baseline scan and, subsequently, at a second, follow up, scan, according to an illustrative embodiment.

FIG. 7 is a block diagram of an example process for determining and using lesion correspondences to determine patient metric values and/or classifications, according to an illustrative embodiment.

FIG. 8 is a block diagram showing an example process for determining lesion correspondences, according to an illustrative embodiment.

FIG. 9A is an image showing an example registration performed using anatomical segmentation maps, according to an illustrative embodiment.

FIG. 9B is another image showing an example registration performed using anatomical segmentation maps, according to an illustrative embodiment.

FIG. 9C is another image showing an example registration performed using anatomical segmentation maps, according to an illustrative embodiment.

FIG. 10 is a set of three composite images (“First scan” shown twice for illustrative purposes) showing registration of a composite image obtained via a second scan to a composite image obtained via a first scan, according to an illustrative embodiment.

FIG. 11A is a schematic illustrating a registration between a second image obtained via a second scan and a first image obtained via a first scan, according to an illustrative embodiment.

FIG. 11B is a schematic illustrating a registration between a second image obtained via a second scan and a first image obtained via a first scan, according to an illustrative embodiment.

FIG. 12 is a set of three schematics showing three lesion correspondence metrics, according to an illustrative embodiment.

FIG. 13 is a block diagram of an exemplary cloud computing environment, used in certain embodiments.

FIG. 14 is a block diagram of an example computing device and an example mobile computing device used in certain embodiments.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### Certain Definitions

In order for the present disclosure to be more readily understood, certain terms are first defined below. Additional definitions for the following terms and other terms are set forth throughout the specification.

A, an: The articles “a” and “an” are used herein to refer to one or to more than one (i.e., at least one) of the grammatical object of the article. By way of example, “an element” means one element or more than one element. Thus, in this specification and the appended claims, the singular forms “a,” “an,” and “the” include plural references unless the context clearly dictates otherwise. Thus, for example, reference to a pharmaceutical composition comprising “an agent” includes reference to two or more agents.

About, approximately: As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

First, second, etc.: It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed or that the first element must precede the second element in some manner In addition, unless stated otherwise, a set of elements may comprise one or more elements.

Image: As used herein, an “image”—for example, a 3D image of subject, includes any visual representation, such as a photo, a video frame, streaming video, as well as any electronic, digital or mathematical analogue of a photo (e.g., a digital image), video frame, or streaming video, displayed or stored in memory (e.g., a digital image may, but need not be displayed for visual inspection). Any apparatus described herein, in certain embodiments, includes a display for displaying an image or any other result produced by the processor. Any method described herein, in certain embodiments, includes a step of displaying an image or any other result produced via the method. In certain embodiments, an image is a 3D image, conveying information that varies with position within a 3D volume. Such images may, for example, be represented digitally as a 3D matrix (e.g., a N×M×L matrix) with each voxel of a 3D image represented by an element of a 3D matrix. Other representations are also contemplated and included, for example, a 3D matrix may be reshaped as a vector (e.g., a 1×K size vector, where K is a total number of voxels) by stitching each row or column end to end. Examples of images include, for example, medical images, such as bone-scan images (also referred to as scintigraphy images), computed tomography (CT) images, magnetic resonance images (MRIs), optical images (e.g., bright-field microscopy images, fluorescence images, reflection or transmission images, etc.), positron emission tomography (PET) images, single-photon emission tomography (SPECT) images, ultrasound images, x-ray images, and the like. In certain embodiments, a medical image is or comprises a nuclear medicine image, produced from radiation emitted from within a subject being imaged. In certain embodiments, a medical image is or comprises an anatomical image (e.g., a 3D anatomical image) conveying information regarding location and extent of anatomical structures such as internal organs, bones, soft-tissue, and blood vessels, within a subject. Examples of anatomical images include, without limitation, x-ray images, CT images, MRIs, and ultrasound images. In certain embodiments, a medical image is or comprises a functional image (e.g., a 3D functional image) conveying information relating to physiological activities within specific organs and/or tissue, such as metabolism, blood flow, regional chemical composition, absorption, etc. Examples of functional images include, without limitation, nuclear medicine images, such as PET images, SPECT images, as well as other functional imaging modalities, such as functional MRI (fMRI), which measures small changes in blood flow for use in assessing brain activity.

Map: As used herein, the term “map” is understood to mean a visual display, or any data representation that may be interpreted for visual display, which contains spatially-correlated information. For example, a three-dimensional map of a given volume may include a dataset of values of a given quantity that varies in three spatial dimensions throughout the volume. A three-dimensional map may be displayed in two-dimensions (e.g., on a two-dimensional screen, or on a two-dimensional printout).

Segmentation map: As used herein, the term “segmentation map” refers to a computer representation that identifies one or more 2D or 3D regions determined by segmenting an image. In certain embodiments, a segmentation map distinguishably identifies multiple different (e.g., segmented) regions, allowing them to be individually and distinguishably accessed and operated upon and/or used for operating on, for example, one or more images.

3D, three-dimensional: As used herein, “3D” or “three-dimensional” with reference to an “image” means conveying information about three dimensions. A 3D image may be rendered as a dataset in three dimensions and/or may be displayed as a set of two-dimensional representations, or as a three-dimensional representation. In certain embodiments, a 3D image is represented as voxel (e.g., volumetric pixel) data.

Whole body: As used herein, the terms “full body” and “whole body” used (interchangeably) in the context of segmentation and other manners of identification of regions within an image of a subject refer to approaches that evaluate a majority (e.g., greater than 50%) of a graphical representation of a subject's body in a 3D anatomical image to identify target tissue regions of interest. In certain embodiments, full body and whole body segmentation refers to identification of target tissue regions within at least an entire torso of a subject. In certain embodiments, portions of limbs are also included, along with a head of the subject.

Radionuclide: As used herein, “radionuclide” refers to a moiety comprising a radioactive isotope of at least one element. Exemplary suitable radionuclides include but are not limited to those described herein. In some embodiments, a radionuclide is one used in positron emission tomography (PET). In some embodiments, a radionuclide is one used in single-photon emission computed tomography (SPECT). In some embodiments, a non-limiting list of radionuclides includes ^{99m}Tc, ₁₁₁In, ⁶⁴Cu, ⁶⁷Ga, ⁶⁸Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹²⁶I, ¹³¹I, ¹¹C, ¹³N, ¹⁵O, ¹⁸F, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, ⁸²Rb, ⁷⁵Br, ⁷⁶Br, ⁷⁷Br, ⁸⁰Br, ^{80m}Br, ⁸²Br, ⁸³Br, ²¹¹At and ¹⁹²Ir.

Radiopharmaceutical: As used herein, the term “radiopharmaceutical” refers to a compound comprising a radionuclide. In certain embodiments, radiopharmaceuticals are used for diagnostic and/or therapeutic purposes. In certain embodiments, radiopharmaceuticals include small molecules that are labeled with one or more radionuclide(s), antibodies that are labeled with one or more radionuclide(s), and antigen-binding portions of antibodies that are labeled with one or more radionuclide(s).

Machine learning module: Certain embodiments described herein make use of (e.g., include) software instructions that include one or more machine learning module(s), also referred to herein as artificial intelligence software. As used herein, the term “machine learning module” refers to a computer implemented process (e.g., function) that implements one or more specific machine learning algorithms in order to determine, for a given input (such as an image (e.g., a 2D image; e.g., a 3D image), dataset, and the like) one or more output values. For example, a machine learning module may receive as input a 3D image of a subject (e.g., a CT image; e.g., an MRI), and for each voxel of the image, determine a value that represents a likelihood that the voxel lies within a region of the 3D image that corresponds to a representation of a particular organ or tissue of the subject. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of a CNN module may be carried out via specialized hardware (e.g., via an application specific integrated circuit (ASIC)).

Subject: As used herein, a “subject” means a human or other mammal (e.g., rodent (mouse, rat, hamster), pig, cat, dog, horse, primate, rabbit, and the like).

Administering: As used herein, “administering” an agent means introducing a substance (e.g., an imaging agent) into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments.

Tissue: As used herein, the term “tissue” refers to bone (osseous tissue) as well as soft-tissue.

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

A. Nuclear Medicine Images

Nuclear medicine images may be obtained using a nuclear medicine imaging modality such as bone scan imaging (also referred to as scintigraphy), Positron Emission Tomography (PET) imaging, and Single-Photon Emission Tomography (SPECT) imaging.

In certain embodiments, nuclear medicine images are obtained using imaging agents comprising radiopharmaceuticals. Nuclear medicine images may be obtained following administration of a radiopharmaceutical to a patient (e.g., a human subject), and provide information regarding the distribution of the radiopharmaceutical within the patient.

Nuclear medicine imaging techniques detect radiation emitted from the radionuclides of radiopharmaceuticals to form an image. The distribution of a particular radiopharmaceutical within a patient may be influenced and/or dictated by biological mechanisms such as blood flow or perfusion, as well as by specific enzymatic or receptor binding interactions. Different radiopharmaceuticals may be designed to take advantage of different biological mechanisms and/or particular specific enzymatic or receptor binding interactions and thus, when administered to a patient, selectively concentrate within particular types of tissue and/or regions within the patient. Greater amounts of radiation are emitted from regions within the patient that have higher concentrations of radiopharmaceutical than other regions, such that these regions appear brighter in nuclear medicine images. Accordingly, intensity variations within a nuclear medicine image can be used to map the distribution of radiopharmaceutical within the patient. This mapped distribution of radiopharmaceutical within the patient can be used to, for example, infer the presence of cancerous tissue within various regions of the patient's body. In certain embodiments, intensities of voxels of a nuclear medicine image, for example a PET image, represent standard uptake values (SUVs) (e.g., having been calibrated for injected radiopharmaceutical dose and/or patient weight).

For example, upon administration to a patient, technetium 99m methylenediphosphonate (^{99m}Tc MDP) selectively accumulates within the skeletal region of the patient, in particular at sites with abnormal osteogenesis associated with malignant bone lesions. The selective concentration of radiopharmaceutical at these sites produces identifiable hotspots—localized regions of high intensity—in nuclear medicine images. Accordingly, presence of malignant bone lesions associated with metastatic prostate cancer can be inferred by identifying such hotspots within a whole-body scan of the patient. In certain embodiments, analyzing intensity variations in whole-body scans obtained following administration of ^{99m}Tc MDP to a patient, such as by detecting and evaluating features of hotspots, can be used to compute, risk indices that correlate with patient overall survival and other prognostic metrics indicative of disease state, progression, treatment efficacy, and the like. In certain embodiments, other radiopharmaceuticals can also be used in a similar fashion to ^{99m}Tc MDP.

In certain embodiments, the particular radiopharmaceutical used depends on the particular nuclear medicine imaging modality used. For example 18F sodium fluoride (NaF) also accumulates in bone lesions, similar to ^{99m}Tc MDP, but can be used with PET imaging. In certain embodiments, PET imaging may also utilize a radioactive form of the vitamin choline, which is readily absorbed by prostate cancer cells.

In certain embodiments, radiopharmaceuticals that selectively bind to particular proteins or receptors of interest—particularly those whose expression is increased in cancerous tissue may be used. Such proteins or receptors of interest include, but are not limited to tumor antigens, such as CEA, which is expressed in colorectal carcinomas, Her2/neu, which is expressed in multiple cancers, BRCA 1 and BRCA 2, expressed in breast and ovarian cancers; and TRP-1 and -2, expressed in melanoma.

For example, human prostate-specific membrane antigen (PSMA) is upregulated in prostate cancer, including metastatic disease. PSMA is expressed by virtually all prostate cancers and its expression is further increased in poorly differentiated, metastatic and hormone refractory carcinomas. Accordingly, radiopharmaceuticals that comprise PSMA binding agents (e.g., compounds that a high affinity to PSMA) labelled with one or more radionuclide(s) can be used to obtain nuclear medicine images of a patient from which the presence and/or state of prostate cancer within a variety of regions (e.g., including, but not limited to skeletal regions) of the patient can be assessed. In certain embodiments, nuclear medicine images obtained using PSMA binding agents are used to identify the presence of cancerous tissue within the prostate, when the disease is in a localized state. In certain embodiments, nuclear medicine images obtained using radiopharmaceuticals comprising PSMA binding agents are used to identify the presence of cancerous tissue within a variety of regions that include not only the prostate, but also other organs and tissue regions such as lungs, lymph nodes, and bones, as is relevant when the disease is metastatic.

In particular, upon administration to a patient, radionuclide labelled PSMA binding agents selectively accumulate within cancerous tissue, based on their affinity to PSMA. In a similar manner to that described above with regard to ^{99m}Tc MDP, the selective concentration of radionuclide labelled PSMA binding agents at particular sites within the patient produces detectable hotspots in nuclear medicine images. As PSMA binding agents concentrate within a variety of cancerous tissues and regions of the body expressing PSMA, localized cancer within a prostate of the patient and/or metastatic cancer in various regions of the patient's body can be detected, and evaluated. Various metrics that are indicative of and/or quantify severity (e.g., likely malignancy) of individual lesions, overall disease burden and risk for a patient, and the like, can be computed based on automated analysis of intensity variations in nuclear medicine images obtained following administration of a PSMA binding agent radiopharmaceutical to a patient. These disease burden and/or risk metrics may be used to stage disease and make assessments regarding patient overall survival and other prognostic metrics indicative of disease state, progression, treatment efficacy,

A variety of radionuclide labelled PSMA binding agents may be used as radiopharmaceutical imaging agents for nuclear medicine imaging to detect and evaluate prostate cancer. In certain embodiments, the particular radionuclide labelled PSMA binding agent that is used depends on factors such as the particular imaging modality (e.g., PET; e.g., SPECT) and the particular regions (e.g., organs) of the patient to be imaged. For example, certain radionuclide labelled PSMA binding agents are suited for PET imaging, while others are suited for SPECT imaging. For example, certain radionuclide labelled PSMA binding agents facilitate imaging a prostate of the patient, and are used primarily when the disease is localized, while others facilitate imaging organs and regions throughout the patient's body, and are useful for evaluating metastatic prostate cancer.

Several exemplary PSMA binding agents and radionuclide labelled versions thereof are described in further detail in Section H herein, as well as in U.S. Pat. Nos. 8,778,305, 8,211,401, and 8,962,799, and in U.S. Patent Publication No. US 2021/0032206 A1, the content of each of which are incorporated herein by reference in their entireties.

B. Image Segmentation in Nuclear Medicine Imaging

Nuclear medicine images are functional images. Functional images convey information relating to physiological activities within specific organs and/or tissue, such as metabolism, blood flow, regional chemical composition, and/or absorption. In certain embodiments, nuclear medicine images are acquired and/or analyzed in combination with anatomical images, such as computed tomography (CT) images. Anatomical images provide information regarding location and extent of anatomical structures such as internal organs, bones, soft-tissue, and blood vessels, within a subject. Examples of anatomical images include, without limitation, x-ray images, CT images, magnetic resonance images, and ultrasound images.

Accordingly, in certain embodiments, anatomical images can be analyzed together with nuclear medicine images in order to provide anatomical context for the functional information that they (nuclear medicine images) convey. For example, while nuclear medicine images, such as PET and SPECT convey a three-dimensional distribution of radiopharmaceutical within a subject, adding anatomical context from an anatomical imaging modality, such as CT imaging, allows one to determine the particular organs, soft-tissue regions, bones, etc. that radiopharmaceutical has accumulated in.

For example, a functional image may be aligned with an anatomical image so that locations within each image that correspond to a same physical location—and therefore correspond to each other—can be identified. For example, coordinates and/or pixels/voxels within a functional image and an anatomical image may be defined with respect to a common coordinate system, or a mapping (i.e., a functional relationship) between voxels within the anatomical image and voxels within the functional image established. In this manner, one or more voxels within an anatomical image and one or more voxels within a functional image that represent a same physical location or volume can be identified as corresponding to each other.

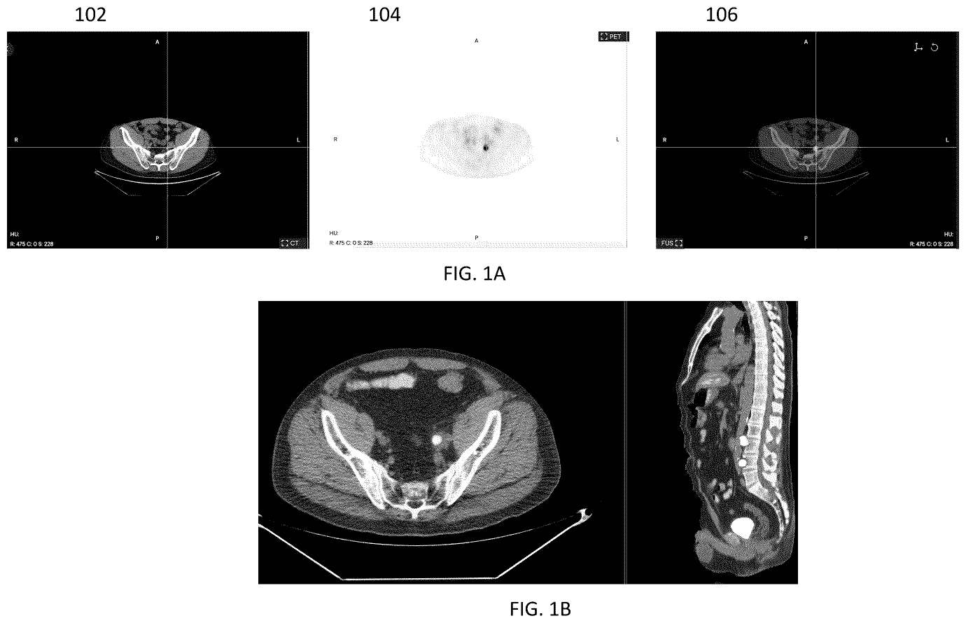

For example, FIG. 1 shows axial slices of a 3D CT image 102 and a 3D PET image 104, along with a fused image 106 in which the slice of the 3D CT image is displayed in grayscale and with the PET image is displayed as a semitransparent overlay. By virtue of the alignment between the CT and PET images, a location of a hotspot within the PET image, indicative of accumulated radiopharmaceutical and, accordingly a potential lesion, can be identified in the corresponding CT image, and viewed in anatomical context, for example, within a particular location in the pelvic region (e.g., within a prostate). FIG. 1B shows another PET/CT fusion, showing a transvers plane slice and a sagittal plane slice.

In certain embodiments, the aligned pair are a composite image, such as a PET/CT or SPECT/CT. In certain embodiments, an anatomical image (e.g., a 3D anatomical image, such as a CT image) and a functional image (e.g., a 3D functional image, such as a PET or SPECT image) are acquired using separate anatomical and functional imaging modalities, respectively. In certain embodiments, an anatomical image (e.g., a 3D anatomical image, such as a CT image) and a functional image (e.g., a 3D functional image, such as a PET or SPECT image) are acquired using a single multimodality imaging system. A functional image and an anatomical image may, for example, be acquired via two scans using a single multimodal imaging system—for example first performing a CT scan and then, second, performing a PET scan—during which a subject remains in a substantially fixed position.

In certain embodiments, 3D boundaries of particular tissue regions of interest can be accurately identified by analyzing 3D anatomical images. For example, automated segmentation of 3D anatomical images can be performed to segment 3D boundaries of regions such as particular organs, organ sub-regions and soft-tissue regions, as well as bone. In certain embodiments, organs such as a prostate, urinary bladder, liver, aorta (e.g., portions of an aorta, such as a thoracic aorta), a parotid gland, etc., are segmented. In certain embodiments, one or more particular bones are segmented. In certain embodiments, an overall skeleton is segmented.

In certain embodiments, automated segmentation of 3D anatomical images may be performed using one or more machine learning modules that are trained to receive a 3D anatomical image and/or a portion thereof, as input, and segment one or more particular regions of interest, producing a 3D segmentation map as output. For example as described in PCT publication WO/2020/144134, entitled “Systems and Methods for Platform Agnostic Whole Body Segmentation,” and published Jul. 16, 2020, the contents of which are incorporated herein by reference in their entirety, multiple machine learning modules implementing convolutional neural networks (CNNs) may be used to segment 3D anatomical images, such as CT images, of a whole body of a subject and thereby create a 3D segmentation map that identifies multiple target tissue regions across a subject's body.

In certain embodiments, for example to segment certain organs where functional images are believed to provide additional useful information that facilitate segmentation, a machine learning module may receive both an anatomical image and a functional image as input, for example as two different channels of input (e.g., analogous to multiple color channels in a color, RGB, image) and use these two inputs to determine an anatomical segmentation. This, multi-channel, approach is described in further detail in U.S. Patent Publication No. US 2021/0334974 A1, entitled “Systems and Methods for Deep-Learning-Based Segmentation of Composite Images,” and published Oct. 28, 2021, the contents of which is hereby incorporated by reference in its entirety.

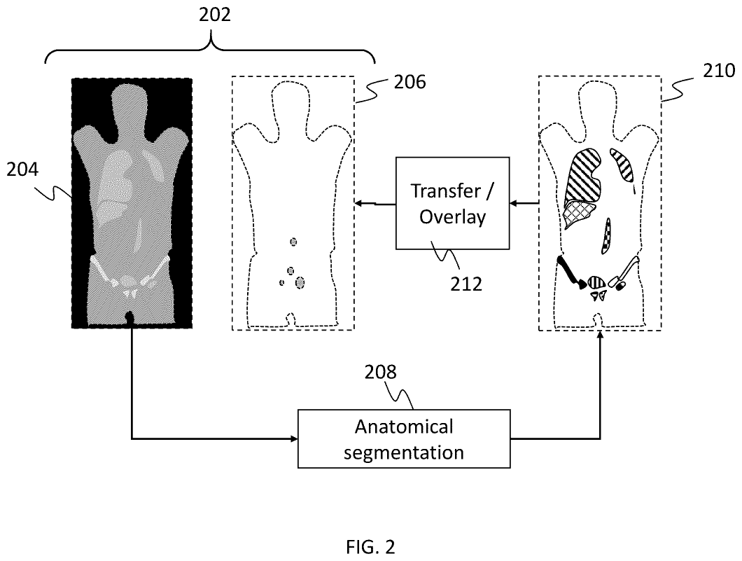

In certain embodiments, as illustrated FIG. 2, an anatomical image 204 (e.g., a 3D anatomical image, such as a CT image) and a functional image 206 (e.g., a 3D functional image, such as a PET or SPECT image) may be aligned with (e.g., co-registered to) each other, for example as in a composite image 202 such as a PET/CT image. Anatomical image 204 may be segmented 208 to create a segmentation map 210 (e.g., a 3D segmentation map) that distinguishably identifies one or more tissue regions and/or sub-regions of interest, such as one or more particular organs and/or bones. Segmentation map 210, having been created from anatomical image 204 is aligned with anatomical image 204, which, in turn, is aligned with functional image 206. Accordingly, boundaries of particular regions (e.g., segmentation masks), such as particular organs and/or bones, identified via segmentation map 210 can be transferred to and/or overlaid 212 upon functional image 206 to identify volumes within functional image 206 for purposes of classifying hotspots, and determining useful indices that serve as measures and/or predictions of cancer status, progression, and response to treatment. Segmentation maps and masks may also be displayed, for example as a graphical representation overlaid on a medical image to guide physicians and other medical practitioners.

C. Lesion Detection and Characterization

In certain embodiments, approaches described herein include techniques for detecting and characterizing lesions within a subject via (e.g., automated) analysis of medical images, such as nuclear medicine images. As described herein, in certain embodiments, hotspots are localized (e.g., contiguous) regions of high intensity, relative to their surroundings, within images, such as 3D functional images and may be indicative of a potential cancerous lesion present within a subject.

A variety of approaches may be used for detecting, segmenting, and classifying hotspots. In certain embodiments, hotspots are detected and segmented using analytical methods, such as filtering techniques including, but not limited to, a difference of Gaussians (DoG) filter and a Laplacian of Gaussians (LoG) filter. In certain embodiments, hotspots are segmented using a machine learning module that receives, as input, a 3D functional image, such as a PET image, and generates, as output a hotspot segmentation map (a “hotspot map”) that differentiates boundaries of identified hotspots from background. In certain embodiments, each segmented hotspot within a hotspot map is individually identifiable (e.g., individually labelled). In certain embodiments, a machine learning module used for segmenting hotspots may take as input, in addition to a 3D functional image, one or both of a 3D anatomical image (e.g., a CT image) and a 3D anatomical segmentation map. The 3D anatomical segmentation map may generated via automated segmentation (e.g., as described herein) of the 3D anatomical image.

In certain embodiments, segmented hotspots may be classified according to an anatomical region in which they are located. For example, in certain embodiments, locations of individual segmented hotspots within a hotspot map (representing and identifying segmented hotspots) may be compared with 3D boundaries of segmented tissue regions, such as various organs and bones, within a 3D anatomical segmentation map and labeled according to their location, e.g., based on proximity to and/or overlap with particular organs. In certain embodiments, a machine learning module may be used to classify hotspots. For example, in certain embodiments, a machine learning module may generate, as output, a hotspot map in which segmented hotspots are not only individually labeled and identifiable (e.g., distinguishable from each other), but are also labeled, for example, as corresponding to one of a bone, lymph, or prostate lesion. In certain embodiments, one or more machine learning modules may be combined with each other, as well as with analytical segmentation (e.g., thresholding) techniques to perform various tasks in parallel and in sequence to create a final labeled hotspot map.

Various approaches for performing detailed segmentation of 3D anatomical images and identification of hotspots representing lesions in 3D functional images, which may be used with various approaches described herein, are described in PCT publication WO/2020/144134, entitled “Systems and Methods for Platform Agnostic Whole Body Segmentation,” and published Jul. 16, 2020, U.S. Patent Publication No. US 2021/0334974 A1, entitled “Systems and Methods for Deep-Learning-Based Segmentation of Composite Images,” and published Oct. 28, 2021, and PCT publication WO/2022/008374, entitled “Systems and Methods for Artificial Intelligence-Based Image Analysis for Detection and Characterization of Lesions,” and published Jan. 13, 2022, the contents of each of which is incorporated herein in its entirety.

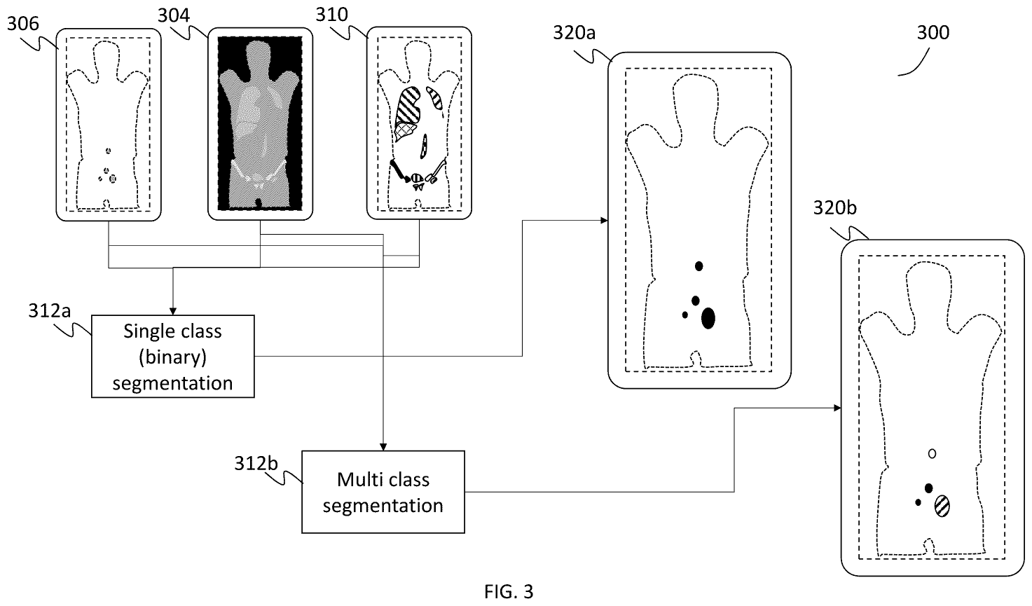

FIG. 3 shows an example process 300 for segmenting and classifying hotspots, based on an example approach described in further detail in PCT publication WO/2022/008374, entitled “Systems and Methods for Artificial Intelligence-Based Image Analysis for Detection and Characterization of Lesions,” and published Jan. 13, 2022. The approach illustrated in FIG. 3 uses two machine learning modules, each of which receives, as input, 3D functional image 306, 3D anatomical image 304, and 3D anatomical segmentation map 310. Machine learning module 312a is a binary classifier that generates a single-class hotspot map 320a, by labeling voxels as hotspot or background (not a hotspot). Machine learning module 312b performs multi-class segmentation, and generates multi-class hotspot map 320b, in which hotspots are both segmented and labeled as one of three classes—prostate, lymph, or bone. Among other things, classifying hotspots in this manner—via a machine learning module 312b (e.g., as opposed to directly comparing hotspot locations with segmented boundaries from segmentation map 310)—obviates a need to segment certain regions. For example, in certain embodiments, machine learning module 312b may classify hotspots as belonging to prostate, lymph, or bone, without a prostate region having be identified and segmented from 3D anatomical image 304 (e.g., in certain embodiments, 3D anatomical segmentation map 310 does not comprise a prostate region). In certain embodiments, hotspot maps 320a and 320b are merged, for example by transferring labels from multi-class hotspot map 320b to the hotspot segmentations identified in single-class hotspot map 320a (e.g., based on overlap). Without wishing to be bound to any particular theory, it is believed that this approach combines improved segmentation and detection of hotspots from single class machine learning module 312a with classification results from multi-class machine learning module 312b. In certain embodiments, hotspot regions identified via this final, merged, hotspot map are further refined, using an analytical technique such as an adaptive thresholding technique described in PCT publication WO/2022/008374, entitled “Systems and Methods for Artificial Intelligence-Based Image Analysis for Detection and Characterization of Lesions,” and published Jan. 13, 2022.

In certain embodiments, once detected and segmented, hotspots may be identified and assigned labels according to a particular anatomical (e.g., tissue) region in which they are located and/or a particular lesion sub-type that they are likely to represent. For example, in certain embodiments, hotspots may be assigned an anatomical location that identifies them as representing locations with a one of a set of tissue regions, such as the listed in Table 1, below. In certain embodiments, a list of tissue regions may include those in Table 1 as well as a gluteus maximus (e.g., left and right) and a gallbladder. In certain embodiments, hotspots are assigned to and/or labeled as belonging to a particular tissue region based on a machine learning classification and/or via comparison of their 3D hotspot volume's location and/or overlap with various tissue volumes identified via masks in an anatomical segmentation map. In certain embodiments, a prostate is not segmented. For example, as described above, in certain embodiments, machine learning module 312b may classify hotspots as belonging to prostate, lymph, or bone, without a prostate region having be identified and segmented from 3D anatomical image 304.

TABLE 1
Certain Tissue Regions (*Prostate may, optionally, be
segmented if present - may be absent if patient has,
e.g., undergone radical prostatectomy, or may not
segmented in any case, in certain embodiments)
Organs/Bones

Right and Right Lung
Left and Right Femur
Left and Right Hip Bone
Urinary bladder
Sacrum and coccyx
Liver
Spleen
Left and Right Kidney
Left Side and Right Side Ribs 1-12
Left and Right Scapula
Left and Right Clavicle
Cervical vertebrae
Thoracic vertebrae 1-12
Lumbar vertebrae 1-5
Sternum
Aorta, thoracic part
Aorta, abdominal part
Prostate*

In certain embodiments, additionally or alternatively, hotspots may be classified as belonging to one or more lesion sub-types. In certain embodiments, lesion sub-type classifications may be made by comparing hotspot locations with classes of anatomical regions. For example, in certain embodiments a miTNM classification scheme may be used, where hotspots are labeled as belonging to one of three classes—miT, miN, or miM—based on whether they represent lesions located within a prostate (miT), pelvic lymph node (miN), or a distant metastases (miM). In certain embodiments, a five class version of the miTNM scheme may be used, with distant metastases further divided into three sub classes—miMb for bone metastases, miMa for lymph metastases, and miMc for other soft tissue metastases.

For example, in certain embodiments, hotspots located within a prostate are labeled as belonging to class “T” or “miT”, e.g., representing local tumor. In certain embodiments, hotspots located outside a prostate, but within a pelvic region are labeled as class “N” or “miN”. In certain embodiments, for example as described in U.S. application Ser. No. 17/959,357, filed Oct. 4, 2022, entitled “Systems and Methods for Automated Identification and Classification of Lesions in Local Lymph and Distant Metastases,” published as U.S. 2023/0115732 A1 on Apr. 13, 2023, the content of which is incorporated herein by reference in its entirety, a pelvic atlas may be registered to identify boundaries of a pelvic region and/or various sub-regions therein, for purposes of identifying pelvic lymph node lesions. A pelvic atlas may, for example, include boundaries of a pelvic region and/or a planar reference (e.g., a plane passing through an aorta-bifurcation) which hotspot locations can be compared to (e.g., such that hotspots located outside the pelvic region and/or above the planar reference passing through an aorta bifurcation are labeled as “M” or “miM”—e.g., distant metastases). In certain embodiments, distant metastases may be classified as lymph (miMa), bone (miMb), or visceral (miMc) based on a comparison of hotspot locations with an anatomical segmentation map. For example, hotspots located within one or more bones (e.g., and outside a pelvic region) may be labeled as distant metastases, hotspots located within one or more segmented organs or a subset of organs (e.g., brain, lung, liver, spleen, kidneys) may be labeled as visceral (miMc) distant metastases, and remaining hotspots located outside a pelvic region labeled as distant lymph metastases (miMa).

Additionally or alternatively, in certain embodiments, hotspots may be assigned an miTNM class based on a determination that they are located within a particular anatomical region, for example based on a table such as Table 2, where each column corresponds to a particular miTNM label (first row indicating the particular miTNM class) and includes, in rows two and below, particular anatomical regions associated with each miTNM class. In certain embodiments, a hotspot can be assigned as being located within a particular tissue region listed in Table 2 based on a comparison of the hotspots location with an anatomical segmentation map, allowing for an automated miTNM class assignment.

TABLE 2
An Example List of Tissue Regions Corresponding to Five
Classes in a Lesion Anatomical Labeling Approach

| | | Pelvic lymph | | |
|---|---|---|---|---|
| Bone | Lymph nodes | nodes | Prostate | Visceral |
| Mb | Ma | N | T | Mc |
| Skull | Cervical | Template right | Prostate | Brain |
| Thorax | Supraclavicular | Template left | | Neck |
| Vertebrae | Axillary | Presacral | | Lung |
| lumbar | | | | |
| Vertebrae | Mediastinal | Other, pelvic | | Esophageal |
| thoracic | | | | |
| Pelvis | Hilar | | | Liver |
| Extremities | Mesenteric | | | Gallbladder |
| | Elbow | | | Spleen |
| | Popliteal | | | Pancreas |
| | Peri-/para-aortic | | | Adrenal |
| | Other, non- | | | Kidney |
| | pelvic | | | |
| | | | | Bladder |
| | | | | Skin |
| | | | | Muscle |
| | | | | Other |

In certain embodiments, hotspots may be further classified in terms of their anatomical location and/or lesion sub-type. For example, in certain embodiments, hotspots identified as located in pelvic lymph (miN) may be identified as belonging to a particular pelvic lymph node sub-region, such as one of a left/right internal iliac, a left or right external iliac, a left or right common iliac, a left or right obturator, a presacral region, or other pelvic region. In certain embodiments, distant lymph node metastases (miMa) may be classified as retroperitoneal (RP), supradiaphragmatic (SD), or other extrapelvic (OE). Approaches for regional (miN) and distant (miMa) lymph metastases classifications may include registration of pelvic atlas images and/or identification of various whole body landmarks, which are described in further detail in U.S. application Ser. No. 17/959,357, filed Oct. 4, 2022, entitled “Systems and Methods for Automated Identification and Classification of Lesions in Local Lymph and Distant Metastases,” published as U.S. 2023/0115732 A1 on Apr. 13, 2023, the content of which is incorporated herein by reference in its entirety.

D. Individual Hotspot Quantification Metrics

In certain embodiments, detected—e.g., identified and segmented—hotspots may be characterized via various individual hotspot quantification metrics. In particular, for a particular individual hotspot, individual hotspot quantification metrics can be used to quantify a measure of size (e.g., 3D volume) and/or intensity of the particular hotspot in a manner that is indicative of a size and/or level of radiopharmaceutical uptake within the (e.g., potential) underlying physical lesion that the particular hotspot represents. Accordingly, individual hotspot quantification metrics may convey, for example to a physician or radiologist, a likelihood that a hotspot appearing in an image represents a true underlying physical lesion and/or convey a likelihood or level of malignancy thereof (e.g., allowing to differentiate between benign and malignant lesions).

In certain embodiments, image segmentation, lesion detection, and characterization techniques as described herein are used to determine, for each of one or more medical images, a corresponding set of hotspots. As described herein, image segmentation techniques may be used to determine, for each hotspot detected in a particular image, a particular 3D volume—a 3D hotspot volume—representing and/or indicative of a volume (e.g., 3D location and extent) of a potential underlying physical lesion within the subject. Each 3D hotspot volume, in turn, comprises a set of image voxels, each having a particular intensity value.

Once determined, a set of 3D hotspot volumes may be used to compute one or more hotspot quantification metrics for each individual hotspot. Individual hotspot quantification metrics may be computed according to various methods and formulae described herein, for example below. In the description below, the variable L is used to refer to a set of hotspots detected with a particular image, with L={1, 2, . . . , l, . . . , N_{L}} representing a set of N_{L} (i.e., N_{L} being the number of hotspots) hotspots detected within an image and the variable l indexing the l^{th} hotspot. As described herein, each hotspot corresponds to a particular 3D hotspot volume within an image, with Rₗ denoting the 3D hotspot volume of the l^{th} hotspot.

Hotspot quantification metrics may be presented to a user via a graphical user interface (GUI) and/or a (e.g., automatically or semi-automatically) generated report. As described in further detail herein, individual hotspot quantification metrics may include hotspot intensity metrics and hotspot volume metrics (e.g., lesion volume) that quantify an intensity and size, respectively of a particular hotspot and/or underlying lesion it represents. Hotspot intensity and size may, in turn, be indicative of a level of radiopharmaceutical uptake within, and size of, respectively, an underlying physical lesion within the subject.

Hotspot Intensity Metrics

In certain embodiments, a hotspot quantification metric is or comprises an individual hotspot intensity metric that quantifies an intensity of an individual 3D hotspot volume. Hotspot intensity metrics may be computed based on individual voxel intensities within identified hotspot volumes. For example, for a particular hotspot, a value of a hotspot intensity metric may be computed as a function of at least a portion (e.g., a particular subset, e.g., all) of that hotspot's voxel intensities. Hotspot intensity metrics may include, without limitation, metrics such as a maximum hotspot intensity, a mean hotspot intensity, and peak hotspot intensity, and the like. As with voxel intensities in nuclear medicine images, in certain embodiments hotspot intensity metrics may represent (e.g., be in units of) SUV values.

In certain embodiments, a value of a particular hotspot intensity metric are computed, for a subject hotspot, based on (e.g., as a function of) that subject hotspot's voxel intensities alone, e.g., and not based on intensities of other image voxels outside the subject hotspot's 3D volume.

For example, a hotspot intensity metric may be a maximum hotspot intensity (e.g., SUV), or “SUV-max,” computed as a maximum voxel intensity (e.g., SUV or uptake) within a 3D hotspot volume. In certain embodiments, a maximum hotspot intensity may be computed according to equations (1a), (1b), or (1c), below

$${Q}_{max} ( l ) = {max}_{i ∈ {R}_{l}} ( {q}_{i} ) \qquad ( la )$$

$$SU {V}_{max} ( l ) = {max}_{i ∈ {R}_{l}} ( SU {V}_{i} ) \qquad ( 1 b )$$

$$SUV = max ( UptakeInVoxel ∈ lesion volume ) \qquad ( 1 c )$$

where, in equations (1a) and (1b) l represents a particular (e.g., l^{th}) hotspot, as described above, qᵢ is the intensity of voxel i and i∈Rₗ is the set of voxels within the particular 3D hotspot volume, Rₗ. In equation (1b), SUVᵢ indicates a particular unit—standard uptake value (SUV)—of voxel intensity, as described herein.

In certain embodiments, a hotspot intensity metric may be a mean hotspot intensity (e.g., SUV), or “SUV-mean,” and may be computed as a mean over all voxel intensities (e.g., SUV or uptake) within a 3D hotspot volume. In certain embodiments, a mean hotspot intensity may be computed according to equations (2a), (2b), or (2c) below.

$${Q}_{m e a n} ( l ) = {mean}_{i ∈ {R}_{l}} ( {q}_{i} ) = \frac{1}{{n}_{l}} {\sum }_{i ∈ {R}_{l}} {q}_{i} \qquad ( 2 a )$$

$$SU {V}_{m e a n} ( l ) = {mean}_{i ∈ {R}_{l}} ( {SUV}_{i} ) = \frac{1}{{n}_{l}} {\sum }_{i ∈ {R}_{l}} S U {V}_{i} \qquad ( 2 b )$$

$$SU {V}_{m e a n} = {\sum }_{i ∈ lesion volume} \frac{UptakeInVoxel}{{n}_{l}} \qquad ( 2 c )$$

where nₗ is the number of individual voxels within a particular 3D hotspot volume.

In certain embodiments, a hotspot intensity metric may be a peak hotspot intensity (e.g., SUV), or “SUV-peak,” and may be computed as a mean over intensities of the voxels (e.g., SUV or uptake) whose midpoints are located within a (e.g., pre-defined) particular distance (e.g., within 5 mm) of the midpoint of the hotspot voxel where the maximum intensity (e.g., SUV-max) is located within a hotspot, and, accordingly, may be computed according to equations (3a)-(3c) below.

$${Q}_{p e a k} ( l ) = \frac{1}{{n}_{l}} {\sum }_{{i :}_{dist ( {i}_{max} , i ) \leq d}} {q}_{i} \qquad ( 3 a )$$

$$SU {V}_{p e a k} ( l ) = \frac{1}{{n}_{l}} {\sum }_{{i :}_{dist ( {i}_{max} , i ) \leq d}} {SUV}_{i} \qquad ( 3 b )$$

$$SU {V}_{p e a k} = \frac{1}{{n}_{l}} {\sum }_{{{i :}_{dist ( {SUV}_{max} point , i )}}_{\leq 5 mm}} {UptakeInVoxel}_{i} \qquad ( 3 c )$$

where i: dist(iₘₐₓ, i)≤d is the set of (hotspot) voxels having a mid-point within a distance, d, from voxel iₘₐₓ, which is the maximum intensity voxel within the hotspot (e.g., Qₘₐₓ(l)=qᵢ₋ₘₐₓ.

Lesion Index Metrics

In certain embodiments, a hotspot intensity metric is individual lesion index value that maps an intensity of voxels within a particular 3D hotspot volume to a value on a standardized scale. Such lesion index values are described in further detail in PCT/EP2020/050132, filed Jan. 6, 2020 and PCT/EP2021/068337, filed Jul. 2, 2021, the content of each of which is hereby incorporated by reference in its entirety. Calculation of lesion index values may include calculation of reference intensity values within particular reference tissue regions, such as an aorta portion (also referred to as blood pool) and/or a liver.

For example, in one particular implementation, a first, blood-pool, reference intensity value is determined based on a measure of intensity (e.g., a mean SUV) within an aorta region and a second, liver, reference intensity value is determined based on a measure of intensity (e.g., a mean SUV) within a liver region. As described in further detail, for example in PCT/EP2021/068337, filed Jul. 2, 2021, the content of which is incorporated herein by reference in its entirety, calculation of reference intensities may include approaches such as identifying reference volumes (e.g., an aorta or portion thereof; e.g., a liver volume) within a functional image, such as a PET or SPECT image, eroding and/or dilating certain reference volumes, e.g., to avoid include voxels on the edge of a reference volume, and selecting subsets of reference voxel intensities, based on modeling approaches, e.g., to account for anomalous tissue features, such as cysts and lesions, within a liver. In certain embodiments, a third reference intensity value may be determined, either as a multiple (e.g., twice) of a liver reference intensity value, or based on an intensity of another reference tissue region, such as a parotid gland.

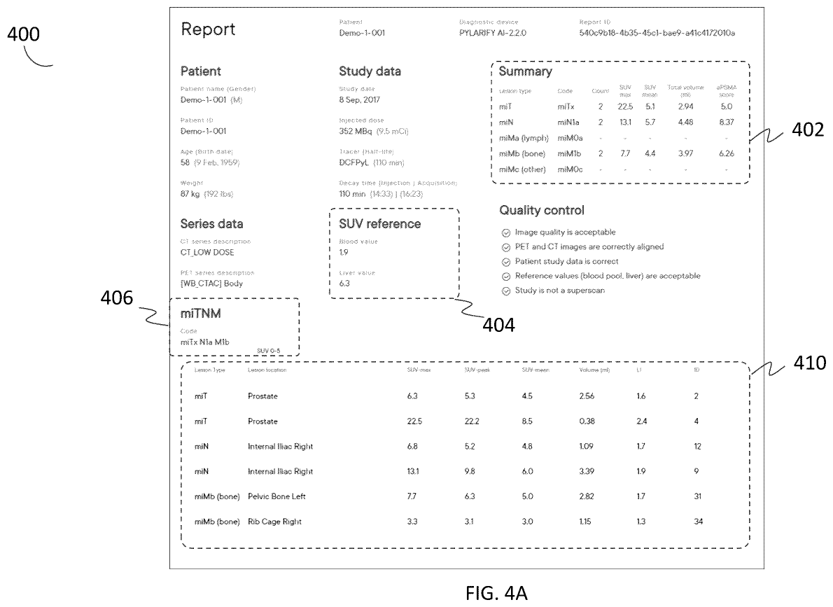

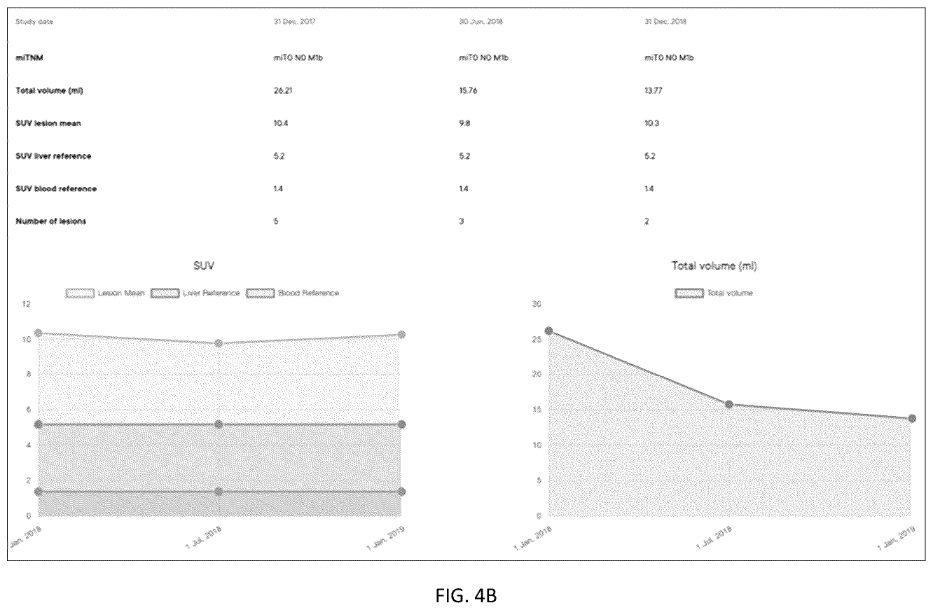

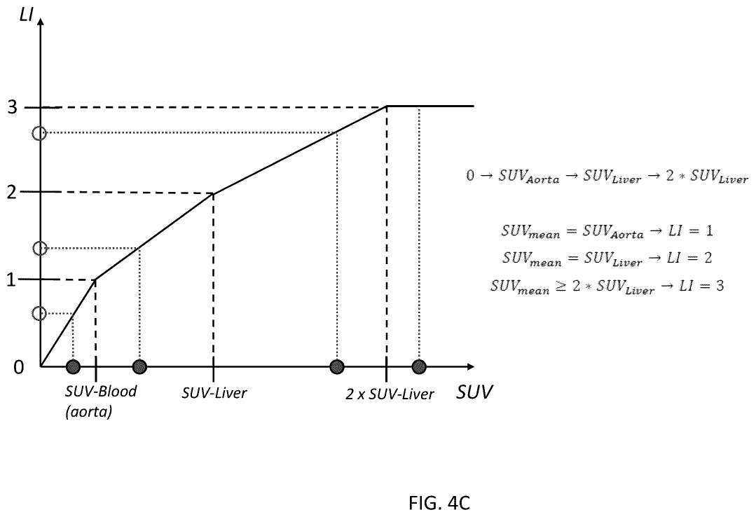

In certain embodiments, hotspot intensities may be compared with one or more reference intensity values to determine a lesion index as a value on a standardized scale, which facilitates comparison across different images. For example, FIG. 4C illustrates an approach for assigning hotspots a lesion index value ranging from 0 to 3. In the approach shown in FIG. 4C, a blood-pool (aorta) intensity value is assigned a lesion index of 1, a liver intensity value is assigned a lesion 2, and a value of twice the liver intensity is assigned a lesion index of 3. A lesion index for a particular hotspot can be determined by first computing a value of an initial hotspot intensity metric for the particular hotspot, such as a mean hotspot intensity (e.g., Qₘₑₐₙ(l) or SUVₘₑₐₙ) and comparing the value of the initial hotspot intensity metric with the reference intensity values. For example, the value of the initial hotspot intensity metric may fall within one of four ranges—[0, SUV_{blood}], (SUV_{blood}, SUVₗᵢᵥₑᵣ], (SUVₗᵢᵥₑᵣ, 2×SUVₗᵢᵥₑᵣ], and greater than 2×SUVₗᵢᵥₑᵣ (e.g., (2×SUVₗᵢᵥₑᵣ, ∞)). A lesion index value can then be computed for the particular hotspot based on (i) the value of the initial hotspot intensity metric and (ii) a linear interpolation according to the particular range in which the value of the initial hotspot intensity metric falls, as illustrated in FIG. 4C, where the filled and open dots on the horizontal (SUV) and vertical (LI) axes illustrate example values of initial hotspot intensity metrics and resultant lesion index values, respectively. In certain embodiments, if SUV references for either liver or aorta cannot be calculated, or if the aorta value is higher than the liver value, the Lesion Index will not be calculated and will be displayed as ‘-’.

A lesion index value according to the mapping scheme described above and illustrated in FIG. 4C may, for example, be computed as shown in equation (4), below.

$${Q}_{LI} ( l ) = \{ \begin{matrix}{f}_{1} ( S U {V}_{m e a n} ( l ) ) , & SU {V}_{m e a n} ( l ) \leq S U {V}_{aorta} \\ {f}_{2} ( S U {V}_{m e a n} ( l ) ) , & SU {V}_{aorta} \leq S U {V}_{m e a n} ( l ) \leq S U {V}_{liver} \\ {f}_{3} ( S U {V}_{m e a n} ( l ) ) , & SU {V}_{liver} \leq S U {V}_{m e a n} ( l ) \leq 2 \times S U {V}_{liver} \\ 3 , & 2 \times SU {V}_{liver} \leq S U {V}_{m e a n} ( l )\end{matrix} \qquad ( 4 )$$

where ƒ₁ ƒ₂ and ƒ₃ are linear interpolations between the respective spans in equation (4).

Hotspot/Lesion Volume

In certain embodiments, a hotspot quantification metric may be a volume metric, such as a lesion volume, Qᵥₒₗ, which provides a measure of size (e.g., volume) of an underlying physical lesion that a hotspot represents. A lesion volume may, in certain embodiments, computed as shown in equations (5a) and (5b), below.

$${Q}_{vol} ( l ) = {\sum }_{i ∈ {R}_{l}} {v}_{i} \qquad ( 5 a )$$

$${Q}_{vol} ( l ) = v \times {n}_{l} \qquad ( 5 b )$$

where in equation (5a), vᵢ is a volume of an i^{th} voxel, and equation (5b) assumes a uniform voxel volume, v, and as before nₗ is a number of voxels in a particular hotspot volume, l. In certain embodiments, a voxel volume is computed as v=δx×δy×δz, where δx, δy, and δz are grid spacing (e.g., in millimeters, mm) in x, y, and z. In certain embodiments, a lesion volume has units of milliliters (ml).

E. Aggregating Hotspot Metrics

In certain embodiments, systems and methods described herein compute patient index values that quantify disease burden and/or risk for a particular subject. Values of various patient indices may be computed using, for example as a function of, values of individual hotspot quantification measures. In particular, in certain embodiments, a particular patient index value aggregates values of multiple individual hotspot quantification metrics computed for an entire set of hotspots detected for the patient and/or for a particular subset of hotspots, for example associated with particular tissue regions and/or lesion sub-types. In certain embodiments, a particular patient index is associated with one or more specific individual hotspot quantification measures and is computed using the (e.g., multiple) values of the specific individual hotspot quantification metrics computed for each of at least a portion of the individual 3D hotspot volumes in the set.

Overall Patient Indices

For example, in certain embodiments, a particular patient index may be an overall patient index that aggregates values of one or more specific individual hotspot quantification measures computed across substantially an entire set of 3D hotspot volumes detected for a patient at a particular time point, to, for example, provide an overall measure of total disease burden for the subject at the particular time point.

In certain embodiments, a particular patient index may be associated with a single specific individual hotspot quantification measure and may be computed as a function of substantially all values of that specific individual hotspot quantification measure for the set of 3D hotspot volumes. Such patient indices may be viewed has having a functional form,

P_{p,m}=ƒ₍ₚ₎(Q_{(m),L}) (6)

where Q₍ₘ₎, denotes a particular individual hotspot quantification metric, such as Qₘₐₓ, Qₘₑₐₙ, Qₚₑₐₖ, Qᵥₒₗ, Q_{LI}, as described above, and Q_{(m),L} is the set of values of the specific individual hotspot quantification metric computed for each hotspot, l, in the set of hotspots L. That is, Q_{(m),L} is the set {Q₍ₘ₎(l=1), Q₍ₘ₎(l=2), . . . , Q₍ₘ₎(1−N_{L})}.

The function, ƒ₍ₚ₎, may be a variety of functions, which suitably aggregates (combines) the overall set of values of the particular specific individual hotspot quantification metric, Q₍ₘ₎. For example, the function ƒ₍ₚ₎ may be a sum, a mean, a median, a mode, a max, etc. Different particular functions may be used for ƒ₍ₚ₎, depending on the particular hotspot quantification metric, Q₍ₘ₎ that is being aggregated. Accordingly, various individual hotspot quantification measures (e.g., a mean intensity, a median intensity, a mode of intensities, a peak intensity, an individual lesion index, a volume) may be combined in a variety of manners, for example by taking an overall sum, mean, median, mode, etc., over substantially all values computed for the 3D hotspot volumes of the set.

For example, in certain embodiments, an overall patient index may be an overall intensity maximum, which is computed as a maximum over all individual hotspot maximum intensity values, as shown in equations (7a) or (7b), below

$${P}_{max} = max ( {Q}_{max , L} ) = {max}_{l ∈ L} {Q}_{max} ( l ) \qquad ( 7 a )$$

$${P}_{max} = max ( S U {V}_{max , L} ) = {max}_{l ∈ L} SU {V}_{max} ( l ) \qquad ( 7 b )$$

where Qₘₐₓ(l) may be computed according to equation (1a), above, in general, or according to equations (1b) or (1c) where image intensities represent SUV values, for example, as reflected in equation (7b).

In certain embodiments a particular patient index value may be computed as a combination of substantially all individual hotspot mean intensity values, for example as a sum of the mean intensity values, e.g., as shown in equations (8a) and (8b), below.

$${P}_{s u m} = {\sum }_{l ∈ L} {Q}_{m e a n} ( l ) \qquad ( 8 a )$$

$${P}_{s u m} = {\sum }_{l ∈ L} S U {V}_{m e a n} ( l ) \qquad ( 8 b )$$

In certain embodiments, an overall patient index is a total lesion volume, computed, for example, as a sum over all individual hotspot volumes, thereby providing a measure of total lesion volume. A total lesion volume may, for example, be computed as shown in equation (9a) and/or (9b), below,

$${P}_{V o l} = {\sum }_{l ∈ L} {Q}_{vol} ( l ) = {\sum }_{l ∈ L} {\sum }_{i ∈ {R}_{l}} {v}_{i} \qquad ( 9 a )$$

$${P}_{V o l} = {\sum }_{l ∈ L} {Q}_{vol} ( l ) = v {\sum }_{l ∈ L} {n}_{l} \qquad ( 9 b )$$

where (9b) assumes a uniform voxel size—i.e., each voxel has a same volume, vᵢ=v.

In certain embodiments, an overall patient index may be computed (e.g., directly) as a function of intensities, volumes, and/or number of voxels within the entire set of hotspots (e.g., as a function of all hotspot voxels within a union of all 3D hotspot volumes; e.g., not necessarily a function of individual hotspot quantification metrics). For example, in certain embodiments a patient index may be an overall mean value, and may be computed, for example, as shown in equations (10a) and (10b), below (i.e., by summing up intensities of all individual hotspot voxels for an entire set of hotspots, L, and dividing by a total number of hotspot voxels (for the entire set, L)):

$${P}_{m e a n} = \frac{{Σ}_{l ∈ L} {Σ}_{i ∈ {R}_{l}} {q}_{i}}{{Σ}_{l ∈ L} {n}_{l}} \qquad ( 10 a )$$

$${P}_{m e a n} = \frac{{Σ}_{l ∈ L} {Σ}_{i ∈ {R}_{l}} S U {V}_{i}}{{Σ}_{l ∈ L} {n}_{l}} \qquad ( 10 b )$$

In certain embodiments, a particular patient index may be computed using two or more specific individual hotspot quantification measures, e.g.,

P_{p,m}=ƒ₍ₚ₎Q_{(m1),L},Q_{(m2),L} . . . ) (11)

For example, an intensity-weighted measure of volume may be computed using both a measure of hotspot intensity and a measure of hotspot volume. For example, an intensity-weighted total volume may be computed at a patient level by computing, for each individual hotspot, a product of a lesion index computed for the individual hotspot and a volume of the hotspot. A sum over substantially all intensity-weighted volumes may then be computed to determine a total score according to, for example, the equation below, in which Q_{LI}(l) and Qᵥₒₗ(l) are the values of the individual lesion index and volume, respectively, for the i^{th} 3D hotspot volume.

$${P}_{ILV} = {\sum }_{l ∈ L} {Q}_{LI} ( l ) \times {Q}_{v o l} ( l ) \qquad ( 12 )$$

Other measures of intensity, for example as described above, may be used to weight a hotspot volume or compute version other metrics. In certain embodiments, additionally or alternatively, a patient index may be determined by multiplying a total lesion volume (e.g., as computed in equations (9a) or (9b)) by total SUV mean (e.g., as computed in equations (10a) or (10b)) to provide an assessment that also combines intensity with volume.

In certain embodiments, a patient index is or comprises a total lesion count, computed a total number of substantially all hotspots detected (e.g., N_{L}).

Region and Lesion Sub-Type Stratified Patient Indices

In certain embodiments, additionally or alternatively, multiple values of a particular patient index may be computed, each value associated with and computed for a particular subset of the 3D hotspot volumes (e.g., as opposed to the set L of substantially all hotspots).

In particular, in certain embodiments, 3D hotspot volumes within the set may be arranged in l assigned to one or more subsets according to, for example, particular tissue regions in which they are located or a sub-type based on a classification scheme, such as the miTNM classification. Approaches for grouping hotspots according to tissue regions and/or according to an anatomical classification such as miTNM are described in further detail in in PCT/EP2020/050132, filed Jan. 6, 2020 and PCT/EP2021/068337, filed Jul. 2, 2021, the content of each of which is hereby incorporated by reference in its entirety.

In this manner, values of patient indices as described herein may be computed for one or more particular tissue regions, such as a skeletal region, a prostate, or a lymph region. In certain embodiments, lymph regions may be further stratified in a finely grained fashion, for example using approaches as described PCT/EP22/77505, filed Oct. 4, 2022 (published as WO2023/057411 on Apr. 13, 2023), the content of which is hereby incorporated by reference in its entirety. Additionally or alternatively, in certain embodiments, each 3D hotspot volume may be assigned a particular miTNM sub-type and grouped into subsets according to the miTNM classification, and values of various patient indices may be computed for each miTNM classification.

For example, where hotspots are assigned a particular lesion sub-type according to the miTNM staging system, miTNM class-specific versions of the overall patient indices described above. For example, in certain embodiments, hotspots may be identified (e.g., automatically, based on their location) as local tumor (T), intrapelvic nodes (N), or distant metastases (M), and assigned a label such as miT, miN, and miM, respectively, to identify three subsets. In certain embodiments, distant metastases may be further subdivided accordingly to whether the lesion appears (e.g., as determined by hotspot location) in a distant lymph node region (a), a bone (b), or other site, such as another organ (c). Hotspots may thus be assigned one of five lesion (e.g., miTNM) classes (e.g., miT, miN, miMa, miMb, miMc). Accordingly, each hotspot may be assigned to a particular subset, S, such that, for example, values of a patient index P(S) may be computed for each subset, S, of hotspots within an image. For example, equations (13a-d), below, can be used to calculate patient index values for particular subsets of hotspots.

$${P}_{max} ( S ) = max ( {Q}_{max , S} ) = {max}_{l ∈ S} {Q}_{max} ( l ) \qquad ( 13 a )$$

$${P}_{m e a n} ( S ) = \frac{{\sum }_{l ∈ S} {\sum }_{i ∈ {R}_{l}} {q}_{i}}{{\sum }_{l ∈ S} {n}_{l}} \qquad ( 13 b )$$

$${p}_{Vol} ( S ) = {\sum }_{l ∈ S} {Q}_{vol} ( l ) = v {\sum }_{l ∈ S} {n}_{l} \qquad ( 13 c )$$

$${P}_{ILV} ( S ) = {\sum }_{l ∈ S} {Q}_{LI} ( l ) \times {Q}_{v o l} ( l ) \qquad ( 13 d )$$

where S denotes a particular subset of hotspots, such as local tumor (e.g., miT), intrapelvic nodes (e.g., labeled miN), distant metastases (e.g., labeled miM) or a particular type of distant metastases, such as a distant lymph node (e.g., labeled miMa), a bone (e.g., labeled miMb), or other site (e.g., labeled miMc). In each of equations (13a)-(13d), l∈S denotes the hotspots within subset S. Equation (13a) is analogous to equation (7a), with Q_{max,S} denoting the maximum hotspot intensity for hotspots within the subset S, and where Qₘₐₓ(l) may be computed according to equation (1a), above, in general, or according to equations (1b) or (1c) where image intensities represent SUV values. Equation (13b) is analogous to equation (10a), with qᵢ denoting the intensity (which may be in SUV units) of the i^{th} voxel and the combined hotspot volume over which the mean is taken is the union of all hotspot volumes within subset S. Equation (13c) is analogous to equation (9b), and gives an overall lesion volume for a particular subset, S. Equation (13d) is analogous to equation (12), and provides an overall intensity weighted lesion volume over a particular subset, S.

In certain embodiments, a lesion count may be computed a number of substantially all detected hotspots within a particular subset, S (e.g., Ns).

Scaled Patient Index Values

In certain embodiments, various patient index values may be scaled, for example accordingly to physical characteristics of a subject (e.g., weight, height, BMI, etc.) and/or volumes of tissue regions (e.g., a volume of a total skeletal region, a prostate volume, a total lymph volume, etc.) determined by analyzing (e.g., 3D anatomical images) images of the subject.

Reporting Patient Index Values

Turning to FIG. 4A, patient index values computed as described herein may be displayed (e.g., in a chart, graph, table, etc.) in a report (e.g., an automatically generated report), such as an electronic document or a portion of a graphical user interface, for example for review and validation/sign-off by a user.

Among other things, as shown in FIG. 4A, a generated report 400 as described herein may include a summary of patient index values 402 that quantify disease burden in the patient, for example grouping hotspot subsets according to a lesion type (e.g., an miTNM classification) and displaying, for each lesion type, one or more computed patient index values for that subtype. For example, summary portion 402 of report 400 displays patient index values for five subsets of hotspots, labeled miT, miN, miMa(lymph), miMb(bone), and miMc(other), based on the miTNM staging system. For each lesion sub-type, summary table 402 displays a number of detected hotspots belonging to that sub-type (e.g., within the particular subset), a maximum SUV (SUVₘₐₓ), a mean SUV (SUVmean), a total volume, and a quantity referred to as “aPSMA score”. For each lesion sub-type, S, values for SUVmax, SUVmean, Total volume, and aPSMA score may be computed as described above, for example, according to equations (13a), (13b), (13c), and (13d), respectively. In FIG. 4A, the term “aPSMA score” is used to reflect use of a PSMA binding agent, such as [18F]DCFPyL for imaging.

Summary table 402 in FIG. 4A also includes, for each lesion type, an alpha numeric code (e.g., miTx, miN1a, miM0a, miM1b, miM0c, displayed from top to bottom) that characterizes a severity, number, and location of lesions in the various regions, in accordance with the whole-body miTNM staging system described in Siefert et al., “Second Version of the Prostate Cancer Molecular Imaging Standardized Evaluation Framework Including Response Evaluation for Clinical Trials (PROMISE V2),” Eur Urol. 2023 May; 83(5):405-412. doi: 10.1016/j.eururo.2023.02.002. The notation miTx, for the miT (local tumor) sub-type uses “x” as a placeholder for various alpha numeric codes used in the miTNM system to indicate, for example, whether the local tumor is unifocal or multifocal, whether it is organ-confined or has invaded structures such as the seminal vesicle(s), other adjacent structures such as the external sphincter, rectum, bladder, levator muscles, pelvic wall, and whether it represents a local recurrence after radical prostatectomy. In certain embodiments, such finely-grained information may not be computed, for example due to particular imaging parameters and/or particular anatomical structures segmented. In certain embodiments, additional, finely-grained numeric (e.g., miT2, miT3, miT4) and alphanumeric (e.g., miT2u, miT2m, miT3a, miT3b, miT4, miTr) coding may be computed (e.g., automatically, based on automated anatomical segmentation) and displayed. In certain embodiments, such coding may be computed, but not displayed (e.g., intentionally) in a report such as 400 for sake of simplicity/readability of the report (e.g., to avoid overloading the physician or radiologist). Where level of detail in information, such as detailed miTNM (or other staging systems) coding information, displayed in a high-level report may be limited (e.g., intentionally), systems and methods described herein may include features for providing additional detail. For example, in providing a report such as report 400 via a graphical user interface, a user may be provided with the option to view additional coding information, for example by clicking (or tapping, e.g., in a touch screen device) on or hovering a mouse over portions of report 400. For example, a click or touch interaction may be used to expand summary table 402, allowing for a larger view where additional coding information can be presented, or a click on a particular code, such as “miTx” may be used to bring up (e.g., via a pop-up) additional information.

Generated reports, such as report 400, may also include information such as reference values (e.g., SUV uptake) 404 determined for various reference organs, such as a blood pool (e.g., computed from an aorta region or portion thereof) and a liver, which quantify physiological uptake within the patient, a disease stage code 406, such as an alphanumeric code based on the miTNM scheme, or other schemes. In certain embodiments, disease stage representation 406 includes an indication of the particular staging criteria used. For example, as shown in FIG. 4A, disease stage representation 406 includes the text “miTNM” to indicate use of miTNM staging criteria, along with the particular code determined via analysis of the particular scan(s) on which report 400 is based.

A report may include, additionally or alternatively, a hotspot table 410 that provides a list of each individual hotspot identified, with, for each hotspot, information such as a lesion sub-type, a lesion location (e.g., particular tissue volume in which the lesion is located), and values of various individual hotspot quantification metrics as described herein.

A report as shown in FIG. 4A may, accordingly, be generated from a single imaging session (e.g., a functional and anatomical image, such as a PET/CT or SPECT/CT image) and be used to provide a snapshot of a patient's disease at a particular time.

In certain embodiments, as described in further detail herein, multiple images, taken over time, may be used to track disease evolution over time. Such information may also be included in a report or portion thereof, for example as shown in FIG. 4B.

F. Lesion Tracking Across Medical Images

In certain embodiments, among other things, image analysis and decision support tools of the present disclosure provide systems and methods for tracking lesions and evaluating disease progression and/or treatment response in patients via analysis of nuclear medicine images. In particular, in certain embodiments, approaches described herein may be used to analyze longitudinal image data—i.e., a series of medical images (e.g., two or more images) collected over time.

Lesion tracking techniques described herein may be used in connection with a variety of medical image types and/or imaging modalities. For example, a medical image may be or comprise an anatomical image. Anatomical images convey anatomical information about structures/morphology within a body of a subject and are obtained using an anatomical imaging modality such as CT, MRI, ultrasound, etc.

While described herein in particular with respect to tracking lesions across a time-series of medical images, lesion tracking approaches of the present disclosure may be used, additionally or alternatively, to identify lesion correspondences between medical images (e.g., of a same subject) obtained using different imaging agents (e.g., different radiopharmaceuticals), dosages thereof, image reconstruction techniques, acquisition equipment, such as different cameras, combinations thereof, etc.

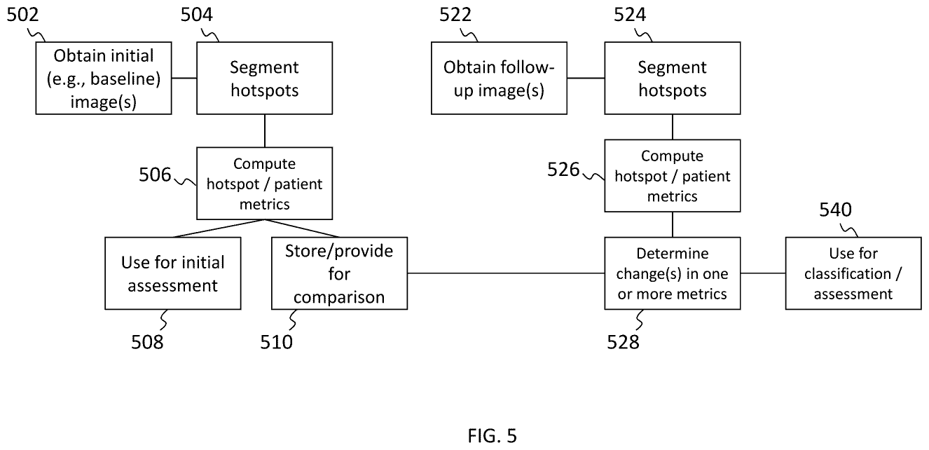

Turning FIG. 5, in certain embodiments, approaches herein may be used when a patient as had an initial, baseline, scan and, subsequently (e.g., at a later time) has a follow-up scan, e.g., to evaluate response to treatment and/or track his/her disease.

In certain embodiments, medical images analyzed via approaches described herein are or comprise nuclear medicine images, e.g., three-dimensional (3D) images, e.g., bone scan (scintigraphy) images, PET images, and/or SPECT images. In certain embodiments, the nuclear medicine image is supplemented (e.g., overlaid) with an anatomical image, e.g., a computed tomography (CT) image, X-ray, or MRI.

Following a patient's initial baseline scan, medical images, such are a PET/CT image, resulting from the scan are obtained 502 and analyzed to detect and segment hotspots 504 to identify image regions indicative of underlying cancerous lesions in a subject, for example as described herein (e.g., at Sections B and C).

Identified hotspots may be analyzed, for example, to compute values various individual hotspot quantification metrics and/or patient index metrics 506 as described herein. As described herein, hotspot quantification metrics may, for example, include intensity measures (e.g., peak, mean, median, etc., intensities within a particular hotspot), measures of size (e.g., hotspot volume), and lesion index values that combine both size and intensity, e.g., to give an overall severity of a particular underlying lesion. In certain embodiments, intensities of one or more reference organs, such as liver, aorta, parotid gland, may be used scale hotspot intensities, allowing for calculation of lesion index values on a standardized scale.

Individual hotspot quantification metrics may be combined/aggregated to provide an overall risk/disease severity picture for a patient overall and/or for particular anatomical regions (e.g., prostate, skeletal burden, lymph) and/or tumor classifications (e.g., various classes of lesions according to an miTNM classification or other scheme). For example, volumes of hotspots may be summed and/or otherwise aggregated over an entire patient (e.g., or a selected region) to compute a total lesion volume for a particular patient.

Values of hotspot quantification metrics and/or patient level risk metrics (patient indices) may be used, for example to provide an initial assessment for a patient, and/or may be stored and/or provided for further processing.

Turning again to FIG. 5, after a period of time (e.g., following a period of treatment), one or more follow-up images (time 2 images) are obtained 522, hotspots are identified 524, and quantification/risk metrics are computed 526 as discussed above. Changes in one or more metrics between the initial images and the time 2 images are computed. For example, (i) a change in the number of (automatically and/or semi-automatically) identified lesions may be identified and/or (ii) a change in an overall volume of (automatically and/or semi-automatically) identified lesions (e.g., a change in the sum of the volumes of each identified lesion) may be computed, and/or (iii) a change in PSMA (e.g., lesion index) weighted total volume (e.g., a sum of the products of lesion index and lesion volume for all lesions in a region of interest). Other metrics indicative of a change may also or alternatively be automatically determined. Similarly, further follow-up images can be obtained and analyzed thusly at later time points, e.g., time 3, time 4, etc. This longitudinal data set for lesion tracking may be used by a medical provider, for example, to determine effectiveness of a treatment.

For example, in certain embodiments, hotspot maps are retained with patient records and each follow-up map is compared to a baseline map (or a previous follow-up map) to identify corresponding (same) lesions, e.g., to identify which lesions are new, and/or to create per-lesion longitudinal data, allowing tracking, for each lesion, a volume, intensity, lesion index score, or other parameters. Thus, the methods described herein provide semi-automated and/or automated analysis of medical image data taken over time to produce a longitudinal dataset that provides a picture of how a patient's risk and/or disease evolves over time during surveillance and/or in response to treatment.

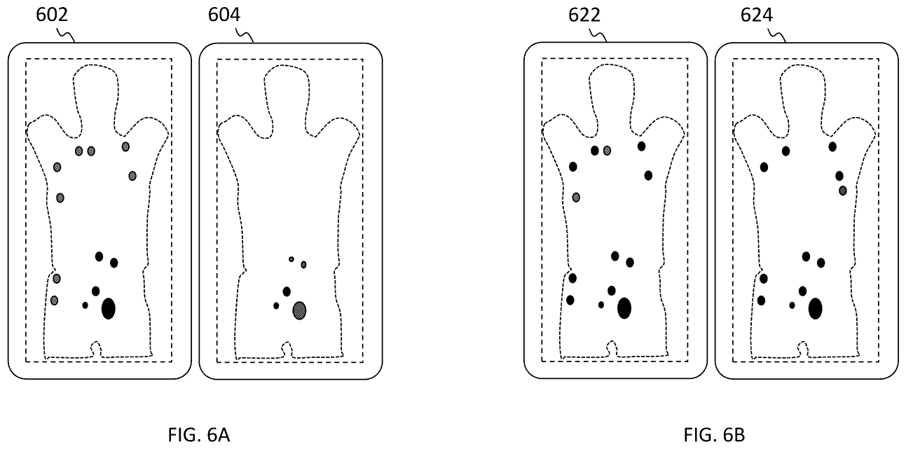

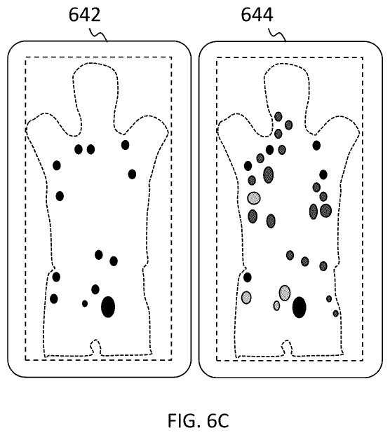

In certain embodiments, the methods described herein provide for computation of metrics that can be used to classify patient disease for treatment/decision making purposes and/or to stratify groups for clinical trial data collection and analysis. For example, in certain embodiments, changes in one or more metrics can be used to classify a patient as belonging to one of three categories—(i) response/partial response, characterized by a PSMA-Volume decline of greater than or equal to 30% and a decrease in number of lesions, shown in FIG. 6A; stable disease, characterized by a PSMA-volume decline of greater than 30%, but with appearance of new lesions (FIG. 6B) and (ii) progressive disease, characterized by an increase in PSMA-volume of 20% or more, and the appearance of one or more new lesions, e.g., as per RECIP classifications (FIG. 6C).

Registering Multiple Medical Images

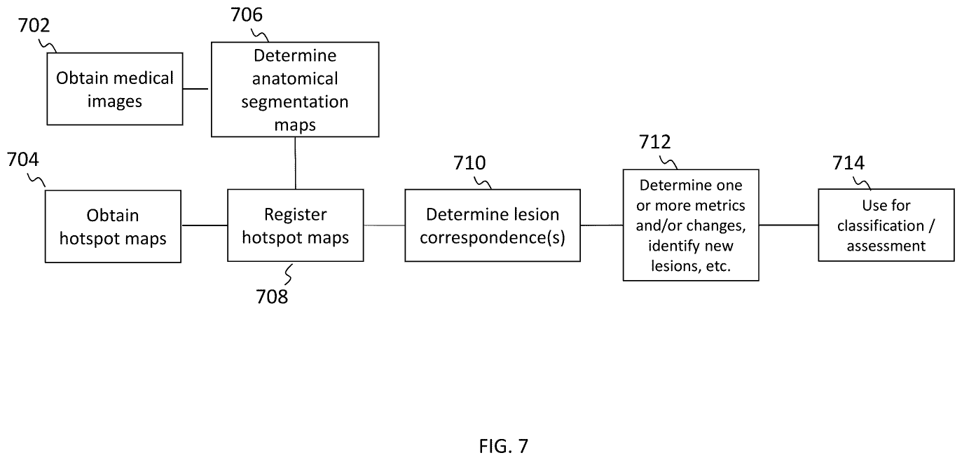

Turning to FIG. 7, in certain embodiments, two or more different medical images may be obtained 702, for example from a same subject at different time pointes (e.g., a time series). Each particular medical image may have, associated with it, a particular hotspot map that identifies one or more hotspots within the particular medical image. In certain embodiments, medical images and associated hotspot maps may be analyzed to identify corresponding hotspots in two or more medical images that are determined to represent a same underlying lesion. In this manner, presence (e.g., appearance and/or disappearance) and/or features, such as size/volume, radiopharmaceutical uptake, etc., of lesions may be compared between multiple different medical images.

In certain embodiments, a plurality of medical images may be or comprise a time series of medical images obtained for a same particular subject, each medical image, for example, having been obtained at a different time. Additionally or alternatively, a plurality of medical images may comprise medical images obtained using different imaging agents (e.g., different radiopharmaceuticals), dosages thereof, image reconstruction techniques, acquisition equipment, such as different cameras, combinations thereof, etc.

In certain embodiments, a plurality of hotspot maps may be obtained 704. Each hotspot map is associated with a particular medical image and identifies one or more hotspots therein. Hotspots are regions of interest (ROIs) identified within a particular medical image and/or sub-image thereof (e.g., in the case of a composite image) as representing potential underlying physical lesions within a subject. A hotspot map may identify hotspot volumes (e.g., 3D volumes), for example having been determined via segmentation of a 3D image.

In certain embodiments, hotspots are identified and/or segmented within a 3D functional image, for example as localized regions of elevated intensity.

In certain embodiments, hotspot maps may be generated via manual and/or automated detection and/or segmentation, or combinations thereof. Manual and/or semi-automated approaches may comprise receipt of a user input, for example via an image analysis graphical user interface (GUI). A user may review a rendering of one or more medical images and/or sub-images thereof, with or without various computer-generated annotations, such as organ segmentations displayed in combination, and perform operations such as selecting regions to include and/or exclude from a hotspot map. In certain embodiments, automated hotspot identification and segmentation is performed prior to a user review, to generate a preliminary hotspot map, which is then reviewed by a user, e.g., to generate a final hotspot map.

In certain embodiments, hotspots are classified (e.g., assigned labels) as belonging to particular anatomical regions (e.g., bone, lymph, pelvic, prostate, visceral (e.g., soft-tissue organs (other than prostate, lymph)—e.g., liver, kidney, spleen, lung, and brain)) and/or lesion categories, such as those of the miTNM classification scheme.

In certain embodiments, each medical image is segmented to identify a set of organ regions therein and create a corresponding anatomical segmentation map 706. An anatomical segmentation map identifies, within a particular medical image, a set of organ regions, each member of the set corresponding to a particular organ, including various soft-tissue and/or bone regions. As described herein, anatomical segmentation may be performed using a machine learning module. A machine learning module may receive an anatomical image, as input, and analyze the anatomical image to generate an anatomical segmentation map.

In certain embodiments, anatomical segmentation maps determined from each medical image may be used to perform image registration. In particular, at least a portion of the set of identified organ regions (e.g., comprising regions corresponding to one or more of a cervical spine; thoracic spine; lumbar spine; left and right hip bones, sacrum and coccyx; left side ribs and left scapula; right side ribs and right scapula; left femur; right femur; skull, brain and mandible) may be used to determine one or more registration fields that co-register two or more anatomical segmentation maps. Once determined, the one or more registration fields can be used to co-register medical images from which the anatomical segmentation maps were determined and/or their corresponding hotspot maps 708.

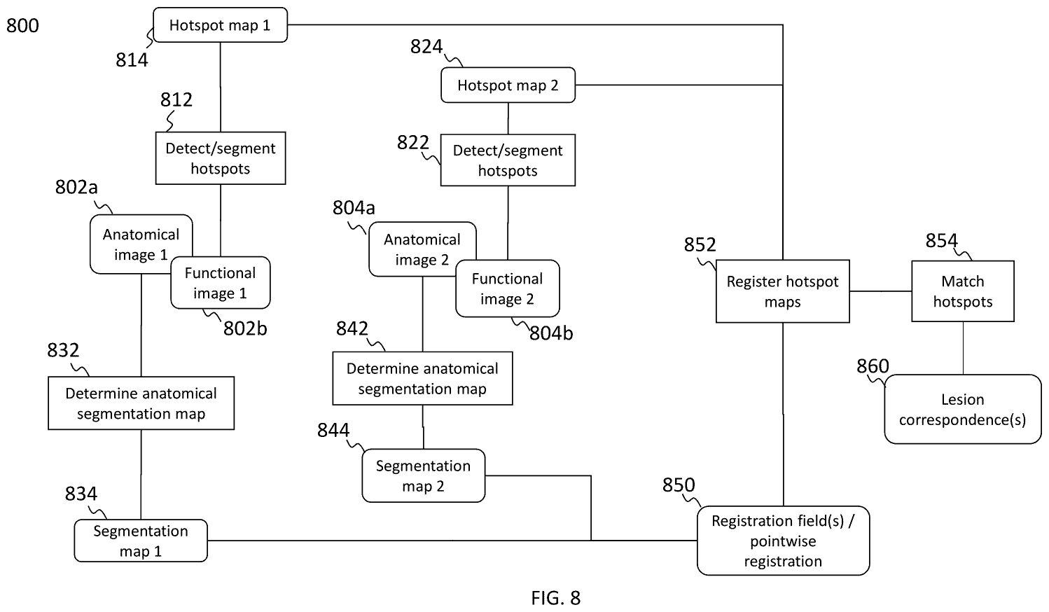

For example, turning to FIG. 8, this approach may be used to co-register a first and a second medical image and/or their corresponding hotspot maps. In process, 800 first and second medical image are composite images, each comprising an anatomical and functional image pair (802a/802b and 804a/804b).

First hotspot map 814 identifies a first set of hotspots within first medical image and may be and/or have been creating by detecting and/or segmenting hotspots 812 within first functional image 802b. Second hotspot map 824 identifies a second set of hotspots within second medical image and may be and/or have been creating by detecting and/or segmenting hotspots 822 within second functional image 804b.

First anatomical image 802a may be segmented 832, e.g., using a machine learning module—an anatomical segmentation module—to determine first anatomical segmentation map 834 that identifies, within first medical image (i.e., within first anatomical image and/or first functional image) a set of one or more organ regions. Second anatomical image 804a may be segmented 842, e.g., using the anatomical segmentation module—to determine a second anatomical segmentation map 844 that identifies, within second medical image (i.e., within second anatomical image and/or second functional image) the set of one or more organ regions.

Full Field Image Registration

In certain embodiments, first 834 and second 844 anatomical segmentation maps are used to determine one or more registration fields. Registration fields may be computed based on (e.g., to perform) an affine transformation. For example, in certain embodiments, one or more particular subsets of the set of identified organ regions are used as landmarks to register the first and second anatomical segmentation maps. In particular, each particular subset of identified organ regions may be used to determine a corresponding registration field that brings the particular subset within the first anatomical segmentation map into alignment with the same particular subset within the second anatomical segmentation map. This process may be performed for multiple subsets of identified organ regions to determine a plurality of registration fields 850, which can then be combined to create a final overall registration field used to perform a final image registration.

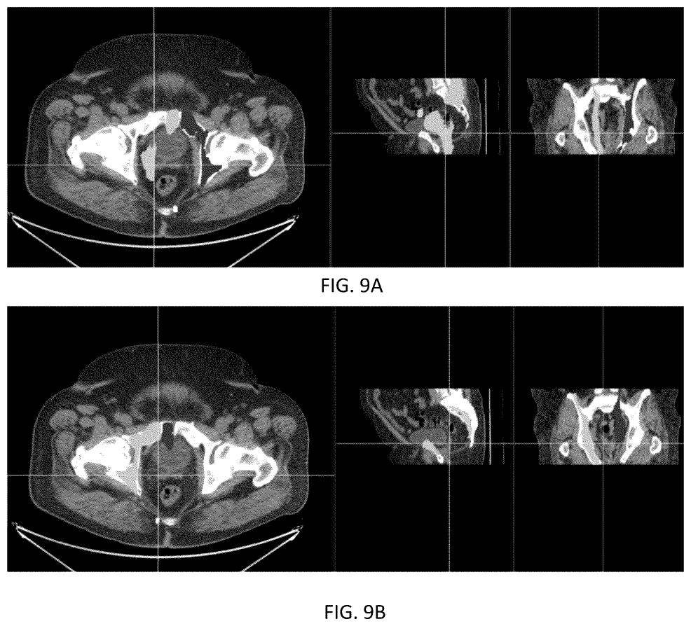

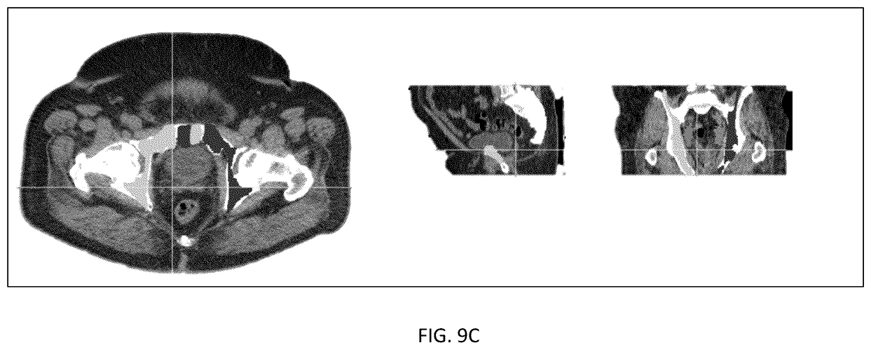

For example, each subset may comprise organ regions corresponding to locations within a particular anatomical region or portion of a subject's body. For example, as shown in FIGS. 9A and 9B, a first, left pelvic region, registration field may be determined using a subset of organ regions corresponding to pelvic bones on a left side of a subject (FIG. 9A), and a second, right pelvic region, registration field determined using a subset of organ regions corresponding to pelvic bones on a right side of the subject (FIG. 9B). As shown in FIG. 9C, these two (left and right pelvic region) registration fields may be combined, for example via a distance-weighted voxel-by-voxel average whereby each voxel of a final registration field is computed as a weighted average of the values for that voxel in the left and right pelvic region registration fields. For each voxel, the weights for the left and right voxel values used in the average may be determined based on a distance from that voxel to the identifications of the left and right pelvic bones, respectively. An example of this registration approach is described in further detail in PCT/EP22/77505, filed Oct. 4, 2022 (published as WO2023/057411 on Apr. 13, 2023) with respect to portions of images located about a pelvic region. This approach may be extended to a plurality of organ region subsets, throughout a subject's body (e.g., each organ subset associated with a particular portion of the body, such as a head, neck, chest, abdomen, pelvic region, left side, right side, front, back, etc. and combinations (e.g., left pelvic region, right pelvic region, left front chest, right front chest, etc.) thereof), so as to determine a plurality of local registration fields, each using a particular organ region subset as landmarks, which are then combined (e.g., via a distance-weighted average) to produce a final overall registration field.

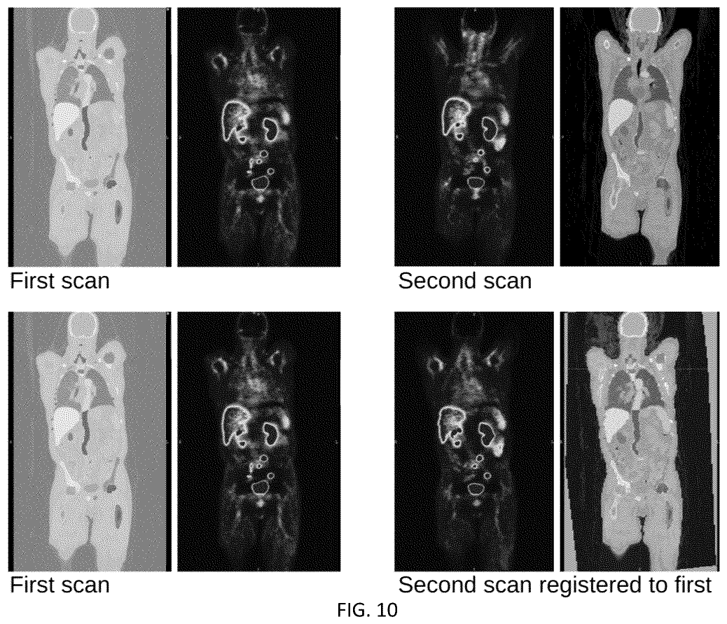

As shown in FIG. 10, this approach may be used to perform accurate full-body image registration. For example, FIG. 10 shows a first PET/CT composite image obtained via a first scan and a second PET/CT composite image, as initially obtained via a second scan (top row). Each CT scan shows identified organ regions of an anatomical segmentation map overlaid (colorized portions). The bottom row of FIG. 10 shows the first PET/CT image again, along with a transformed version of the second PET/CT image, which is now registered to the first image via a weighted piecewise affine registration approach as described herein.

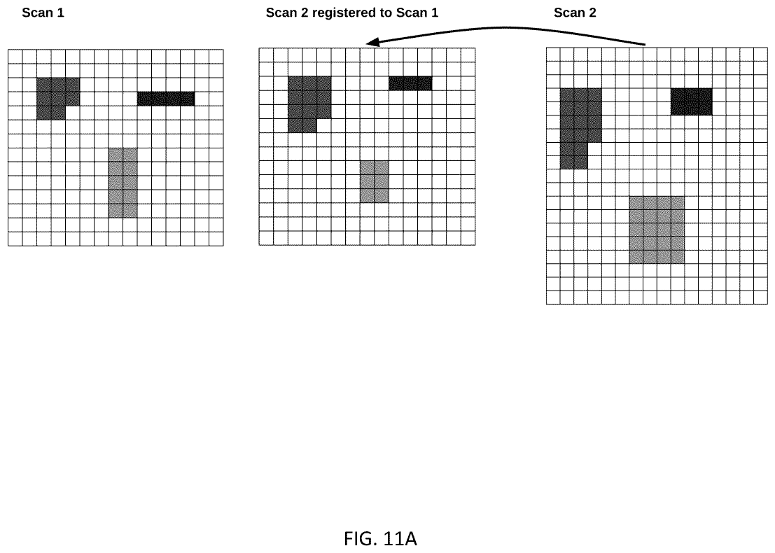

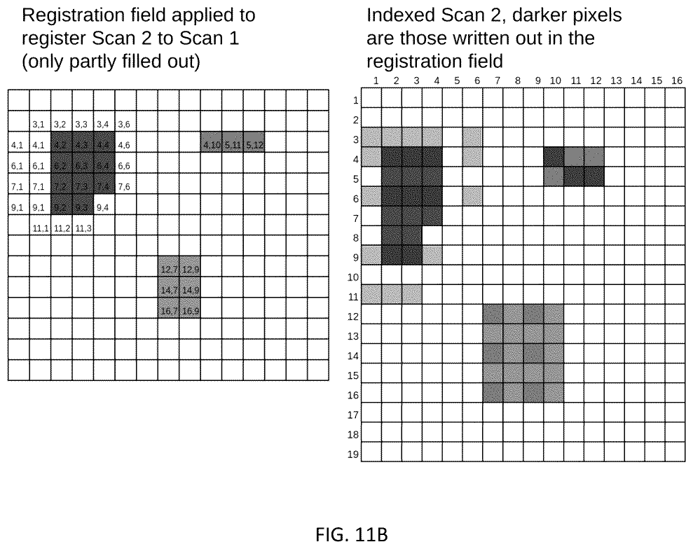

FIG. 11A shows a schematic of a second image registered to a first image, illustrating changes in voxels. FIG. 11B illustrates a schematic of a registration field, comprising vectors for a subset of the voxels. As illustrated in FIG. 11B, in certain embodiments, a registration field comprises references for positions (e.g., voxels) in a first image to a corresponding point (e.g., voxel) in a second image (target voxels in the second image are darkened in FIG. 11B). In certain embodiments, an inverted registration field may be determined. An inverted registration field comprises references for positions (e.g., voxels) in a second image to positions (e.g., voxels) in a first image. In certain embodiments, inverted reference fields are first created for each of affine registration. The inverted fields may then be weighted together in the same way as the affine registrations to create a full body inverted registration field.

In certain embodiments, without wishing to be bound to any particular theory, a first scan resides in one space (e.g., in world coordinates) and a second scan in another space. Through finding a registration that best aligns (e.g., via finding a local optima in an optimization problem) the organ segmentation from the second scan to the organ segmentation in the first scan a registration field from the first image space to the second image space is created. The registration field can then be applied to any image (e.g. PET, CT, organ segmentation, hotspot map) that resides in the same space as the second scan to register it to the space of the first scan.

Pointwise Registration

Additionally or alternatively, in certain embodiments, approaches described herein can be used to create a pointwise registration 850. In certain embodiments, a pointwise registration can be used to, e.g., triangulate between two PET/CT image stacks taken at two different time points. In certain embodiments, as described herein, a pointwise registration approach uses “anchor points”, which are single point correspondences, for example as opposed to corresponding masks that identify corresponding 3D tissue regions (e.g., pelvic bones) as described above.

In certain embodiments, a pointwise registration approach utilizes anatomical segmentation maps determined for two different images, e.g. PET/CT images taken at two different time points for a same patient, to identify sets of anchor points. For example, a set of anchor points may be or include the following points: a center-of-mass of all left side ribs, a center-of-mass of all right side ribs, a center-of-mass of left hip bone(s), a center of mass of right hip bone(s), and a center-of-mass of a thoracic vertebrae. For a particular medical image, e.g., acquired at a particular time point, an anatomical segmentation map may be used to determine coordinates for each anchor point in a particular set of anchor points. Anchor point coordinates may, accordingly, be determined for each of a plurality of medical images, for example in a time series of medical images.

In certain embodiments, pointwise registration approaches determine transformation operations, such as translations, that match corresponding anchor points between two images. For example, in certain embodiments, a set of anchor points may include N anchor points. Coordinate values (e.g., (x, y, z) coordinates, in three dimensions) may be computed for each of the N anchor points, in a first and second image, which are to be registered with each other. For each anchor point, i, in the set, an individual anchor point translation \vec{Fᵢ} that matches its location the first image with its location in the second image can be determined. The individual anchor point translations can then be used to determine, for a particular point in the first image, a weighted translation, \vec{F_{W}}, that aligns it with or identifies a corresponding point (e.g., that represents a same underlying physical location) in the second image.

For example, for a particular selected point and a set of N anchor points, the weighted translation \vec{F_{W}} can be determined based on a (inverse) distance weighted sum of the individual anchor point translations, with each individual anchor point translation weighted (e.g., multiplied) by the inverse of its distance from the particular selected point. This particular pointwise registration approach may be expressed, for example, according to equation (14), below:

$$\vec{{F}_{W}} = \frac{{Σ}_{i = 1}^{N} \frac{1}{{D}_{i}} \vec{{F}_{l}}}{{Σ}_{j = 1}^{N} \frac{1}{{D}_{j}}} \qquad ( 14 )$$

where Dᵢ is the distance from the particular selected point to the i^{th} anchor point, \vec{F_{ι}} is the translation that matches the coordinate values of the i^{th} anchor point in the two images. Accordingly, \vec{F_{W}} is the weighted translation calculated for the particular (chosen) point, based on all the distances to the anchor points.

Turning again to FIGS. 7 and 8, registration fields and/or pointwise registrations 850 determined as described herein may be used to transform second and/or first hotspot map(s), 824 and/or 814, respectively, to register them 708, 852 with each other. In this manner, sets of hotspots identified within the different (e.g., first and second) medical images may be brought into alignment, allowing corresponding hotspots that represent a same physical lesion to be accurately identified 710, 854.

In certain embodiments, additionally or alternatively, registration fields and/or pointwise registrations may be determined as described herein and used to register a second medical image with a first medical image (e.g., collected at an earlier time), for example before a second hotspot map is created. The registered version of the second medical image may be used to create the second hotspot map, which, by virtue of having been created from the registered version of the second medical image, will be registered with a first hotspot map created from the first medical image.

Identifying Corresponding Hotspots

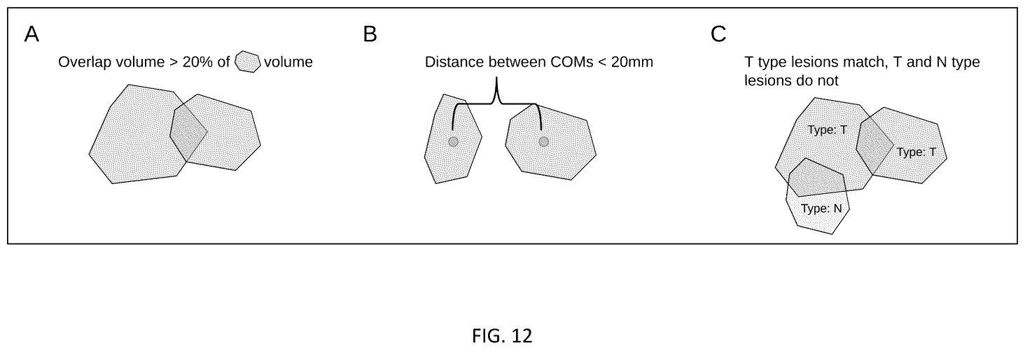

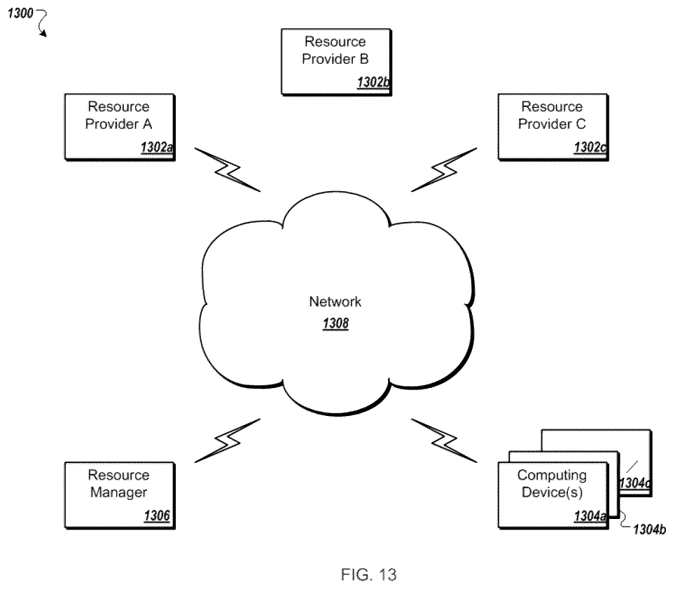

Turning to FIG. 12, in certain embodiment, corresponding hotspots may be identified by computing one or more lesion correspondence metrics that, for example, quantify a proximity and/or similarity between two or more hotspots identified in different medical images. Example metrics include, but are not limited to the following:

Hotspot overlap: In certain embodiments, hotspots in a first and second image that overlap (following registration) may be identified as corresponding hotspots for inclusion in a lesion correspondence. In certain embodiments, a relative fraction (percentage) of volume overlap may be computed and compared with one or more overlap threshold values. Hotspots having overlap fractions above a particular threshold value (e.g., 20 percent or more, 30 percent or more, 40 percent or more, 50 percent or more, 70 percent or more) may be identified as a lesion correspondence, e.g., as illustrated in panel A of FIG. 12.

Hotspot distance: In certain embodiments, for example, as shown in panel B of FIG. 12, a hotspot distance may be computed, for example as a distance between two points, such as a center of mass (COM) of each hotspot. Hotspot pairs separated by a hotspot distance of less than a particular distance threshold value, such as 10 mm or less, 20 mm or less, 30 mm or less, 40 mm or less, 50 mm or less, etc. may be identified as belonging to a lesion correspondence. In certain embodiments, multiple distance threshold values are used, for example for different regions. For example, in certain embodiments, a larger threshold (e.g., 50 mm) is used in a rib/chest region to account for respiratory motion and a smaller distance threshold (e.g., 10 mm, 20 mm, etc.) elsewhere.

Type/Location match: In certain embodiments, each hotspot may be assigned a lesion classification (e.g., an miTNM classification) and/or a location (e.g., pelvic, bone, lymph). In certain embodiments, hotspots may be required to have matching lesion classifications and/or assigned locations to be identified as corresponding hotspots in a lesion correspondence.

In this manner, hotspots appearing in different images can be matched with each other 854 and identified as representing to a same underlying physical lesion. Correspondences between such matching hotspots can be encoded via lesion correspondences that identifies corresponding hotspots in two or more different medical images (e.g., a first and a second image) can be created. Lesion correspondences may be bidirectional.

Lesion Tracking Metrics

In certain embodiments, systems and methods described herein provide for computation of metrics 712 that can be used to classify patient disease for treatment/decision making purposes and/or to stratify groups for clinical trial data collection and analysis 714. As described herein, such metrics may include a total lesion volume, e.g., computed as a sum of hotspot volumes over an entire subject, and/or a change thereof, as well as a number of newly identified lesions and/or lack thereof (or reduction in number of total lesions), as well as other metrics, for example various hotspot quantification and/or patient indices/metrics described herein, for example in Sections D and E. In certain embodiments, such metrics may be shown in a report, for examples in a tabular format or as a series of graphs or traces in a graph, for example as shown in FIG. 4B. In certain embodiments, values of normal (non-cancerous) physiological uptake may also be displayed, as shown in FIG. 4B.

In certain embodiments, approaches described herein for identifying corresponding hotspots may be used to match other target regions (e.g., corresponding to other physical features of the subject) identified within different images, e.g., collected at different times, from different subjects, with different tracers, etc. Such approaches may be employed to align and identify corresponding target regions identified within different images to assess presence, progression, state, response to treatment, etc. of a variety of conditions, not necessarily limited to cancer, such as muscle, ligament, tendon injuries, diagnosis of aneurysms, assessing cognitive activity (e.g., via fMRI), and the like.

G. Informing Clinical Decision Making and Treatment Evaluation

In certain embodiments, metrics computed based on analysis of images as described herein may, in turn, be used to determine values of, and/or stratify subjects according to various metrics indicative of disease state, progression, prognosis, subject response to therapy and/or a prediction of a likely subject response to one or more particular therapies etc.

In certain embodiments, these metrics may be themselves and/or be correlated with endpoints, such as clinical endpoints (e.g., which measure how a patient functions, feels, or survives) and may be used to evaluate treatment efficacy, for example in the context of population analyses in clinical trials and may be used alone and/or in combination with other markers, such as prostate specific antigen (PSA).

In certain embodiments, endpoints that may be determined and/or correlated with patient metrics and/or classifications described herein include, but are not limited to overall survival (OS), radiographic progression free survival (rPFS), various symptom endpoints (e.g., patient-reported outcomes), disease free survival (DFS), event-free survival (EFS), objective response rate (ORR), complete response (CR)/partial response (PR)/stable disease (SD)/progressive disease (PD), progression free survival (PFS), time to progression (TTP), a time to radiographic progression.

In certain embodiments, various metrics described herein and/or endpoint values determined therefrom may be used for guiding treatment decisions. For example approaches described herein may be used identify whether or not a subject is a responder to a particular treatment, providing the opportunity to discontinue ineffective treatment, adjust dosages, or switch to a new therapy, early on.

Accordingly, among other things, image analysis and decision support tools described herein may be used to determine prognostic information, measure response to therapy, stratify patients for radioligand therapy, and/or provide predictive information for other therapies.

For example, in certain embodiments, metrics computed from images as described herein, such as miTNM classifications of individual lesions and/or overall disease stage (as shown, e.g., in FIG. 4A), expression scores, a PRIMARY score, measures of tumor volume (e.g., total tumor volume for a patient and/or stratified by lesion class), presence and/or count of new lesions, may be used to compute specific response classifications. For example, lesion tracking tools described herein may be used to identify new lesions and quantify increase in tumor size, changes in an aPSMA score (e.g., a lesion index score and/or intensity-weighted total volume, as described herein) which, in turn, may be used to evaluate prostate cancer progression criteria, such as a PSMA PET Progression (PPP) score (see, e.g., Fanti et al., Proposal of Systemic Therapy Response Assessment Criteria in time of PSMA PET/CT imaging: PSMA PET Progression (PPP),” J. Nucl. Med., 2019 https://doi.org/10.2967/jnumed.119.233817), RECIP criteria score, and the like.

In certain embodiments, patient index quantification values at single and/or multiple time points may be used as inputs to prognostic models to determine prognostic metrics that are indicative of and/or quantify a likelihood of a particular clinical event, disease recurrence, or progression in patients (e.g., with or at risk for prostate cancer). Prognostic metrics may include overall survival (OS), radiographic progression free survival (rPFS), various symptom endpoints (e.g., patient-reported outcomes), disease free survival (DFS), event-free survival (EFS), objective response rate (ORR), complete response (CR)/partial response (PR)/stable disease (SD)/progressive disease (PD), progression free survival (PFS), time to progression (TTP), time to radiographic progression.

Prognostic models may be statistical models, such as regressions, and may include additional, clinical variable, inputs, such as patient physical characteristics such as a race/ethnicity; a prostate specific antigen (PSA) level and/or velocity; a hemoglobin level; a lactate dehydrogenase level; an albumin level; a clinical T stage; a biopsy Gleason score; and a percentage positive core score. In certain embodiments, prognostic models compare computed values, such as patient indices to one or more thresholds to classify patients and/or place them in a ‘bucket’ such as one of a set of ranges of OS values, etc. In certain embodiments prognostic models may be machine learning models—for example, various individual hotspot quantification metrics and/or aggregated patient-level indices may be taken as features—inputs—to a machine learning model that generates, as output, a predicted value for one or more of prognostic endpoints described herein. Such machine learning models may be, for example, artificial neural networks (ANNs). Machine learning models may also include clinical variables as inputs (i.e., features).

For example, in certain embodiments quantitative measure of disease burden from a single time point may be used to compute values of patient-level metrics such as a total tumor volume, an overall measure of intensity, such as a total SUV mean/max/peak, an aPSMA score (e.g., an intensity-weighted total volume). These metrics may be used as input to a prognostic model to generate, as output, one or more of: an expected survival (e.g., in months), a time to progression (TTP), and a time to radiographic progression.

In certain embodiments, quantitative data over multiple time points, such as change in total lesion volume, SUV, aPSMA score, measures of lesion changes over time (e.g., number of new lesions, number of disappeared lesions, number of tracked lesions) may be used as input to a prognostic model to generate, as output, one or more of: an expected survival (e.g., in months), a time to progression, a time to radiographic progression.

In certain embodiments, additionally or alternatively, characteristics of PSMA expression, e.g., in a prostate (and/or other tissue regions, e.g., which may be identified via anatomical segmentation techniques described herein) can be used as inputs to prognostic models. For example, spatial intensity patterns (e.g., from intensities of a functional image, such as a PET or SPECT image) in particular tissue regions may be used as input to a machine learning module, alone and/or with quantitative metrics and clinical variables described herein, to generate predictions, such as a risk of concurrent (synchronous) metastases, risk of future (metachronous) metastasis. For example, data from lesion tracking techniques described herein may be used as input to improve prediction techniques such as those described in U.S. Pat. No. 11,564,621, the content of which is hereby incorporated by reference in its entirety. In certain embodiments, intensity patterns may be used to determine, e.g., for each image of a subject at a particular time point, a score, such as or analogous to a PRIMARY score, as described in Siefert et al., “Second Version of the Prostate Cancer Molecular Imaging Standardized Evaluation Framework Including Response Evaluation for Clinical Trials (PROMISE V2),” Eur Urol. 2023 May; 83(5):405-412. doi: 10.1016/j.eururo.2023.02.002. Such automatically computed intensity scores may be included in patient reports, for example such as those shown in FIG. 4A.

In certain embodiments, approaches described herein may be used to generate models for categorizing patient response to therapy. For example, lesion tracking techniques may described herein may be used to determine inputs such as changes in tumor volume, intensity, appearance/disappearance of lesions. These inputs may be used via one or more response models to determine whether a patient is responding to treatment (e.g., a yes/no classification) and/or degree to which patient is responding to treatment (e.g., a numerical values). As described herein, such approaches may leverage existing response criteria, such as RECIP and PPP, which currently rely on variable and time consuming manual radiologist assessments and, accordingly, may be improved by the present techniques to improve accuracy, robustness (e.g., uniformity across different operators, imaging sites, etc.) and speed of patient staging and response to therapy evaluation.

In certain embodiments, approaches described herein may be used to evaluate which patients are likely to experience favorable benefit and/or unfavorable effects from particular treatments, which may e.g., be expensive and/or associate with adverse side effects. For example, software may be used to provide an indication of whether a patient is likely to benefit from a particular radioligand therapy. In this manner, approaches described herein may satisfy a significant unmet need in radioligand therapy (e.g., Pluvicto™) and help physician navigate between the large and growing number of therapies, especially in late-stage disease. For example, for a set of possible treatments (e.g., Abiraterone, Enzalutamide, Apalutamide, Darolutamide, Sipuleucel-T, Ra223, Docetaxel, Carbazitaxel, Pembrolizumab, Olaparib, Rucaparib, 177Lu-PSMA617, etc.), a predictive model may receive, as input, various imaging metrics described herein and generate, as output, a score for each treatment (or class of treatments, such as particular therapeutic classes—e.g., androgen biosynthesis inhibitors (e.g., Abiraterone), androgen receptor inhibitors (e.g., Enzalutamide, Apalutamide, Darolutamide), cellular immunotherapies (e.g., Sipuleucel-T), internal radiotherapy treatments (e.g., Ra223), antineoplastics (e.g., Docetaxel, Carbazitaxel), immune checkpoint inhibitors (Pembrolizumab), PARP inhibitors (e.g., Olaparib, Rucaparib), PSMA binding agents (e.g., with a radioligand therapy, e.g., Lu177) indicative of a likelihood that the patient will respond positively to the treatment.

H. Imaging Agents

As described herein, a variety of radionuclide labelled PSMA binding agents may be used as radiopharmaceutical imaging agents for nuclear medicine imaging to detect and evaluate prostate cancer. In certain embodiments, certain radionuclide labelled PSMA binding agents are appropriate for PET imaging, while others are suited for SPECT imaging.

i. PET Imaging Radionuclide Labelled PSMA Binding Agents

In certain embodiments, a radionuclide labelled PSMA binding agent is a radionuclide labelled PSMA binding agent appropriate for PET imaging.

In certain embodiments, a radionuclide labelled PSMA binding agent comprises [18F]DCFPyL (also referred to as PyL™; also referred to as DCFPyL-18F):

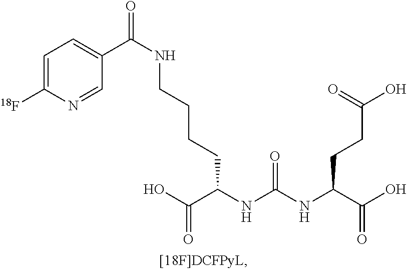 [SMILES: O=C(O)CC[C@H](NC(=O)N[C@@H](CCCCNC(=O)c1ccc([18F])nc1)C(=O)O)C(=O)O]

or a pharmaceutically acceptable salt thereof.

In certain embodiments, a radionuclide labelled PSMA binding agent comprises [18F]DCFBC:

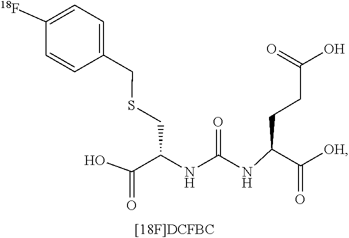 [SMILES: O=C(O)CC[C@H](NC(=O)N[C@@H](CSCc1ccc([18F])cc1)C(=O)O)C(=O)O]

or a pharmaceutically acceptable salt thereof.

In certain embodiments, a radionuclide labelled PSMA binding agent comprises ⁶⁸Ga-PSMA-HBED-CC (also referred to as ⁶⁸Ga-PSMA-11):

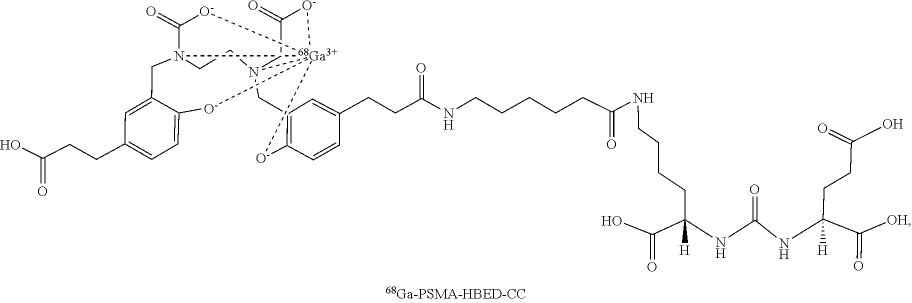

or a pharmaceutically acceptable salt thereof.

In certain embodiments, a radionuclide labelled PSMA binding agent comprises PSMA-617:

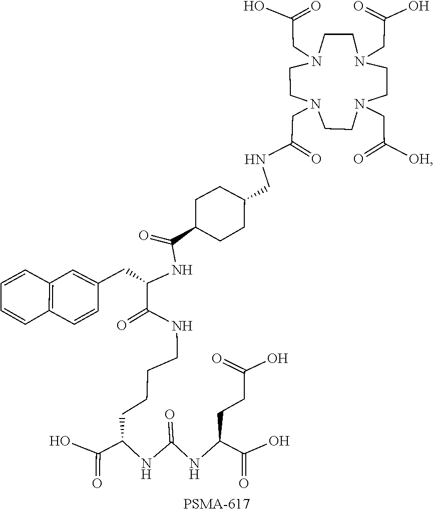

or a pharmaceutically acceptable salt thereof. In certain embodiments, the radionuclide labelled PSMA binding agent comprises ⁶⁸Ga-PSMA-617, which is PSMA-617 labelled with ⁶⁸Ga, or a pharmaceutically acceptable salt thereof. In certain embodiments, a radionuclide labelled PSMA binding agent comprises ¹⁷⁷Lu-PSMA-617, which is PSMA-617 labelled with ¹⁷⁷Lu, or a pharmaceutically acceptable salt thereof.

In certain embodiments, a radionuclide labelled PSMA binding agent comprises PSMA-I&T:

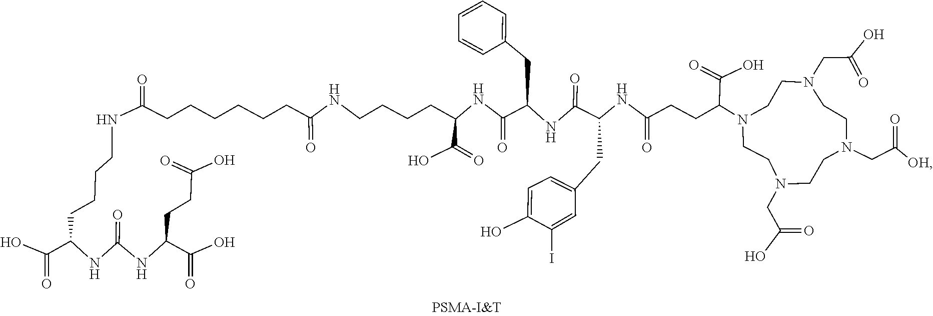 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

or a pharmaceutically acceptable salt thereof. In certain embodiments, a radionuclide labelled PSMA binding agent comprises ⁶⁸Ga-PSMA-I&T, which is PSMA-I&T labelled with ⁶⁸Ga, or a pharmaceutically acceptable salt thereof.

In certain embodiments, a radionuclide labelled PSMA binding agent comprises PSMA-1007:

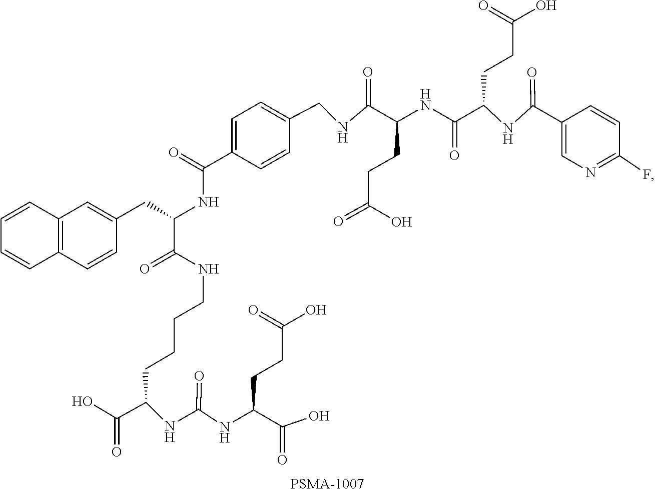

or a pharmaceutically acceptable salt thereof. In certain embodiments, a radionuclide labelled PSMA binding agent comprises ¹⁸F-PSMA-1007, which is PSMA-1007 labelled with ¹⁸F, or a pharmaceutically acceptable salt thereof.

In certain embodiments, a radionuclide labeled PSMA binding agent comprises 18F-JK-PSMA-7:

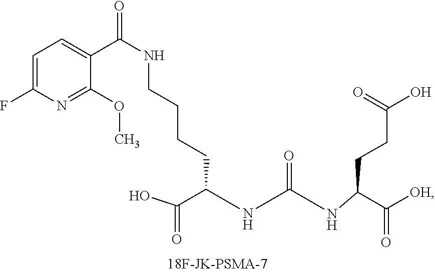 [SMILES: COc1nc(F)ccc1C(=O)NCCCC[C@H](NC(=O)N[C@@H](CCC(=O)O)C(=O)O)C(=O)O]

or a pharmaceutically acceptable salt thereof.

ii. SPECT Imaging Radionuclide Labelled PSMA Binding Agents

In certain embodiments, a radionuclide labelled PSMA binding agent is a radionuclide labelled PSMA binding agent appropriate for SPECT imaging.

In certain embodiments, a radionuclide labelled PSMA binding agent comprises 1404 (also referred to as MIP-1404):

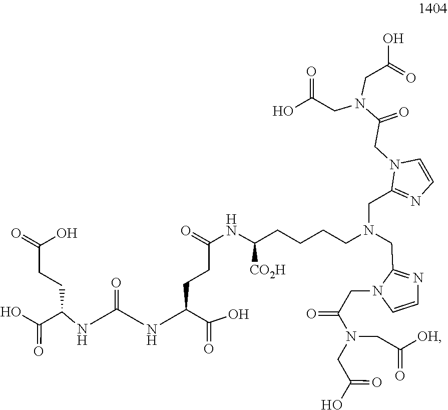 [SMILES: O=C(O)CC[C@H](NC(=O)N[C@@H](CCC(=O)N[C@@H](CCCCN(Cc1nccn1CC(=O)N(CC(=O)O)CC(=O)O)Cc1nccn1CC(=O)N(CC(=O)O)CC(=O)O)C(=O)O)C(=O)O)C(=O)O]

or a pharmaceutically acceptable salt thereof.

In certain embodiments, a radionuclide labelled PSMA binding agent comprises 1405 (also referred to as MIP-1405):

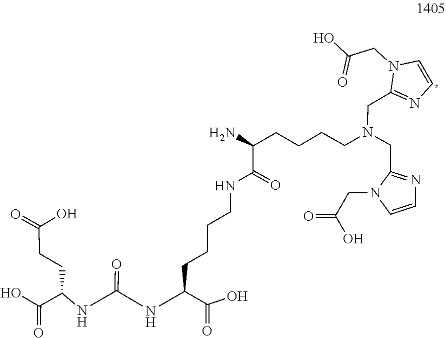 [SMILES: N[C@@H](CCCCN(Cc1nccn1CC(=O)O)Cc1nccn1CC(=O)O)C(=O)NCCCC[C@H](NC(=O)N[C@@H](CCC(=O)O)C(=O)O)C(=O)O]

or a pharmaceutically acceptable salt thereof.

In certain embodiments, a radionuclide labelled PSMA binding agent comprises 1427 (also referred to as MIP-1427):

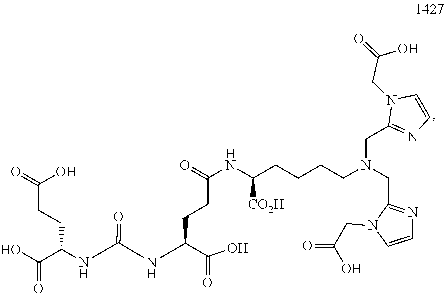 [SMILES: O=C(O)CC[C@H](NC(=O)N[C@@H](CCC(=O)N[C@@H](CCCCN(Cc1nccn1CC(=O)O)Cc1nccn1CC(=O)O)C(=O)O)C(=O)O)C(=O)O]

or a pharmaceutically acceptable salt thereof.

In certain embodiments, a radionuclide labelled PSMA binding agent comprises 1428 (also referred to as MIP-1428):

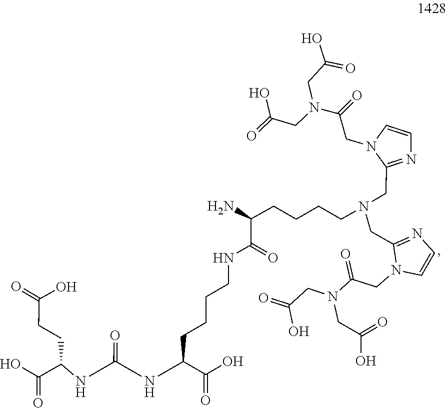 [SMILES: N[C@@H](CCCCN(Cc1nccn1CC(=O)N(CC(=O)O)CC(=O)O)Cc1nccn1CC(=O)N(CC(=O)O)CC(=O)O)C(=O)NCCCC[C@H](NC(=O)N[C@@H](CCC(=O)O)C(=O)O)C(=O)O]

or a pharmaceutically acceptable salt thereof.

In certain embodiments, a PSMA binding agent is labelled with a radionuclide by chelating it to a radioisotope of a metal [e.g., a radioisotope of technetium (Tc) (e.g., technetium-99m (^{99m}Tc)); e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)].

In certain embodiments, 1404 is labelled with a radionuclide (e.g., chelated to a radioisotope of a metal). In certain embodiments, a radionuclide labelled PSMA binding agent comprises ^{99m}Tc-MIP-1404, which is 1404 labelled with (e.g., chelated to)^{99m}Tc:

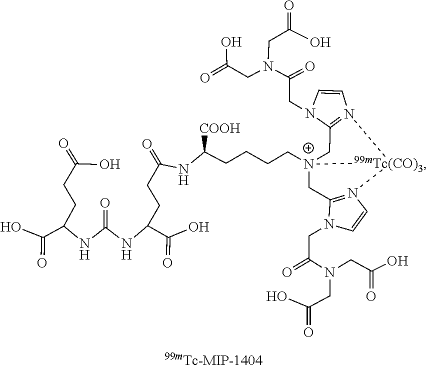

or a pharmaceutically acceptable salt thereof. In certain embodiments, 1404 may be chelated to other metal radioisotopes [e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] to form a compound having a structure similar to the structure shown above for ^{99m}Tc-MIP-1404, with the other metal radioisotope substituted for ^{99m}Tc.

In certain embodiments, 1405 is labelled with a radionuclide (e.g., chelated to a radioisotope of a metal). In certain embodiments, a radionuclide labelled PSMA binding agent comprises ^{99m}Tc-MIP-1405, which is 1405 labelled with (e.g., chelated to)^{99m}Tc:

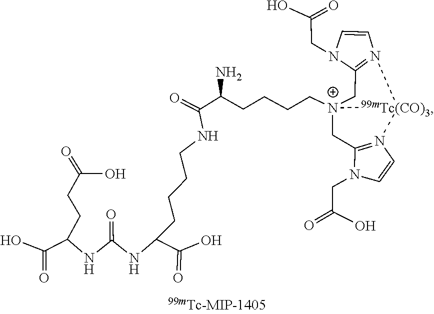

or a pharmaceutically acceptable salt thereof. In certain embodiments, 1405 may be chelated to other metal radioisotopes [e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] to form a compound having a structure similar to the structure shown above for ^{99m}Tc-MIP-1405, with the other metal radioisotope substituted for ^{99m}Tc.

In certain embodiments, 1427 is labelled with (e.g., chelated to) a radioisotope of a metal, to form a compound according to the formula below:

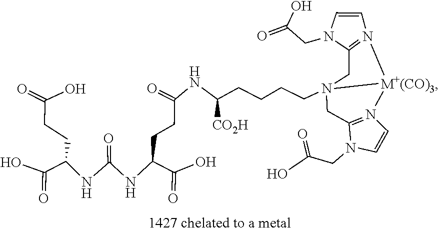

or a pharmaceutically acceptable salt thereof, wherein M is a metal radioisotope [e.g., a radioisotope of technetium (Tc) (e.g., technetium-99m (^{99m}Tc)); e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] with which 1427 is labelled.

In certain embodiments, 1428 is labelled with (e.g., chelated to) a radioisotope of a metal, to form a compound according to the formula below:

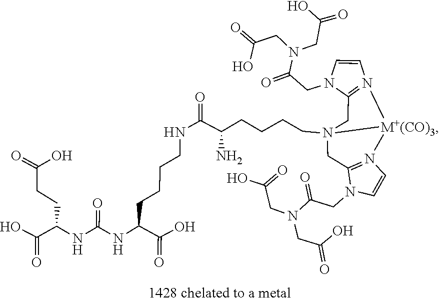

or a pharmaceutically acceptable salt thereof, wherein M is a metal radioisotope [e.g., a radioisotope of technetium (Tc) (e.g., technetium-99m (^{99m}Tc)); e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] with which 1428 is labelled.

In certain embodiments, a radionuclide labelled PSMA binding agent comprises PSMA I&S:

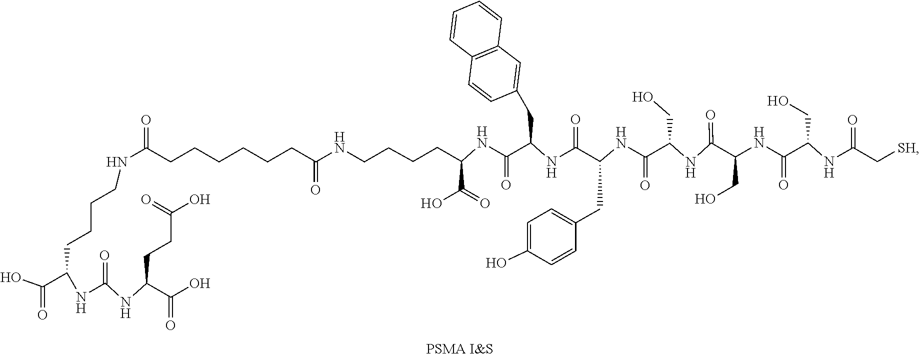 [SMILES: O=C(O)CC[C@H](NC(=O)N[C@@H](CCCCNC(=O)CCCCCCC(=O)NCCCC[C@@H](NC(=O)[C@@H](Cc1ccc2ccccc2c1)NC(=O)[C@@H](Cc1ccc(O)cc1)NC(=O)[C@H](CO)NC(=O)[C@H](CO)NC(=O)[C@H](CO)NC(=O)CS)C(=O)O)C(=O)O)C(=O)O]

or a pharmaceutically acceptable salt thereof. In certain embodiments, a radionuclide labelled PSMA binding agent comprises ^{99m}Tc-PSMA I&S, which is PSMA I&S labelled with ^{99m}Tc, or a pharmaceutically acceptable salt thereof.

I. Computer System and Network Environment

Certain embodiments described herein make use of computer algorithms in the form of software instructions executed by a computer processor. In certain embodiments, the software instructions include a machine learning module, also referred to herein as artificial intelligence software. As used herein, a machine learning module refers to a computer implemented process (e.g., a software function) that implements one or more specific machine learning techniques, e.g., artificial neural networks (ANNs), e.g., convolutional neural networks (CNNs), e.g., recursive neural networks, e.g., recurrent neural networks such as long short-term memory (LSTM) or Bilateral long short-term memory (Bi-LSTM), random forest, decision trees, support vector machines, and the like, in order to determine, for a given input, one or more output values.

In certain embodiments, machine learning modules implementing machine learning techniques are trained, for example using datasets that include categories of data described herein (e.g., CT images, MRI images, PET images, SPECT images). Such training may be used to determine various parameters of machine learning algorithms implemented by a machine learning module, such as weights associated with layers in neural networks. In certain embodiments, once a machine learning module is trained, e.g., to accomplish a specific task such as segmenting anatomical regions, segmenting and/or classifying hotspots, or determining values for prognostic, treatment response, and/or predictive metrics, values of determined parameters are fixed and the (e.g., unchanging, static) machine learning module is used to process new data (e.g., different from the training data) and accomplish its trained task without further updates to its parameters (e.g., the machine learning module does not receive feedback and/or updates). In certain embodiments, machine learning modules may receive feedback, e.g., based on user review of accuracy, and such feedback may be used as additional training data, to dynamically update the machine learning module. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of a ANN module may be carried out via specialized hardware (e.g., via an application specific integrated circuit (ASIC)).

As shown in FIG. 13, an implementation of a network environment 1300 for use in providing systems, methods, and architectures as described herein is shown and described. In brief overview, referring now to FIG. 13, a block diagram of an exemplary cloud computing environment 1300 is shown and described. The cloud computing environment 1300 may include one or more resource providers 1302a, 1302b, 1302c (collectively, 1302). Each resource provider 1302 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1302 may be connected to any other resource provider 1302 in the cloud computing environment 1300. In some implementations, the resource providers 1302 may be connected over a computer network 1308. Each resource provider 1302 may be connected to one or more computing device 1304a, 1304b, 1304c (collectively, 1304), over the computer network 1308.

The cloud computing environment 1300 may include a resource manager 1306. The resource manager 1306 may be connected to the resource providers 1302 and the computing devices 1304 over the computer network 1308. In some implementations, the resource manager 1306 may facilitate the provision of computing resources by one or more resource providers 1302 to one or more computing devices 1304. The resource manager 1306 may receive a request for a computing resource from a particular computing device 1304. The resource manager 1306 may identify one or more resource providers 1302 capable of providing the computing resource requested by the computing device 1304. The resource manager 1306 may select a resource provider 1302 to provide the computing resource. The resource manager 1306 may facilitate a connection between the resource provider 1302 and a particular computing device 1304. In some implementations, the resource manager 1306 may establish a connection between a particular resource provider 1302 and a particular computing device 1304. In some implementations, the resource manager 1306 may redirect a particular computing device 1304 to a particular resource provider 1302 with the requested computing resource.

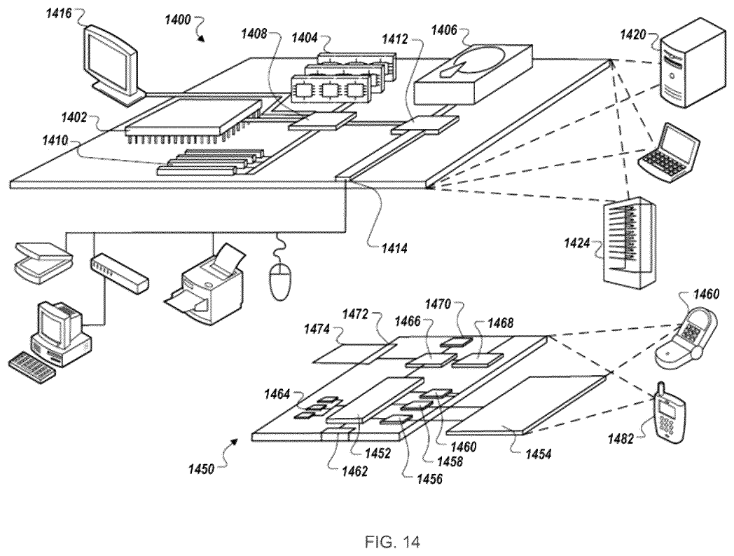

FIG. 14 shows an example of a computing device 1400 and a mobile computing device 1450 that can be used to implement the techniques described in this disclosure. The computing device 1400 is intended to represent various forms of digital computers, such as laptops, desktops, workstations, personal digital assistants, servers, blade servers, mainframes, and other appropriate computers. The mobile computing device 1450 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1400 includes a processor 1402, a memory 1404, a storage device 1406, a high-speed interface 1408 connecting to the memory 1404 and multiple high-speed expansion ports 1410, and a low-speed interface 1412 connecting to a low-speed expansion port 1414 and the storage device 1406. Each of the processor 1402, the memory 1404, the storage device 1406, the high-speed interface 1408, the high-speed expansion ports 1410, and the low-speed interface 1412, are interconnected using various busses, and may be mounted on a common motherboard or in other manners as appropriate. The processor 1402 can process instructions for execution within the computing device 1400, including instructions stored in the memory 1404 or on the storage device 1406 to display graphical information for a GUI on an external input/output device, such as a display 1416 coupled to the high-speed interface 1408. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 1404 stores information within the computing device 1400. In some implementations, the memory 1404 is a volatile memory unit or units. In some implementations, the memory 1404 is a non-volatile memory unit or units. The memory 1404 may also be another form of computer-readable medium, such as a magnetic or optical disk.

The storage device 1406 is capable of providing mass storage for the computing device 1400. In some implementations, the storage device 1406 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 1402), perform one or more methods, such as those described above. The instructions can also be stored by one or more storage devices such as computer- or machine-readable mediums (for example, the memory 1404, the storage device 1406, or memory on the processor 1402).

The high-speed interface 1408 manages bandwidth-intensive operations for the computing device 1400, while the low-speed interface 1412 manages lower bandwidth-intensive operations. Such allocation of functions is an example only. In some implementations, the high-speed interface 1408 is coupled to the memory 1404, the display 1416 (e.g., through a graphics processor or accelerator), and to the high-speed expansion ports 1410, which may accept various expansion cards (not shown). In the implementation, the low-speed interface 1412 is coupled to the storage device 1406 and the low-speed expansion port 1414. The low-speed expansion port 1414, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1400 may be implemented in a number of different forms, as shown in the figure. For example, it may be implemented as a standard server 1420, or multiple times in a group of such servers. In addition, it may be implemented in a personal computer such as a laptop computer 1422. It may also be implemented as part of a rack server system 1424. Alternatively, components from the computing device 1400 may be combined with other components in a mobile device (not shown), such as a mobile computing device 1450. Each of such devices may contain one or more of the computing device 1400 and the mobile computing device 1450, and an entire system may be made up of multiple computing devices communicating with each other.

The mobile computing device 1450 includes a processor 1452, a memory 1464, an input/output device such as a display 1454, a communication interface 1466, and a transceiver 1468, among other components. The mobile computing device 1450 may also be provided with a storage device, such as a micro-drive or other device, to provide additional storage. Each of the processor 1452, the memory 1464, the display 1454, the communication interface 1466, and the transceiver 1468, are interconnected using various buses, and several of the components may be mounted on a common motherboard or in other manners as appropriate.

The processor 1452 can execute instructions within the mobile computing device 1450, including instructions stored in the memory 1464. The processor 1452 may be implemented as a chipset of chips that include separate and multiple analog and digital processors. The processor 1452 may provide, for example, for coordination of the other components of the mobile computing device 1450, such as control of user interfaces, applications run by the mobile computing device 1450, and wireless communication by the mobile computing device 1450.

The processor 1452 may communicate with a user through a control interface 1458 and a display interface 1456 coupled to the display 1454. The display 1454 may be, for example, a TFT (Thin-Film-Transistor Liquid Crystal Display) display or an OLED (Organic Light Emitting Diode) display, or other appropriate display technology. The display interface 1456 may comprise appropriate circuitry for driving the display 1454 to present graphical and other information to a user. The control interface 1458 may receive commands from a user and convert them for submission to the processor 1452. In addition, an external interface 1462 may provide communication with the processor 1452, so as to enable near area communication of the mobile computing device 1450 with other devices. The external interface 1462 may provide, for example, for wired communication in some implementations, or for wireless communication in other implementations, and multiple interfaces may also be used.

The memory 1464 stores information within the mobile computing device 1450. The memory 1464 can be implemented as one or more of a computer-readable medium or media, a volatile memory unit or units, or a non-volatile memory unit or units. An expansion memory 1474 may also be provided and connected to the mobile computing device 1450 through an expansion interface 1472, which may include, for example, a SIMM (Single In Line Memory Module) card interface. The expansion memory 1474 may provide extra storage space for the mobile computing device 1450, or may also store applications or other information for the mobile computing device 1450. Specifically, the expansion memory 1474 may include instructions to carry out or supplement the processes described above, and may include secure information also. Thus, for example, the expansion memory 1474 may be provide as a security module for the mobile computing device 1450, and may be programmed with instructions that permit secure use of the mobile computing device 1450. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 1452), perform one or more methods, such as those described above. The instructions can also be stored by one or more storage devices, such as one or more computer- or machine-readable mediums (for example, the memory 1464, the expansion memory 1474, or memory on the processor 1452). In some implementations, the instructions can be received in a propagated signal, for example, over the transceiver 1468 or the external interface 1462.

The mobile computing device 1450 may communicate wirelessly through the communication interface 1466, which may include digital signal processing circuitry where necessary. The communication interface 1466 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1468 using a radio-frequency. In addition, short-range communication may occur, such as using a Bluetooth®, Wi-Fi™, or other such transceiver (not shown). In addition, a GPS (Global Positioning System) receiver module 1470 may provide additional navigation- and location-related wireless data to the mobile computing device 1450, which may be used as appropriate by applications running on the mobile computing device 1450.

The mobile computing device 1450 may also communicate audibly using an audio codec 1460, which may receive spoken information from a user and convert it to usable digital information. The audio codec 1460 may likewise generate audible sound for a user, such as through a speaker, e.g., in a handset of the mobile computing device 1450. Such sound may include sound from voice telephone calls, may include recorded sound (e.g., voice messages, music files, etc.) and may also include sound generated by applications operating on the mobile computing device 1450.

The mobile computing device 1450 may be implemented in a number of different forms, as shown in the figure. For example, it may be implemented as a cellular telephone 1480. It may also be implemented as part of a smart-phone 1482, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, various modules described herein can be separated, combined or incorporated into single or combined modules. Modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

1-34. (canceled)

35. A method for automated analysis of a time series of medical images of a subject, the method comprising:
(a) receiving and/or accessing, by a processor of a computing device, the time series of medical images of the subject; and
(b) identifying, by the processor, a plurality of hotspots within each of the medical images and determining, by the processor, one, two, or all three of (i), (ii), and (iii) as follows: (i) a change in the number of identified lesions (ii) a change in an overall volume of identified lesions, and (iii) a change in PSMA weighted total volume.

36. A method for analyzing a plurality of medical images of a subject, the method comprising:
(a) receiving and/or accessing, by a processor of a computing device, the plurality of medical images of the subject and obtaining, by the processor, a plurality of 3D hotspot maps, each corresponding to a particular medical image and identifying one or more hotspots within the particular medical image;
(b) for each particular one of the plurality of medical images, determining, by the processor, using a machine learning module, a corresponding 3D anatomical segmentation map that identifies a set of organ regions within the particular medical image, thereby generating a plurality of 3D anatomical segmentation maps;
(c) determining, by the processor, using (i) the plurality of 3D hotspot maps and (ii) the plurality of 3D anatomical segmentation maps, an identification of one or more lesion correspondences, each identifying two or more corresponding hotspots within different medical images and determined to represent a same underlying physical lesion within the subject; and
(d) determining, by the processor, based on the plurality of 3D hotspot maps and the identification of the one or more lesion correspondences, values of one or more metrics.

37. The method of claim 36, wherein the plurality of medical images comprise one or more anatomical images.

38. The method of claim 36, wherein the plurality of medical images comprise one or more nuclear medicine images.

39. The method of claim 36, wherein the plurality of medical images comprise one or more composite images, each comprising an anatomical and a nuclear medicine pair.

40. The method of claim 36, wherein the plurality of medical images are or comprises a time series of medical images, each medical image of the time series associated with and having been acquired at a different particular time.

41. The method of claim 40, where the time series of medical images comprises a first medical image acquired before administering a particular therapeutic agent to the subject and a second medical image acquired after administering the particular therapeutic agent to the subject.

42. The method of claim 41, comprising classifying the subject as a responder and/or a non-responder to the particular therapeutic agent based on the values of one or more metrics determined at step (d).

43. The method of claim 36, wherein step (a) comprises generating each hotspot map by segmenting at least a portion of the corresponding medical image.

44. The method of claim 36, wherein each hotspot map comprises, for each of at least a portion of the hotspots identified therein, one or more labels identifying one or more assigned anatomical regions and/or lesion sub-types.

45. The method of claim 36, wherein:
the plurality of hotspot maps comprises (i) a first hotspot map corresponding to a first medical image and (ii) a second hotspot map corresponding to a second medical image;
the plurality of 3D anatomical segmentation maps comprises (i) a first 3D anatomical segmentation map identifying the set of organ regions within the first medical image and (ii) a second 3D anatomical segmentation map identifying the set of organ regions within the second medical image; and
step (c) comprises registering (i) the first hotspot map with (ii) the second hotspot map using the first 3D anatomical segmentation map and the second 3D anatomical segmentation map.

46. The method of claim 36, wherein step (c) comprises:
determining, for a group of two or more hotspots, each a member of a different hotspot map and identified within a different medical image, values of one or more lesion correspondence metrics; and
determining the two or more hotspots of the group to represent a same particular underlying physical lesion based on the values of the one or more lesion correspondence metrics, thereby including the two or more hotspots of the group in one of the one or more lesion correspondences.

47. The method of claim 36, wherein step (d) comprises determining one, two, or all three of (i), (ii), and (iii) as follows: (i) a change in the number of identified lesions (ii) a change in an overall volume of identified lesions, and (iii) a change in PSMA.

48. The method of claim 36, comprising determining values of one or more prognostic metrics indicative of disease state/progression and/or treatment.

49. The method of claim 36, comprising using values of the one or more metrics as inputs to a prognostic model that generates, as output, an expectation value and/or range indicative of a likely value of a particular patient outcome.

50. The method of claim 36, comprising using values of the one or more metrics as inputs to a response model that generates, as output, a classification indicative of a patient response to treatment.

51. The method of claim 36, comprising using values of the one or more metrics as inputs to a predictive model that generates, as output, an eligibility score for each of one or more treatment options and/or classes of therapeutics, wherein the eligibility score for a particular treatment option and/or therapeutic class indicates a prediction of whether the patient will benefit from the particular treatment and/or therapeutic class.

52. A method for analyzing a plurality of medical images of a subject, the method comprising:
(a) obtaining, by a processor of a computing device, a first 3D hotspot map for the subject;
(b) obtaining, by the processor, a first 3D anatomical segmentation map associated with the first 3D hotspot map;
(c) obtaining, by the processor, a second 3D hotspot map for the subject;
(d) obtaining, by the processor, a second 3D anatomical segmentation map associated with the second 3D hotspot map;
(e) determining, by the processor, a registration field using/based on the first 3D anatomical segmentation map and the second 3D anatomical segmentation map;
(f) registering, by the processor, the first 3D hotspot map and the second 3D hotspot map, using the determined registration field, thereby generating a co-registered pair of 3D hotspot maps;
(g) determining, by the processor, an identification one or more lesion correspondences using the co-registered pair of 3D hotspot maps; and
(h) storing and/or providing, by the processor, the identification of the one or more lesion correspondences for display and/or further processing.

53. A method for analyzing a plurality of medical images of a subject, the method comprising:
(a) receiving and/or accessing, by a processor of a computing device, the plurality of medical images of the subject;
(b) for each particular one (medical image) of the plurality of medical images, determining, by the processor, using a machine learning module, a corresponding 3D anatomical segmentation map that identifies a set of organ regions within the particular medical image, thereby generating a plurality of 3D anatomical segmentation maps;
(c) determining, by the processor, using the plurality of 3D anatomical segmentation maps, one or more registration fields and applying the one or more registration fields to register the plurality of medical images, thereby creating a plurality of registered medical images;
(d) determining, by the processor, for each particular one of the plurality of registered medical images, a corresponding registered 3D hotspot map identifying one or more hotspots within the particular registered medical image, thereby creating a plurality of registered 3D hotspot maps;
(e) determining, by the processor, using the plurality of 3D registered hotspot maps, an identification of one or more lesion correspondences, each identifying two or more corresponding hotspots within different medical images and determined to represent a same underlying physical lesion within the subject; and
(f) determining, by the processor, based on the plurality of 3D hotspot maps and the identification of the one or more lesion correspondences, values of one or more metrics.

54. A method for analyzing a plurality of medical images of a subject, the method comprising:
(a) obtaining, by a processor of a computing device, a first 3D anatomical image and a first 3D functional image of the subject;
(b) obtaining, by the processor, a second 3D anatomical image and a second 3D functional image of the subject;
(c) obtaining, by the processor, a first 3D anatomical segmentation map based on the first 3D anatomical image;
(d) obtaining, by the processor, a second 3D anatomical segmentation map based on the second 3D anatomical image;
(e) determining, by the processor, a registration field using/based on the first 3D anatomical segmentation map and the second 3D anatomical segmentation map;
(f) registering, by the processor, the second 3D functional image to first 3D functional image using the registration field, thereby generating a registered version of the second 3D functional image;
(g) obtaining, by the processor a first 3D hotspot map associated with the first functional image;
(h) determining, by the processor, a second 3D hotspot map using the registered version of the second 3D functional image, the second 3D hotspot map thereby being registered with the first 3D hotspot map;
(i) determining, by the processor, an identification one or more lesion correspondences using the first 3D hotspot map and the second 3D hotspot map registered thereto; and
(j) storing and/or providing, by the processor, the identification of the one or more lesion correspondences for display and/or further processing.

55-58. (canceled)

59. A method of automated or semi-automated whole-body evaluation of a subject with metastatic prostate cancer to assess disease progression and/or treatment efficacy, the method comprising:
(a) receiving, by a processor of a computing device, a first prostate-specific membrane antigen (PSMA) targeting positron emission tomography (PET) image (the first PSMA-PET image) of the subject and a first 3D anatomical image of the subject, wherein the first 3D anatomical image of the subject is obtained simultaneously with or immediately subsequent to or immediately prior to the first PSMA PET image such that the first 3D anatomical image and the first PSMA PET image correspond to a first date, and wherein the images depict a large enough area of the subject's body to cover regions of the body to which the metastatic prostate cancer has spread;
(b) receiving, by the processor, a second PSMA-PET image of the subject and a second 3D anatomical image of the subject, both obtained on a second date subsequent to the first date;
(c) automatically determining, by the processor, a registration field using landmarks automatically identified within the first and second 3D anatomical images, and using, by the processor, the determined registration field to align the first and second PSMA-PET images; and
(d) using the thusly aligned first and second PSMA-PET images to automatically detect, by the processor, a change in the disease from the first date to the second date.

60. The method of claim 59, wherein the method comprises one or more members selected from the group consisting of lesion location assignment, tumor staging, nodal staging, distant metastasis staging, assessment of intraprostatic lesions, and determination of PSMA-expression score.

61. The method of claim 59, wherein the subject has administered to them a therapy for treatment of the metastatic prostate cancer at one or more times from the first date to the second date, such that the method is used to assess treatment efficacy.

62. The method of claim 59, further comprising obtaining a one or more further PSMA PET images and 3D anatomical images of the subject subsequent to the second date, aligning the further PSMA PET image(s) using corresponding 3D anatomical image(s), and using the aligned further PSMA PET image(s) to assess the disease progression and/or treatment efficacy.

63. The method of claim 59, further comprising determining and rendering, by the processor, a predicted PSMA-PET image depicting a predicted progression (or remission) of disease to a future date based at least in part on the detected change in the disease from the first date to the second date.

64-103. (canceled)
